(19) United States
(12) Patent Application Publication
(10) Pub. No.: US 2007/0173527 A1
(43) Pub. Date: Jul. 26, 2007

(54) HISTONE DEACETYLASE INHIBITORS

(76) Inventors: Jerome C. Bressi, San Diego, CA (US); Jason W. Brown, San Diego, CA (US); Anthony R. Gangloff, San Diego, CA (US); Andrew J. Jennings, San Diego, CA (US); Stephen W. Kaldor, San Diego, CA (US); Robert J. Skene, San Marcos, CA (US); Jeffrey A. Stafford, San Diego, CA (US); Phong H. Vu, San Diego, CA (US)

Correspondence Address: TAKEDA SAN DIEGO, INC., 10410 SCIENCE CENTER DRIVE, SAN DIEGO, CA, 92121, US

(21) Appl. No.: 11/622,669

(22) Filed: Jan. 12, 2007

Related U.S. Application Data
(60) Provisional application No. 60/758,935, filed on Jan. 13, 2006.

(51) Int. Cl. A61K 31/4745; A61K 31/454; A61K 31/4184; C07D 403/02; C07D 471/02

(52) U.S. Cl. 514/303; 514/384; 514/322; 546/118; 546/199; 548/307.1

(57) ABSTRACT

Compounds, pharmaceutical compositions, kits and methods are provided for use with histone deacetylases (HDACs) that comprise a compound selected from the group consisting of: 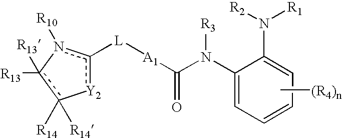 wherein the variables are as defined herein.

### RELATED APPLICATION

This application claims the benefit of U.S. Provisional Application No. 60/758,935, filed Jan. 13, 2006, which is incorporated herein by reference.

### FIELD OF THE INVENTION

The present invention relates to compounds that may be used to inhibit histone deacetylases (HDACs), as well as compositions of matter and kits comprising these compounds. The invention also relates to methods for inhibiting HDACs and treatment methods using compounds according to the present invention. In particular, the present invention relates to compounds, compositions of matter, kits and methods used to inhibit Class I HDACs, such as HDAC1, HDAC2, HDAC6 and HDAC8.

### BACKGROUND OF THE INVENTION

DNA in eukaryotic cells is tightly complexed with proteins (histones) to form chromatin. Histones are small, positively charged proteins that are rich in basic amino acids (positively charged at physiological pH), which contact the phosphate groups (negatively charged at physiological pH) of DNA. There are five main classes of histones Hi, H2A, H2B, H3, and H4. The amino acid sequences of H2A, H2B, H3, and H4 show remarkable conservation between species, wherein Hi varies somewhat and in some cases is replaced by another histone, e.g., H5. Four pairs of each of H2A, H2B, H3 and H4 together form a disk-shaped octomeric protein core, around which DNA (about 140 base pairs) is wound to form a nucleosome. Individual nucleosomes are connected by short stretches of linker DNA associated with another histone molecule to form a structure resembling a beaded string, which is itself arranged in a helical stack, known as a solenoid.

The majority of histones are synthesized during the S phase of the cell cycle, and newly synthesized histones quickly enter the nucleus to become associated with DNA. Within minutes of its synthesis, new DNA becomes associated with histones in nucleosomal structures.

A small fraction of histones, more specifically, the amino acid side chains thereof, are enzymatically modified by post-translational addition of methyl, acetyl, or phosphate groups, neutralizing the positive charge of the side chain, or converting it to a negative charge. For example, lysine and arginine groups may be methylated, lysine groups may be acetylated, and serine groups may be phosphorylated. For lysine, the —(CH₂)₄—NH₂ sidechain may be acetylated, for example by an acetyltransferase enzyme to give the amide —(CH₂)₄—NHC(═O)CH₃. Methylation, acetylation, and phosphorylation of amino termini of histones that extend from the nucleosomal core affect chromatin structure and gene expression. Spencer and Davie 1999. Gene 240:11-12.

Acetylation and deacetylation of histones is associated with transcriptional events leading to cell proliferation and/or differentiation. Regulation of the function of transcriptional factors is also mediated through acetylation. Recent reviews on histone deacetylation include Kouzarides et al., 1999, Curr. Opin. Genet. Dev. 9:1, 40-48 and Pazin et al., 1997, 89:3 325-328.

The correlation between acetylation status of histones and the transcription of genes has been known for quite some time. Certain enzymes, specifically acetylases (e.g., histone acetyltransferases (HAT) and deacetylases (histone deacetylases or HDACs), which regulate the acetylation state of histones have been identified in many organisms and have been implicated in the regulation of numerous genes, confirming a link between acetylation and transcription. In general, histone acetylation is believed to correlate with transcriptional activation, whereas histone deacetylation is believed to be associated with gene repression.

A growing number of histone deacetylases (HDACs) have been identified. HDACs function as part of large multiprotein complexes, which are tethered to the promoter and repress transcription. Well characterized transcriptional repressors such as MAD, nuclear receptors and YY1 associate with HDAC complexes to exert their repressor function.

Studies of HDAC inhibitors have shown that these enzymes play an important role in cell proliferation and differentiation. HDACs are believed to be associated with a variety of different disease states including, but not limited to cell proliferative diseases and conditions (Marks, P. A., Richon, V. M., Breslow, R. and Rifkind, R. A., J. Natl. Cancer Inst. (Bethesda) 92, 1210-1215, 2000) such as leukemia (Lin et al., 1998. Nature 391: 811-814; Grignani et al. 1998. Nature 391: 815-818; Warrell et al., 1998, J. Natl. Cancer Inst. 90:1621-1625; Gelmetti et al., 1998, Mol. Cell Biol. 18:7185-7191; Wang et al., 1998, PNAS 951 0860-10865), melanomas/squamous cell carcinomas (Gillenwater et al., 1998, Int. J. Cancer 75217-224; Saunders et al., 1999, Cancer Res. 59:399-404), breast cancer, prostrate cancer, bladder cancer (Gelmetti et al., 1998, Mol. Cell Biol. 18:7185-7191; Wang et al., 1998, PNAS 951 0860-10865), lung cancer, ovarian cancer and colon cancer (Hassig et al., 1997, Chem. Biol. 4:783-789; Archer et al., 1998, PNAS, 956791-6796; Swendeman et al., 1999, Proc. Amer. Assoc. Cancer Res. 40, Abstract #3836).

Histone deacetylase inhibitors are potent inducers of growth arrest, differentiation, or apoptotic cell death in a variety of transformed cells in culture and in tumor bearing animals (Histone deacetylase inhibitors as new cancer drugs, Marks, P. A., Richon, V. M., Breslow, R. and Rifkind, R. A., Current Opinions in Oncology, 2001, Nov. 13 (6): 477-83; Histone deacetylases and cancer: causes and therapies, Marks, P., Rifkind, R. A., Richon, V. M., Breslow, R., Miller, T. and Kelly, W. K., Nat. Rev. Cancer 2001 Dec. 1 (3):194-202). In addition, HDAC inhibitors are useful in the treatment or prevention of protozoal diseases (U.S. Pat. No. 5,922,837) and psoriasis (PCT Publication No. WO 02/26696).

Accordingly, despite the various HDAC inhibitors that have been reported to date, a need continues to exist for new and more effective inhibitors of HDACs.

### SUMMARY OF THE INVENTION

The present invention relates to compounds that have activity for inhibiting histone deacetylases (HDACs). The present invention also relates to pharmaceutical compositions, articles of manufacture and kits comprising these compounds. The invention further relates to method of using, methods for preparing and reagents useful in preparing the compounds according to the inventions.

In one aspect, the invention is directed to compounds having a specific formula.

In another aspect, the invention is directed to pharmaceutical compositions that comprises an HDAC inhibitor according to the present invention as an active ingredient. Pharmaceutical compositions according to the invention may optionally comprise 0.001%-100% of one or more HDAC inhibitors of this invention. These pharmaceutical compositions may be administered or coadministered by a wide variety of routes, including for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compositions may also be administered or coadministered in slow release dosage forms.

In another aspect, the invention is directed to kits and other articles of manufacture for treating disease states associated with HDAC. In one embodiment, a kit is provided that comprises a composition comprising at least one HDAC inhibitor of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another aspect, the invention is directed to articles of manufacture for treating disease states associated with HDAC. In one embodiment, an article of manufacture is provided that comprises a composition comprising at least one HDAC inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

In another aspect, the invention is directed to methods for preparing compounds, compositions and kits according to the present invention. In some embodiments, synthetic schemes are provided for synthesizing compounds according to the present invention.

In another aspect, the invention is directed to methods for using compounds, compositions, kits and articles of manufacture according to the present invention.

In one embodiment, the compounds, compositions, kits and articles of manufacture are used to inhibit one or more HDAC.

In another embodiment, the compounds, compositions, kits and articles of manufacture are used to treat a disease state for which one or more HDAC possesses activity that contributes to the pathology and/or symptomology of the disease state.

In another embodiment, a compound is administered to a subject wherein HDAC activity within the subject is altered, preferably reduced.

In another embodiment, a prodrug of a compound is administered to a subject that is converted to the compound in vivo where it inhibits one or more HDAC.

In another embodiment, a method of inhibiting one or more HDAC is provided that comprises contacting an HDAC with a compound according to the present invention.

In another embodiment, a method of inhibiting one or more HDAC is provided that comprises causing a compound according to the present invention to be present in a subject in order to inhibit the HDAC in vivo.

In another embodiment, a method of inhibiting an HDAC is provided that comprises administering a first compound to a subject that is converted in vivo to a second compound wherein the second compound inhibits the HDAC in vivo. It is noted that the compounds of the present invention may be the first or second compounds.

In another embodiment, a therapeutic method is provided that comprises administering a compound according to the present invention.

In another embodiment, a method of treating a condition in a patient which is known to be mediated by one or more HDAC, or which is known to be treated by HDAC inhibitors, comprising administering to the patient a therapeutically effective amount of a compound according to the present invention.

In another embodiment, a method is provided for treating a disease state for which one or more HDAC possesses activity that contributes to the pathology and/or symptomology of the disease state, the method comprising: causing a compound according to the present invention to be present in a subject in a therapeutically effective amount for the disease state.

In another embodiment, a method is provided for treating a disease state for which one or more HDAC possesses activity that contributes to the pathology and/or symptomology of the disease state, the method comprising: administering a first compound to a subject that is converted in vivo to a second compound such that the second compound is present in the subject in a therapeutically effective amount for the disease state. It is noted that the compounds of the present invention may be the first or second compounds.

In another embodiment, a method is provided for treating a disease state for which one or more HDAC possesses activity that contributes to the pathology and/or symptomology of the disease state, the method comprising: administering a compound according to the present invention to a subject such that the compound is present in the subject in a therapeutically effective amount for the disease state.

In another embodiment, a method is provided for using a compound according to the present invention in order to manufacture a medicament for use in the treatment of a disease state that is known to be mediated by one or more HDAC, or that is known to be treated by HDAC inhibitors.

It is noted in regard to all of the above embodiments that the present invention is intended to encompass all pharmaceutically acceptable ionized forms (e.g., salts) and solvates (e.g., hydrates) of the compounds, regardless of whether such ionized forms and solvates are specified since it is well know in the art to administer pharmaceutical agents in an ionized or solvated form. It is also noted that unless a particular stereochemistry is specified, recitation of a compound is intended to encompass all possible stereoisomers (e.g., enantiomers or diastereomers depending on the number of chiral centers), independent of whether the compound is present as an individual isomer or a mixture of isomers. Further, unless otherwise specified, recitation of a compound is intended to encompass all possible resonance forms and tautomers. With regard to the claims, the language “compound comprising the formula” is intended to encompass the compound and all pharmaceutically acceptable ionized forms and solvates, all possible stereoisomers, and all possible resonance forms and tautomers unless otherwise specifically specified in the particular claim.

It is further noted that prodrugs may also be administered which are altered in vivo and become a compound according to the present invention. The various methods of using the compounds of the present invention are intended, regardless of whether prodrug delivery is specified, to encompass the administration of a prodrug that is converted in vivo to a compound according to the present invention. It is also noted that certain compounds of the present invention may be altered in vivo prior to inhibiting kinases and thus may themselves be prodrugs for another compound. Such prodrugs of another compound may or may not themselves independently have kinase inhibitory activity.

### BRIEF DESCRIPTION OF THE FIGURES

FIG. 1 illustrates residues 1-482 of HDAC 1, plus a Flag tag at both the N- and C-terminus, and a Met residue cloning artifact at the N-terminus (SEQ ID NO: 1).

FIG. 2 illustrates the DNA sequence (SEQ ID NO: 2) that was used to encode SEQ ID NO: 1.

FIG. 3 illustrates residues 1-488 of HDAC2, plus a 6-histidine tag at the C-terminus and four residues of cloning artifact (a Met-Gly-Ser fragment at the N-terminus and a Gly in front of the 6-histidine tag) (SEQ ID NO: 3).

FIG. 4 illustrates the DNA sequence (SEQ ID NO: 4) that was used to encode SEQ ID NO: 3.

FIG. 5 illustrates residues 73-845 of HDAC6, plus a 6-histidine tag at the C-terminus and three residues of cloning artifact (Met-Pro pair at the N-terminus and a Gly in front of the 6-histidine tag) (SEQ ID NO: 5).

FIG. 6 illustrates the DNA sequence (SEQ ID NO: 6) that was used to encode SEQ ID NO: 5.

FIG. 7 illustrates residues 1-377 of HDAC8, plus a 6-histidine tag at the N-terminus and two residues of cloning artifact (a Met before and a Pro behind the 6-histidine tag) (SEQ ID NO: 7).

FIG. 8 illustrates the DNA sequence (SEQ ID NO: 8) that was used to encode SEQ ID NO: 7.

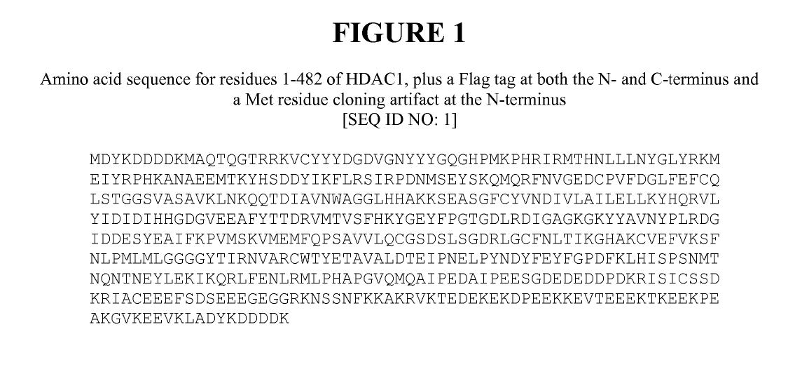

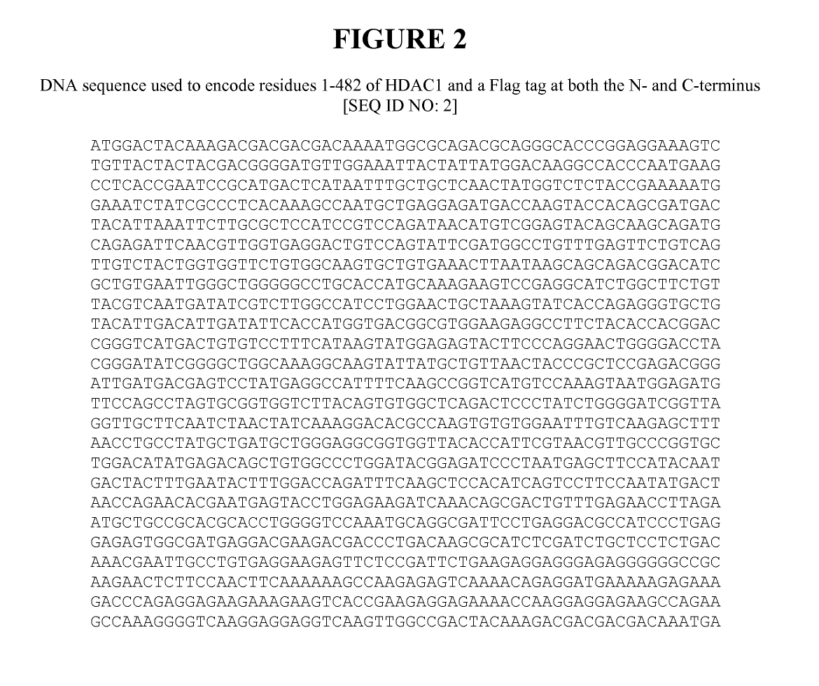

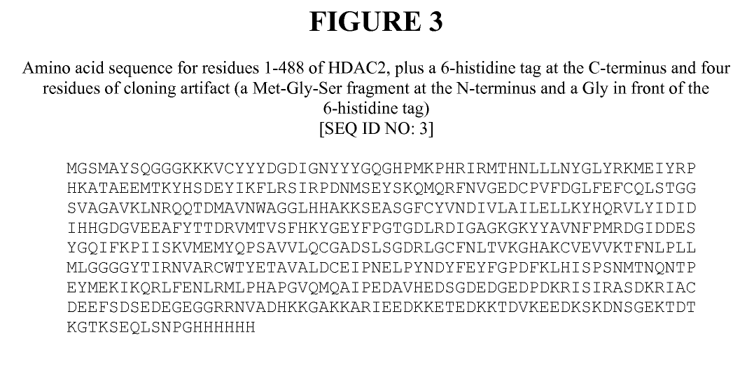

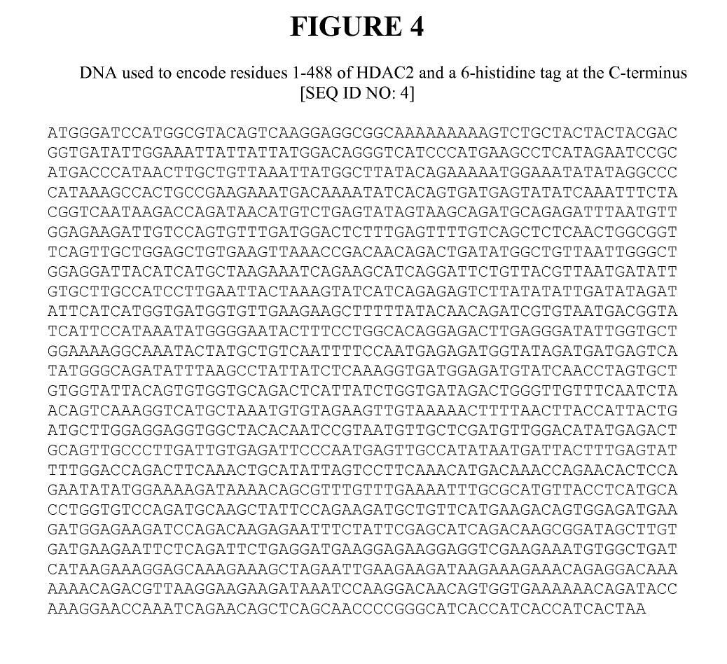

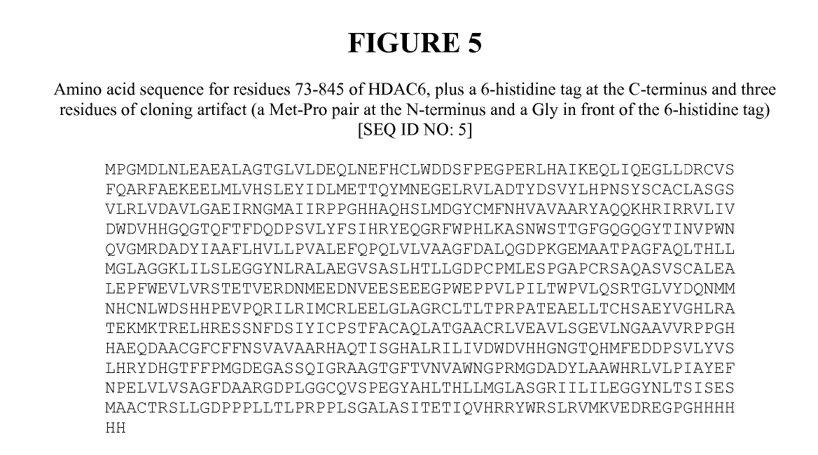

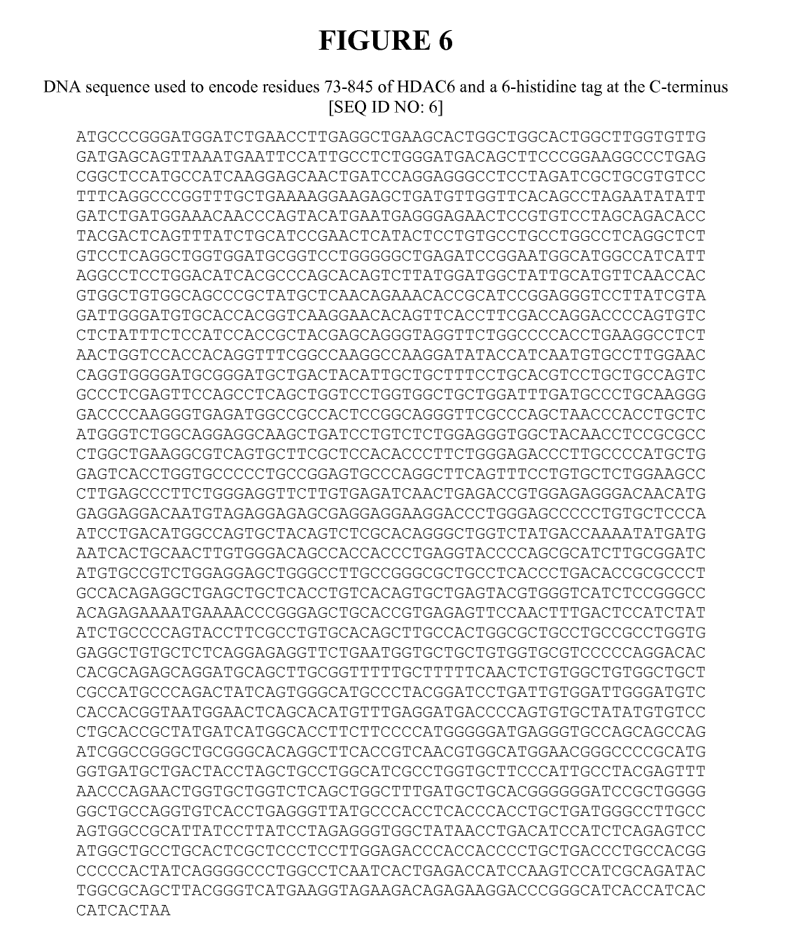

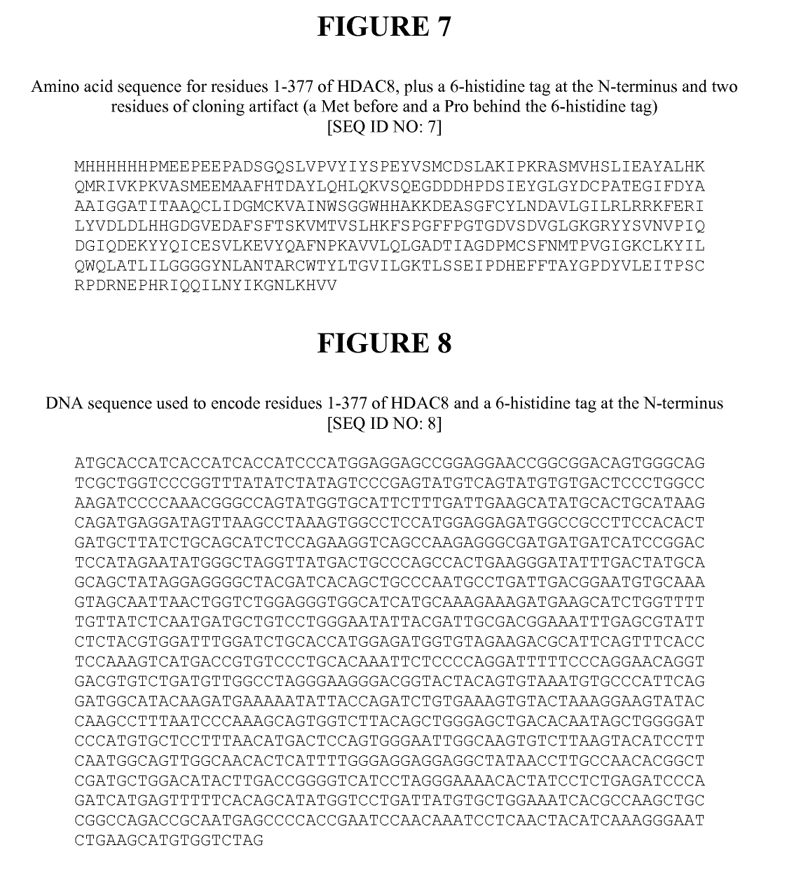

### DEFINITIONS

Unless otherwise stated, the following terms used in the specification and claims shall have the following meanings for the purposes of this Application.

“Alicyclic” means a moiety comprising a non-aromatic ring structure. Alicyclic moieties may be saturated or partially unsaturated with one, two or more double or triple bonds. Alicyclic moieties may also optionally comprise heteroatoms such as nitrogen, oxygen and sulfur. The nitrogen atoms can be optionally quaternerized or oxidized and the sulfur atoms can be optionally oxidized. Examples of alicyclic moieties include, but are not limited to moieties with C₃₋₈ rings such as cyclopropane, cyclohexane, cyclopentane, cyclopentene, cyclopentadiene, cyclohexane, cyclohexene, a moiety characterized by a straight or branched chain arrangement of constituent carbon cyclohexadiene, cycloheptane, cycloheptene, cycloheptadiene, cyclooctane, cyclooctene, and cyclooctadiene.

“Aliphatic” means atoms and may be saturated or partially unsaturated with one, two or more double or triple bonds.

“Alkoxy” means the radical —O-alkyl; the alkyl group is as defined in this and can be optionally substituted.

“Alkyl” represented by itself means a straight or branched, saturated or unsaturated, aliphatic radical having a chain of carbon atoms, optionally with oxygen (See “oxaalkyl”) or nitrogen atoms (See “azaalkyl”) between the carbon atoms. C_{X} alkyl and C_{X-Y} alkyl are typically used where X and Y indicate the number of carbon atoms in the chain. For example, C₁₋₆ alkyl includes alkyls that have a chain of between 1 and 6 carbons (e.g., methyl, ethyl, propyl, isopropyl, butyl, sec-butyl, isobutyl, tert-butyl, pentyl, hexyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl, heteroarylalkyl) means a straight or branched, saturated or unsaturated aliphatic divalent radical having the number of atoms indicated or when no atoms are indicated means a bond (e.g., (C₆₋₁₀)aryl(C₁₋₃)alkyl includes, benzyl, phenylethyl, 1-phenylethyl, 3-phenylpropyl, 2-thienylmethyl, 2-pyridinylmethyl and the like).

“Alkenyl” means a straight or branched, carbon chain that contains at least one carbon-carbon double bond. Examples of alkenyl include vinyl, allyl, isopropenyl, pentenyl, hexenyl, heptenyl, 1-propenyl, 2-butenyl, 2-methyl-2-butenyl, and the like.

“Alkynyl” means a straight or branched, carbon chain that contains at least one carbon-carbon triple bond. Examples of alkynyl include ethynyl, propargyl, 3-methyl-1-pentynyl, 2-heptynyl and the like.

“Alkylene”, unless indicated otherwise, means a straight or branched, saturated or unsaturated, aliphatic, divalent radical. C_{X} alkylene and C_{X-Y} alkylene are typically used where X and Y indicate the number of carbon atoms in the chain. For example, C₁₋₆ alkylene includes methylene (—CH₂—), ethylene (—CH₂CH₂—), trimethylene (—CH₂CH₂CH₂—), tetramethylene (—CH₂CH₂CH₂CH₂—), 2-butenylene (—CH₂CH═CHCH₂—), 2-methyltetramethylene (—CH₂CH(CH₃)CH₂CH₂—), pentamethylene (—CH₂CH₂CH₂CH₂CH₂—) and the like.

“Alkenylene” means a straight or branched, divalent carbon chain having one or more carbon-carbon double bonds. Examples of alkenylene include ethene-1,2-diyl, propene-1,3-diyl, methylene-1,1-diyl, and the like.

“Alkynylene” means a straight or branched, divalent carbon chain having one or more carbon-carbon triple bonds. Examples of alkynylene include ethyne-1,2-diyl, propyne-1,3-diyl, and the like.

“Alkylidene” means a straight or branched saturated or unsaturated, aliphatic radical connected to the parent molecule by a double bond. C_{X} alkylidene and C_{X-Y} alkylidene are typically used where X and Y indicate the number of carbon atoms in the chain. For example, C₁₋₆ alkylidene includes methylene (═CH₂), ethylidene (═CHCH₃), isopropylidene (═C(CH₃)₂), propylidene (═CHCH₂CH₃), allylidene (═CH—CH═CH₂), and the like).

“Amino” means the radical —NRₐR_{b}, where Rₐ and R_{b} are each independently hydrogen or a non-hydrogen substituent. Representative amino groups include, without limits, —NH₂, —NHCH₃, —N(CH₃)₂, —NHC₁₋₁₀-alkyl, —N(C₁₋₁₀-alkyl)₂, —NHaryl, —NHheteroaryl, —N(aryl)₂, —N(heteroaryl)₂, and the like. Optionally, Rₐ and R_{b} together with the nitrogen may also form a ring. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Animal” includes humans, non-human mammals (e.g., dogs, cats, rabbits, cattle, horses, sheep, goats, swine, deer, and the like) and non-mammals (e.g., birds, and the like).

“Aromatic” means a moiety wherein the constituent atoms make up an unsaturated ring system, all atoms in the ring system are Sp² hybridized and the total number of pi electrons is equal to 4n+2. An aromatic ring may be such that the ring atoms are only carbon atoms or may include carbon and non-carbon atoms (see Heteroaryl).

“Aryl” means a monocyclic or polycyclic ring assembly where all the ring atoms are carbon atoms, and at least one of the rings comprising the ring assembly is an aromatic ring. If one or more ring atoms is not carbon (e.g., N, S), the ring assembly is a heteroaryl. C_{X} aryl and C_{X-Y} aryl are typically used where X and Y indicate the number of carbon atoms in the ring.

“Azaalkyl” means an alkyl, as defined above, except where one or more substituted or unsubstituted nitrogen atoms (—N—) are positioned between carbon atoms of the alkyl. For example, an (C₂₋₆) azaalkyl refers to a chain comprising between 2 and 6 carbons and one or more nitrogen atoms positioned between the carbon atoms.

“Bicyclic” means a two-ringed ring assembly where the two rings are fused together, linked by a single bond or linked by two bridging atoms.

“Bicycloalkyl” means a saturated or partially unsaturated fused bicyclic or bridged polycyclic ring assembly.

“Bicycloaryl” means a ring assembly of two rings, wherein the rings are linked by a single bond or fused and at least one of the rings comprising the ring assembly is an aromatic ring. C_{X} bicycloaryl and C_{X-Y} bicycloaryl are typically used where X and Y indicate the number of carbon atoms in the bicyclic ring assembly and directly attached to the ring.

“Bridging ring” as used herein refers to a ring that is bonded to another ring to form a compound having a bicyclic structure where two ring atoms that are common to both rings are not directly bound to each other. Non-exclusive examples of common compounds having a bridging ring include borneol, norbornane, 7-oxabicyclo[2.2.1]heptane, and the like. One or both rings of the bicyclic system may also comprise heteroatoms.

“Carbamoyl” means the radical —OC(O)NRₐR_{b} where Rₐ and R_{b} are each independently hydrogen or a non-hydrogen substituent.

“Carbocycle” means a ring consisting of carbon atoms.

“Carbocyclic ketone derivative” means a carbocyclic derivative wherein the ring contains a —C(═O)— moiety.

“Carbonyl” typically means a divalent radical —C(═O)—. It is noted that the term “carbonyl” when referring to a monovalent substituent can alternatively refer to a substituted carbonyl or acyl group, —C(═O)Rₐ, where Rₐ is hydrogen or a non-hydrogen substituent on the carbonyl carbon, forming different carbonyl-containing groups including acids, acid halides, aldehydes, amides, esters, and ketones.

“Carboxy” typically means a divalent radical —C(O)O—. It is noted that the term “carboxy” when referring to a monovalent substituent means a substituted carboxy, —C(O)ORₐ, where Rₐ is hydrogen or a non-hydrogen substituent on the carboxyl group forming different carboxy containing groups including acids and esters. It is further noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Cycloalkyl” means a radical comprising a non-aromatic, saturated or partially unsaturated, monocyclic, fused or bridged polycyclic ring assembly. C_{X} cycloalkyl and C_{X-Y} cycloalkyl are typically used where X and Y indicate the number of carbon atoms in the ring assembly. For example, C₃₋₁₀ cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like.

“Cycloalkylene” means a divalent radical comprising a saturated or partially unsaturated, monocyclic or polycyclic ring assembly. C_{X} cycloalkylene and C_{X-Y} cycloalkylene are typically used where X and Y indicate the number of carbon atoms in the ring assembly.

“Disease” specifically includes any unhealthy condition of an animal or part thereof and includes an unhealthy condition that may be caused by, or incident to, medical or veterinary therapy applied to that animal, i.e., the “side effects” of such therapy.

“Fused ring” as used herein refers to a multi-ring assembly wherein the rings comprising the ring assembly are so linked that the ring atoms that are common to two rings are directly bound to each other. The fused ring assemblies may be saturated, partially saturated, carbocyclics, heterocyclics, aromatics, heteroaromatics, and the like. Non-exclusive examples of common fused rings include decalin, naphthalene, anthracene, phenanthrene, indole, benzofuran, purine, quinoline, and the like.

“Halo” means fluoro, chloro, bromo or iodo.

“Halo-substituted alkyl”, as an isolated group or part of a larger group, means “alkyl” substituted by one or more “halo” atoms, as such terms are defined in this Application. Halo-substituted alkyl includes haloalkyl, dihaloalkyl, trihaloalkyl, perhaloalkyl and the like (e.g., halo-substituted (C₁₋₃)alkyl includes chloromethyl, dichloromethyl, difluoromethyl, trifluoromethyl, 2,2,2-trifluoroethyl, perfluoroethyl, 2,2,2-trifluoro-1,1-dichloroethyl, and the like).

“Heteroalkyl” means alkyl, as defined in this Application, provided that one or more of the atoms within the alkyl chain is a heteroatom.

“Heteroaryl” means a monocyclic or polycyclic ring assembly wherein at least one ring atom is a heteroatom and the remaining ring atoms are carbon, and at least one of the rings comprising the ring assembly is an aromatic ring. Monocyclic heteroaryl groups include, but are not limited to, cyclic aromatic groups having five or six ring atoms, wherein at least one ring atom is a heteroatom and the remaining ring atoms are carbon. The nitrogen atoms of such heteroaryl rings can be optionally quaternerized and the sulfur atoms of such heteroaryl rings can be optionally oxidized. Heteroaryl groups of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrroline, thiazole, 1,3,4-thiadiazole, triazole and tetrazole. “Heteroaryl” also includes polycyclic ring assemblies, wherein a heteroaromatic ring is fused or linked by a bond to one or more rings independently selected from the group consisting of an aromatic ring, a cycloalkyl ring, a cycloalkenyl ring, a heterocycloalkyl ring and another heteroaromatic ring. Bicyclic or tricyclic heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[ 1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole and 2(1H)-pyridinone. The polycyclic heteroaryl ring assembly can be attached to the parent molecule through either the heteroaryl group itself or the aryl, cycloalkyl, cycloalkenyl or heterocycloalkyl group to which it is fused. The heteroaryl groups of this invention can be substituted or unsubstituted.

“Heterobicycloaryl” means bicycloaryl, as defined in this Application, provided that one or more of the atoms within the ring assembly is a heteroatom. For example, hetero(C₄₋₁₂)bicycloaryl as used in this Application includes, but is not limited to, indoline, 2-amino-4-oxo-3,4-dihydropteridin-6-yl, tetrahydroisoquinolinyl, and the like.

“Heterocycloalkyl” means cycloalkyl, as defined in this Application, provided that one or more of the atoms forming the ring is a heteroatom. Non-exclusive examples of heterocycloalkyl include piperidyl, 4-morpholyl, 4-piperazinyl, pyrrolidinyl, perhydropyrrolizinyl, 1,3-dioxanyl, 1,4-dioxanyl and the like.

“Heteroatom” refers to an atom that is not a carbon atom. Particular examples of heteroatoms include, but are not limited to nitrogen, oxygen, and sulfur.

“Heteroatom moiety” includes a moiety where the atom by which the moiety is attached is not a carbon. Examples of heteroatom moieties include —N═, —NR—, —N⁺(O⁻)═, —O—, —S— or —S(O)₂—, wherein R is hydrogen or a non-hydrogen substituent.

“Heterobicycloalkyl” means bicycloalkyl, as defined in this Application, provided that one or more of the atoms within the ring is a heteroatom. For example hetero(C₉₋₂)bicycloalkyl as used in this application includes, but is not limited to, 3-aza-bicyclo[4.1.0]hept-3-yl, 2-aza -bicyclo[3.1. O]hex-2-yl, 3-aza-bicyclo[3.1.0]hex-3-yl, and the like.

“Heterocycle” refers to a ring moiety, saturated, unsaturated or aromatic, where at least one ring atom is a heteroatom and the remaining ring atoms are carbon.

“Heterocycloalkylene” means cycloalkylene, as defined in this Application, provided that one or more of the ring member carbon atoms is replaced by a heteroatom.

“Hydroxy” means the radical —OH.

“IC₅₀” referes to the molar concentration of an inhibitor that produces 50% inhibition of the target enzyme.

“Iminoketone derivative” means a derivative comprising the moiety —C(═NR)—, wherein R is hydrogen or a non-hydrogen substituent attached to the nitrogen.

“Isomers” mean any compounds having identical molecular formulae but differing in the nature or sequence of bonding of their atoms or in the arrangement of their atoms in space. Isomers that differ in the arrangement of their atoms in space are termed “stereoisomers.” Stereoisomers that are not mirror images of one another are termed “diastereomers” and stereoisomers that are nonsuperimposable mirror images are termed “enantiomers” or sometimes “optical isomers.” A carbon atom bonded to four different substituents (where no two are the same) is termed a “chiral center.” A compound with one chiral center has two enantiomeric forms of opposite chirality. A mixture of equal amounts of the two enantiomeric forms is termed a “racemic mixture.” A compound that has more than one chiral center has 2ⁿ⁻¹ enantiomeric pairs, where n is the number of chiral centers. Compounds with more than one chiral center may exist as ether an individual diastereomer or as a mixture of diastereomers, termed a “diastereomeric mixture.” When one chiral center is present a stereoisomer may be characterized by the absolute configuration of that chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

“Moiety” means an interconnected group of atoms, generally referred to by its most characteristic structural component. For example, a “carbonyl moiety” refers to groups that contain a carbonyl group.

“Nitro” means the radical —NO₂.

“Oxaalkyl” means an alkyl, as defined above, except where one or more oxygen atoms (—O—) are positioned between carbon atoms of the alkyl. For example, an (C₂₋₆)oxaalkyl refers to a chain comprising between 2 and 6 carbons wherein one or more oxygen atoms is positioned between two carbon atoms.

“Oxy” typically means the radical —O—. It is noted that the term “oxy” when referring to a monovalent radical can alternatively refer to a substituents oxy group, —OR—, where R is hydrogen or a non-hydrogen substituent on the oxy radical forming oxy-containing groups including hydroxy, alkoxy, aryloxy, heteroaryloxy and carbonyloxy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Prodrug” means a compound that is convertible in vivo metabolically into an inhibitor according to the present invention. The prodrug itself may or may not also have Renin inhibitory activity. For example, an inhibitor comprising a hydroxy group may be administered as an ester that is converted by hydrolysis in vivo to the hydroxy compound. Suitable esters that may be converted in vivo into hydroxy compounds include acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, an inhibitor comprising an amine group may be administered as an amide or as an N-alkyl (particularly N-methyl or N-ethyl) that is converted by hydrolysis or oxidation in vivo to the amine compound.

“Protected derivatives” means derivatives of inhibitors in which a reactive site or sites are blocked with protecting groups. Protected derivatives are useful in the preparation of inhibitors or in themselves may be active as inhibitors. Examples of protected group includes, but are not limited to, acetyl, tetrahydropyran, methoxymethyl ether, β-methoxyethoxymethyl ether, p-methoxybenzyl, methylthiomethyl ether, pivaloyl, silyl ether, carbobenzyloxy, benzyl, tert-butoxycarbonyl, ρ-methoxyphenyl, 9-fluorenylmethyloxycarbonyl, acetals, ketals, acylals, dithianes, methylesters, benzyl esters, tert-butyl esters, and silyl esters. A comprehensive list of suitable protecting groups can be found in T. W. Greene, Protecting Groups in Organic Synthesis, 3rd edition, John Wiley & Sons, Inc. 1999.

“Ring” means a carbocyclic or a heterocyclic system.

“Substituent convertible to hydrogen in vivo” means any group that is convertible to a hydrogen atom by enzymological or chemical means including, but not limited to, hydrolysis, reduction and oxidation. Examples include hydrolyzable groups, such as acyl groups, groups having an oxycarbonyl group, amino acid residues, peptide residues, o-nitrophenylsulfenyl, trimethylsilyl, tetrahydro-pyranyl, diphenylphosphinyl, and the like. Examples of acyl groups include formyl, acetyl, trifluoroacetyl, and the like. Examples of groups having an oxycarbonyl group include ethoxycarbonyl, t-butoxycarbonyl [—(O)CO—C(CH₃)₃], benzyloxycarbonyl, p-methoxybenzyloxycarbonyl, vinyloxycarbonyl, β-(p-toluenesulfonyl)ethoxycarbonyl, and the like. Examples of suitable amino acid residues include amino acid residues per se and amino acid residues that are protected with a protecting group. Suitable amino acid residues include, but are not limited to, residues of Gly (glycine), Ala (alanine; —C(O)CH(NH2)CH₃), Arg (arginine), Asn (asparagine), Asp (aspartic acid), Cys (cysteine), Glu (glutamic acid), His (histidine), Ile (isoleucine), Leu (leucine; —C(O)CH(NH₂)CH₂CH(CH₃)₂ Lys (lysine), Met (methionine), Phe (phenylalanine), Pro (proline), Ser (serine), Thr (threonine), Trp (tryptophan), Tyr (tyrosine), Val (valine), Nva (norvaline), Hse (homoserine), 4-Hyp (4-hydroxyproline), 5-Hyl (5-hydroxylysine), Om (omithine) and β-Ala. Examples of suitable protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [—(O)CO—C(CH₃)₃], and the like. Suitable peptide residues include petide residues comprising two to five, and optionally two to three, of the aforesaid amino acid residues. Examples of such peptide residues include, but are not limited to, residues of such peptides as Ala-Ala [—C(O)CH(NH)CH₃—C(O)CH(NH₂)CH₃)], Gly-Phe, Nva-Nva, Ala-Phe, Gly-Gly, Gly-Gly-Gly, Ala-Met, Met-Met, Leu-Met and Ala-Leu. The residues of these amino acids or peptides can be present in stereochemical configurations of the D-form, the L-form or mixtures thereof. In addition, the amino acid or peptide residue may have an asymmetric carbon atom. Examples of suitable amino acid residues having an asymmetric carbon atom include residues of Ala, Leu, Phe, Trp, Nva, Val, Met, Ser, Lys, Thr and Tyr. Peptide residues having an asymmetric carbon atom include peptide residues having one or more constituent amino acid residues having an asymmetric carbon atom. Examples of suitable amino acid protecting groups include those typically employed in peptide synthesis, including acyl groups (such as formyl and acetyl), arylmethyloxycarbonyl groups (such as benzyloxycarbonyl and p-nitrobenzyloxycarbonyl), t-butoxycarbonyl groups [—(O)CO—C(CH₃)₃], and the like. Other examples of substituents “convertible to hydrogen in vivo” include reductively eliminable hydrogenolyzable groups. Examples of suitable reductively eliminable hydrogenolyzable groups include, but are not limited to, arylsulfonyl groups (such as o-toluenesulfonyl); methyl groups substituted with phenyl or benzyloxy (such as benzyl, trityl and benzyloxymethyl); arylmethoxycarbonyl groups (such as benzyloxycarbonyl and o-methoxy-benzyloxycarbonyl); and halogenoethoxycarbonyl groups (such as β,β,β-trichloroethoxycarbonyl and β-iodoethoxycarbonyl). Further examples of substituents “convertible to hydrogen in vivo” include enzymatic oxidizable groups such as N-alkyls, particularly N-methyl and N-ethyl.

“Substituted or unsubstituted” or “optionally substituted” means that a given moiety may consist of only hydrogen atoms bound at available valences (unsubstituted) or may further comprise one or more non-hydrogen atoms bound through available valencies (substituted). The substituents of an “optionally substituted” group may include, without limitationone or more substituents independently selected from the group or designated subsets thereof, aldehyde, (C₁₋₁₀)alkyl, alkylene, alkylidene, amide, amino, aminoalkyl, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl, and oxoalkyl moieties, each of which may optionally also be substituted or unsubstituted.

In one particular embodiment, examples of substituents include, but are not limited to, hydrogen, halo, nitro, cyano, thio, oxy, hydroxy, carbonyloxy, (C₁₋₁₀)alkoxy, (C₄₋₁₄)aryloxy, hetero(C₁₋₁₃)aryloxy, carbonyl, oxycarbonyl, aminocarbonyl, amino, (C₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl. In addition, the substituent is itself optionally substituted by a further substituent. In one particular embodiment, examples of the further substituent include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl.

“Sulfinyl” means the radical —S(O)—. It is noted that the term “sulfinyl” when referring to a monovalent subsituent can alternatively refer to a substituted sulfinyl group, —S(═O)R, where R is hydrogen or a non-hydrogen substituent on the sulfur atom forming different sulfinyl groups including sulfinic acids, sulfinamides, sulfinyl esters, and sulfoxides.

“Sulfonyl” means the radical —S(O)₂—. It is noted that the term “sulfonyl” when referring to a monovalent subsituent can alternatively refer to a substituted sulfonyl group, —S(═O)₂R, where R is hydrogen or a non-hydrogen substituent on the sulfur atom forming different sulfonyl groups including sulfonic acids, sulfonamides, sulfonate esters, and sulfones.

“Therapeutically effective amount” means that amount which, when administered to an animal for treating a disease, is sufficient to effect such treatment for the disease.

“Thiocarbonyl” means the radical —C(S)—. It is noted that the term thiocarbonyl when referring to a monovalent subsituent can alternatively refer to a substituted thiocarbonyl group, —C(═S)₂R, where R is hydrogen or a non-hydrogen substituent on the carbon atom forming different thiocarbonyl groups including thioacids, thioamides, thioesters, and thioketones.

“Treatment” or “treating” means any administration of a compound of the present invention and includes:
(1) preventing the disease from occurring in an animal which may be predisposed to the disease but does not yet experience or display the pathology or symptomatology of the disease,
(2) inhibiting the disease in an animal that is experiencing or displaying the pathology or symptomatology of the diseased (i.e., arresting further development of the pathology and/or symptomatology), or
(3) ameliorating the disease in an animal that is experiencing or displaying the pathology or symptomatology of the diseased (i.e., reversing the pathology and/or symptomatology).

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} may each independently be hydrogen or any other substituent where the atom attached to the carbon is not a hydrogen atom. Hence, —CF₃, —CH₂OH and —CH₂CN, for example, are all C₁ alkyls.

### DETAILED DESCRIPTION OF THE INVENTION

The present invention relates to compounds, compositions, kits and articles of manufacture that may be used to inhibit histone deacetylases (HDACs) and, in particular, Class I HDACs such as HDAC1, HDAC2, HDAC6 and HDAC8.

At least seventeen human genes that encode proven or putative HDACs have been identified to date, some of which are described in Johnstone, R. W., “Histone-Deacetylase Inhibitors: Novel Drugs for the Treatment of Cancer”, Nature Reviews, Volume I, pp. 287-299, (2002) and PCT Publication Nos. 00/10583, 01/18045, 01/42437 and 02/08273.

HDACs have been categorized into three distinct classes based on their relative size and sequence homology. The different HDACs (Homo sapiens), HDAC classes, sequences and references describing the different HDACs are provided in Tables 1-3.

TABLE 1
CLASS I HDACs

| | GenBank | |
|---|---|---|
| HDAC | Accession Number | Reference |
| 1 | NP_004955 | Histone deacetylase: a regulator of transcription, |
| | | Wolffe, A. P., Science 272 (5260), 371-372 (1996) |
| 2 | NP_001518 | Isolation and mapping of a human gene (RPD3L1) that |
| | | is homologous to RPD3, a transcription factor in |
| | | Saccharomyces cerevisiae; Furukawa, Y., |
| | | Kawakami, T., Sudo, K., Inazawa, J., Matsumine, A., |
| | | Akiyama, T. and Nakamura, Y., Cytogenet. Cell Genet. |
| | | 73 (1-2), 130-133 (1996) |
| 3 | NP_003874 | Isolation and characterization of cDNAs corresponding |
| | | to an additional member of the human histone |
| | | deacetylase gene family, Yang, W. M., Yao, Y. L., |
| | | Sun, J. M., Davie, J. R. and Seto, E., J. Biol. Chem. 272 |
| | | (44), 28001-28007 (1997) |
| 8 | NP_060956 | Buggy, J. J., Sideris, M. L., Mak, P., Lorimer, D. D., |
| | | McIntosh, B. and Clark, J. M. |
| | | Biochem. J. 350 Pt 1, 199-205 (2000) |
| 11 | NP_079103 | Cloning and Functional Characterization of HDAC11, |
| | | a Novel Member of the Human Histone Deacetylase |
| | | Family, Gao, L., Cueto, M. A., Asselbergs, F. and |
| | | Atadja, P., J. Biol. Chem. 277 (28), 25748-25755 |
| | | (2002) |

TABLE 2
CLASS II HDACs

| | GenBank | |
|---|---|---|
| HDAC | Accession Number | Reference |
| 4 | NP_006028 | Transcriptional control. Sinful repression, Wolffe, A. P., |
| | | Nature 387 (6628), 16-17 (1997) |
| 5 | NP_631944 | Prediction of the coding sequences of unidentified |
| | | human genes. IX. The complete sequences of 100 new |
| | | cDNA clones from brain which can code for large |
| | | proteins in vitro, Nagase, T., Ishikawa, K., Miyajima, N., |
| | | Tanaka, A., Kotani, H., Nomura, N. and Ohara, O., DNA |
| | | Res. 5 (1), 31-39 (1998) |
| 6 | NP_006035 | Transcriptional control. Sinful repression, Wolffe, A. P., |
| | | Nature 387 (6628), 16-17 (1997) |
| 7 | NP_057680 | Isolation of a novel histone deacetylase reveals that |
| | | class I and class II deacetylases promote SMRT-mediated |
| | | repression, Kao, H. Y., Downes, M., Ordentlich, P. and |
| | | Evans, R. M., Genes Dev. 14 (1), 55-66 (2000) |
| 9 | NP_478056 | MEF-2 function is modified by a novel co-repressor, |
| | | MITR, Sparrow, D. B., Miska, E. A., Langley, E., |
| | | Reynaud-Deonauth, S., Kotecha, S., Towers, N., |
| | | Spohr, G., Kouzarides, T. and Mohun, T. J., EMBO J. 18 |
| | | (18), 5085-5098 (1999) |
| 10 | NP_114408 | Isolation and characterization of mammalian |
| | | HDAC10, a novel histone deacetylase, Kao, H. Y., |
| | | Lee, C. H., Komarov, A., Han, C. C. and Evans, R. M., J. |
| | | Biol. Chem. 277 (1), 187-193 (2002) |

TABLE 3
CLASS III HDACs

| | GenBank | |
|---|---|---|
| HDAC | Accession Number | Reference |
| Sirtuin 1 | NP_036370 | Characterization of five human cDNAs with homology |
| | | to the yeast SIR2 gene: Sir2-like proteins (sirtuins) |
| | | metabolize NAD and may have protein ADP- |
| | | ribosyltransferase activity; Frye, R. A.; Biochem. |
| | | Biophys. Res. Commun. 260 (1), 273-279 (1999) |
| Sirtuin 2 | NP_085096/ | A ‘double adaptor’ method for improved shotgun |
| | NP_036369 | library construction; Andersson, B., Wentland, M. A., |
| | | Ricafrente, J. Y., Liu, W. and Gibbs, R. A.; Anal. |
| | | Biochem. 236 (1), 107-113 (1996) |
| Sirtuin 3 | NP_036371 | Characterization of five human cDNAs with homology |
| | | to the yeast SIR2 gene: Sir2-like proteins (sirtuins) |
| | | metabolize NAD and may have protein ADP- |
| | | ribosyltransferase activity; Frye, R. A.; Biochem. |
| | | Biophys. Res. Commun. 260 (1), 273-279 (1999) |
| Sirtuin 4 | NP_036372 | Characterization of five human cDNAs with homology |
| | | to the yeast SIR2 gene: Sir2-like proteins (sirtuins) |
| | | metabolize NAD and may have protein ADP- |
| | | ribosyltransferase activity; Frye, R. A.; Biochem. |
| | | Biophys. Res. Commun. 260 (1), 273-279 (1999) |
| Sirtuin 5 | NP_112534/ | Characterization of five human cDNAs with homology |
| | NP_036373 | to the yeast SIR2 gene: Sir2-like proteins (sirtuins) |
| | | metabolize NAD and may have protein ADP- |
| | | ribosyltransferase activity; Frye, R. A.; Biochem. |
| | | Biophys. Res. Commun. 260 (1), 273-279 (1999) |
| Sirtuin 6 | NP_057623 | Phylogenetic classification of prokaryotic and |
| | | eukaryotic Sir2-like proteins; Frye, R. A.; Biochem. |
| | | Biophys. Res. Commun. 273 (2), 793-798 (2000) |
| Sirtuin 7 | NP_057622 | Phylogenetic classification of prokaryotic and |
| | | eukaryotic Sir2-like proteins; Frye, R. A.; Biochem. |
| | | Biophys. Res. Commun. 273 (2), 793-798 (2000) |

Of particular note are Class I HDACs. All Class I HDACs appear to be sensitive to inhibition by trichostatin A (TSA). Of particular note HDAC2 and HDAC8, proteins whose crystal structures Applicants determined and used in conjunction with arriving at the present invention.

HDAC2 is a 488 residue, 55 kDa protein localized to the nucleus of a wide array of tissues, as well as several human tumor cell lines. The wild-type form of full length HDAC2 is described in GenBank Accession Number NM 001527, Furukawa, Y. et al., Cryogenet. Cell Genet., 73 (1-2), 130-133 (1996). Zn²⁺ is likely native to the protein and required for HDAC2 activity.

HDAC8 is a 377 residue, 42 kDa protein localized to the nucleus of a wide array of tissues, as well as several human tumor cell lines. The wild-type form of full length HDAC8 is described in GenBank Accession Number NP 060956; Buggy, J. J. et al., Biochem. J., 350 (Pt 1), 199-205 (2000). Zn²⁺ is likely native to the protein and required for HDAC8 activity.

Crystal Structure of Histone Deacetylase

Syrrx, Inc. (now Takeda San Diego, Inc.) in San Diego, Calif. solved the crystal structure for HDAC2 (U.S. patent Ser. Nos. 10/826,134 and 10/826,170, both filed Apr. 16, 2004, each of which is hereby incorporated by reference in its entirety) and HDAC8 (U.S. patent Ser. Nos. 10/601,058 and 10/601,335, both filed Jun. 20, 2003, each of which is hereby incorporated by reference in its entirety).

HDAC2 was found to adopt an open-faced α/β structure consisting of 8 central parallel 1-sheets sandwiched between 12 α-helices. The ligand binding cleft lies almost in the plane of the central β-sheet, and is formed primarily by loops emanating from the carboxy-terminal ends of the β-strands comprising the sheet. Residues which form loop regions extending between β-strand 1 and α-helix 1 and between α-helix 4 and α-helix 5, provide key surface interactions with bound ligands. Residues which form loop regions extending between β-strand 3 and α-helix 6 and between β-strand 4 and α-helix 7 and between β-strand 8 and α-helix 10 play important roles in defining the shape of the ligand binding pocket, and are involved in a number of key interactions with the bound ligands.

HDAC8 was found to have a single domain structure belonging to the open α/β class of folds. The structure consists of a central 8-stranded parallel β-sheet sandwiched between layers of α-helices. The ligand binding clefts lie almost in the plane of the central β-sheet, and are formed primarily by loops emanating from the carboxy-terminal ends of the β-strands comprising the sheet. There are two large structural extensions, which occur beyond the core of the α/β motif, off the second and last β-strands of the central β-sheet. Residues contained in the extension off the second β-strand form a globular “cap” over the core of the protein, play an important role in defining the shape of the ligand binding pockets, and are involved in a number of key interactions with the bound ligands.

Knowledge of the crystal structures was used to guide the design of the HDAC inhibitors provided herein.

Histone Deacetylase Inhibitors

In one embodiment, HDAC inhibitors of the present invention comprise: 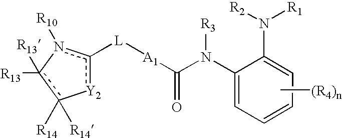
wherein:
n is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L is a linker comprising a backbone chain of 1 to 10 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond; and
R₁₃, R₁₃′, R₁₄, and R₁₄′ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃-1₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted, or R₁₃ and R₁₄ are taken together to form a ring, or R₁₃′ and R₁₄′ are absent when the atoms to which they are bound form part of a double bond.

In particular variations of the above embodiment, (a) Y₂ is not S when R₁, R₂, R₃, R₁₀, R₁₃′, R₁₄, and R₁₄′ are all H; A₁ is phenylene; L is methylene; and R₁₃ is morpholino-4-ylmethyl; (b) L is not —S—CH₂— when R₁₃ and R₁₄ form a fused phenyl ring; Y₂ is NH; and R₁, R₂, and R₃ are all H; (c) L is not —O—CH₂— when R₁₃ and R₁₄ form a fused phenyl ring; Y₂ is O; and R₁, R₂, and R₃ are all H; (d) L is not —NH—CH₂— when R₁₃ and R₁₄ form a fused phenyl ring; Y₂ is S; and R₁, R₂, and R₃ are all H; (e) L is not —NH—CH₂—CH═CH— when R₁₃ and R₁₄ form a fused phenyl ring; Y₂ is S; and R₁, R₂, and R₃ are all H; and (f) when R₁₃ and R₁₃′ are taken together with the 5-membered ring to which they are attached to form benzimidazole; A₁ is 1,4-phenylene; and L is methylene; R₆ is not 5-F or 5-MeO when p is 1; and R₆ is not 5-Cl, 6-F or 5-F, 6-Cl when p is 2.

In anther embodiment HDAC inhibitors of the present invention comprise: 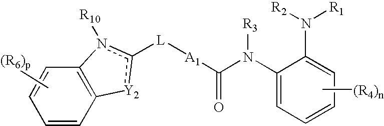
wherein:
n is 0, 1, 2, 3, or 4;
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L is a linker comprising a backbone chain of 1 to 10 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₁₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

In particular variations of the above embodiment, (a) Y₂ is not S when R₁, R₂, R₃, R₁₀, R₁₃′, R₁₄, and R₁₄′ are all H; A₁ is phenylene; L is methylene; and R₁₃ is morpholino-4-ylmethyl; (b) L is not —S—CH₂— when R₁₃ and R₁₄ form a fused phenyl ring; Y₂ is NH; and R₁, R₂, and R₃ are all H; (c) L is not —O—CH₂— when R₁₃ and R₁₄ form a fused phenyl ring; Y2 is O; and R₁, R₂, and R₃ are all H; (d) L is not —NH—CH₂— when R₁₃ and R₁₄ form a fused phenyl ring; Y₂ is S; and R₁, R₂, and R₃ are all H; (e) L is not —NH—CH₂—CH═CH— when R₁₃ and R₁₄ form a fused phenyl ring; Y₂ is S; and R₁, R₂, and R₃ are all H; and (f) when A₁ is 1,4-phenylene; L is methylene; R₁₀ is absent and Y₂ is NH, R₆ is not 5-F or 5-MeO when p is 1 and R₆ is not 5-Cl, 6-F or 5-F, 6-Cl when p is 2.

In other variations of the above embodiment, (a) when Y₂ is NH; and R₁, R₂, and R₃ are all H; L is not —S—CH₂—; (b) when Y₂ is O; and R₁, R₂, and R₃ are all H; L is not —O—CH₂—; (c) when Y₂ is S; and R₁, R₂, and R₃ are all H; L is not —NH—CH₂—; (d) when Y₂ is S; and R₁, R₂, and R₃ are all H; L is not —NH—CH₂—CH═CH—; (e) when A₁ is 1,4-phenylene; L is methylene; R₁₀ is absent, Y₂ is NH, and p is 1; R₆ is not 5-F or 5-MeO; and (f) when A₁ is 1,4-phenylene; L is methylene; R₁₀ is absent; Y₂ is NH; and p is 2; R₆ is not 5-Cl, 6-F or 5-F, 6-Cl.

In a further embodiment, HDAC inhibitors of the present invention comprise: 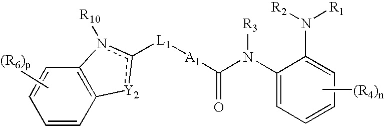
wherein:
n is 0, 1, 2, 3, or 4;
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L₁ is selected from the group consisting of (C₁₋₆)alkylene, (C₂₋₆)alkenylene, and (C₂₋₆)alkynylene, each substituted or unsubstituted;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted.

In particular variations of the above embodiment, when A₁ is 1,4-phenylene; L₁ is methylene; R₁₀ is absent; and Y₂ is NH; R₆ is not 5-F or 5-MeO when p is 1; and R₆ is not 5-Cl, 6-F or 5-F, 6-Cl when p is 2.

In yet another embodiment, HDAC inhibitors of the present invention comprise: 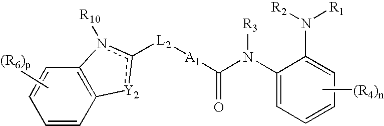
wherein:
n is 0, 1, 2, 3, or 4;
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L₂ is selected from the group consisting of CR₁₅R₁₆, NR₁₇, S, S(O) and S(O)₂;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted.

In particular variations of the above embodiment, when A₁ is 1,4-phenylene; L₂ is methylene; R₁₀ is absent; and Y₂ is NH; R₆ is not 5-F or 5-MeO when p is 1; and R₆ is not 5-Cl, 6-F or 5-F, 6-Cl when p is 2.

In yet another embodiment, HDAC inhibitors of the present invention comprise: 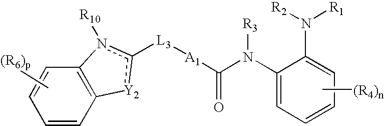
wherein:
n is 0, 1, 2, 3, or 4;
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L₃ is selected from the group consisting of *CR₁₅R₁₆, *OCR₁₅R₁₆, *NR₁₇CR₁₅R₁₆, *SCR₁₅R₁₆, *S(O)CR₁₅R₁₆ and *S(O)₂CR₁₅R₁₆, where * indicates the point of attachment of L₃ to the five membered ring containing Y₂;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted.

In particular variations of the above embodiment, when A₁ is 1,4-phenylene; L₃ is methylene; R₁₀ is absent; and Y₂ is NH; R₆ is not 5-F or 5-MeO when p is 1; and R₆ is not 5-Cl, 6-F or 5-F, 6-Cl when p is 2.

In still another embodiment, HDAC inhibitors of the present invention comprise: 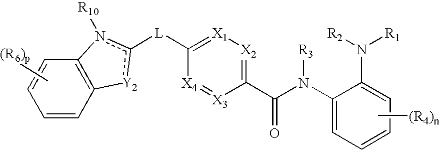
wherein:
n is 0, 1, 2, 3, or 4;
p is 0, 1, 2, 3, or 4;
L is a linker comprising a backbone chain of 1 to 10 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
X₁, X₂, X₃, and X₄ are each independently selected from the group consisting of CR₅ and N;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or Rio is absent when the nitrogen to which it is bound forms part of a double bond.

In particular variations of the above embodiment, (a) Y₂ is not S when R₁, R₂, R₃, R₁₀, R₁₃′, R₁₄, and R₁₄′ are all H; X₁, X₂, X₃, and X₄ are all CH; L is methylene; and R₁₃ is morpholino-4-ylmethyl; (b) L is not —S—CH₂— when R₁₃ and R₁₄ form a fused phenyl ring; Y₂ is NH; X₁, X₂, X₃, and X₄ are all CH; and R₁, R₂, and R₃ are all H; (c) L is not —O—CH₂— when R₁₃ and R₁₄ form a fused phenyl ring; Y₂ is O; X₁, X₂, X₃, and X₄ are all CH; and R₁, R₂, and R₃ are all H; (d) L is not —NH—CH₂— when R₁₃ and R₁₄ form a fused phenyl ring; Y₂ is S; X₁, X₂, X₃, and X₄ are all CH; and R₁, R₂, and R₃ are all H; (e) L is not —NH—CH₂—CH═CH— when R₁₃ and R₁₄ form a fused phenyl ring; Y₂ is S; X₁, X₂, X₃, and X₄ are all CH; and R₁, R₂, and R₃ are all H; and (f) when X₁, X₂, X₃ and X₄ are all CH; L is methylene; R₁₀ is absent; and Y₂ is NH; R₆ is not 5-F or 5-MeO when p is 1; and R₆ is not 5-Cl, 6-F or 5-F, 6-Cl when p is 2.

In other variations of the above embodiment, (a) when Y₂ is NH; X₁, X₂, X₃, and X₄ are all CH; and R₁, R₂, and R₃ are all H; L is not —S—CH₂—; (b) when Y₂ is O; X₁, X₂, X₃, and X₄ are all CH; and R₁, R₂, and R₃ are all H; L is not —O—CH₂—; (c) when Y₂ is S; X₁, X₂, X₃, and X₄ are all CH; and R₁, R₂, and R₃ are all H; L is not —NH—CH₂—; (d) when Y₂ is S; X₁, X₂, X₃, and X₄ are all CH; and R₁, R₂, and R₃ are all H; L is not —NH—CH₂—CH═CH—; (e) when X₁, X₂, X₃ and X₄ are all CH; L is methylene; R₁₀ is absent; Y₂ is NH; and p is 1; R₆ is not 5-F or 5-MeO; and (f) when X₁, X₂, X₃ and X₄ are all CH; L is methylene; R₁₀ is absent; and Y₂ is NH; and p is 2; R₆ is not 5-Cl, 6-F or 5-F, 6-Cl.

In another embodiment, HDAC inhibitors of the present invention comprise: 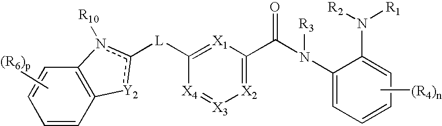
wherein:
n is 0, 1, 2, 3, or 4;
p is 0, 1, 2, 3, or 4;
L is a linker comprising a backbone chain of 1 to 10 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
X₁, X₂, X₃, and X₄ are each independently selected from the group consisting of CR₅ and N;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₃₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

In particular variations of the above embodiment, (a) Y₂ is not S when R₁, R₂, R₃, R₁₀, R₁₃′, R₁₄, and R₁₄′ are all H; X₁, X₂, X₃, and X₄ are all CH; L is methylene; and R₁₃ is morpholino-4-ylmethyl; (b) L is not —S—CH₂— when R₁₃ and R₁₄ form a fused phenyl ring; Y₂ is NH; X₁, X₂, X₃, and X₄ are all CH; and R₁, R₂, and R₃ are all H; (c) L is not —O—CH₂— when R₁₃ and R₁₄ form a fused phenyl ring; Y₂ is O; X₁, X₂, X₃, and X₄ are all CH; and R₁, R₂, and R₃ are all H; (d) L is not —NH—CH₂— when R₁₃ and R₁₄ form a fused phenyl ring; Y₂ is S; X₁, X₂, X₃, and X₄ are all CH; and R₁, R₂, and R₃ are all H; and/or (e) L is not —NH—CH₂—CH═CH— when R₁₃ and R₁₄ form a fused phenyl ring; Y₂ is S; X₁, X₂, X₃, and X₄ are all CH; and R₁, R₂, and R₃ are all H.

In other variations of the above embodiment, (a) when Y₂ is NH; X₁, X₂, X₃, and X₄ are all CH; and R₁, R₂, and R₃ are all H; L is not —S—CH₂—; (b) when Y₂ is O; X₁, X₂, X₃, and X₄ are all CH; and R₁, R₂, and R₃ are all H; L is not —O—CH₂—; (c) when Y₂ is S; X₁, X₂, X₃, and X₄ are all CH; and R₁, R₂, and R₃ are all H; L is not —NH—CH₂—; and/or (d) when Y₂ is S; X₁, X₂, X₃, and X₄ are all CH; and R₁, R₂, and R₃ are all H; L is not —NH—CH₂—CH═CH—.

In a further embodiment, HDAC inhibitors of the present invention comprise: 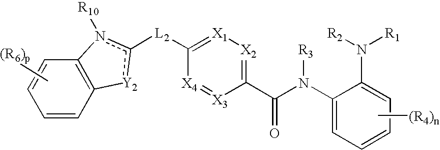
wherein:
n is 0, 1, 2, 3, or 4;
p is 0, 1, 2, 3, or 4;
L₂ is selected from the group consisting of CR₁₅R₁₆, NR₁₇, S, S(O) and S(O)₂;
X₁, X₂, X₃, and X₄ are each independently selected from the group consisting of CR₅ and N;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted.

In particular variations of the above embodiment, when X₁, X₂, X₃ and X₄ are all CH; L₂ is methylene; R₁₀ is absent; and Y₂ is NH; p is 1, R₆ is not 5-F or when X₁, X₂, X₃ and X₄ are all CH; L₂ is methylene; R₁₀ is absent; and Y₂ is NH; p is 2; R₆ is not 5-Cl, 6-F or 5-F, 6-Cl.

In a further embodiment, HDAC inhibitors of the present invention comprise: 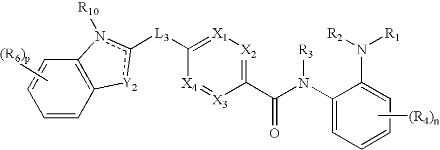
wherein:
n is 0, 1, 2, 3, or 4;
p is 0, 1, 2, 3, or 4;
L₃ is selected from the group consisting of *CR₁₅R₁₆, *OCR₁₅R₁₆, *NR₁₇CR₁₅R₁₆, *SCR₁₅R₁₆, *S(O)CR₁₅R₁₆, and *S(O)₂CR₁₅R₁₆, where * indicates the point of attachment of L₃ to the ring containing Y₂;
X₁, X₂, X₃, and X₄ are each independently selected from the group consisting of CR₅ and N;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted.

In particular variations of the above embodiment, when X₁, X₂, X₃ and X₄ are all CH; L₃ is methylene; R₁₀ is absent; and Y₂ is NH; p is 1; R₆ is not 5-F or when X₁, X₂, X₃ and X₄ are all CH; L₂ is methylene; R₁₀ is absent; and Y₂ is NH; p is 2, R₆ is not 5-Cl, 6-F or 5-F, 6-Cl.

In yet another embodiment, HDAC inhibitors of the present invention comprise: 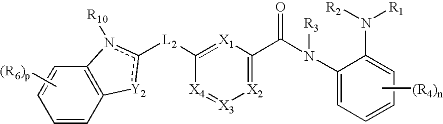
wherein:
n is 0, 1, 2, 3, or 4;
p is 0, 1, 2, 3, or 4;
L₂ is selected from the group consisting of CR₁₅R₁₆, NR₁₇, S, S(O) and S(O)₂;
X₁, X₂, X₃, and X₄ are each independently selected from the group consisting of CR₅ and N;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted.

In yet another embodiment, HDAC inhibitors of the present invention comprise: 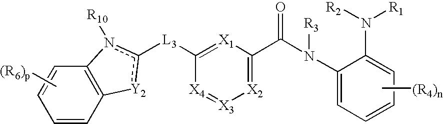
wherein:
n is 0, 1, 2, 3, or 4;
p is 0, 1, 2, 3, or 4;
L₃ is selected from the group consisting of *CR₁₅R₁₆, *OCR₁₅R₁₆, *NR₁₇CR₁₅R₁₆, *SCR₁₅R₁₆, *S(O)CR₁₅R₁₆, and *S(O)₂CR₁₅R₁₆, where * indicates the point of attachment of L₃ to the ring containing Y₂;
X₁, X₂, X₃, and X₄ are each independently selected from the group consisting of CR₅ and N;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted.

In one variation of each of the above embodiments, L is selected from the group consisting of (C₁₋₆)alkylene, (C₂₋₆)alkenylene, and (C₂₋₆)alkynylene, each substituted or unsubstituted. In another variation, L is an unsubstituted or substituted (C₁₋₄)alkylene. In a further variation, L is a (C₁₋₃)alkylene, unsubstituted or substituted. In yet another variation, L is an unsubstituted or substituted methylene.

In one variation of each of the above embodiments and variations, at least one of R₁ and R₂ is H. In a particular variation, R₁ and R₂ are both H.

In another variation of each of the above embodiments and variations, R₃ is H.

In a further variation of each of the above embodiments and variations, R₄ is hydrogen, halo, perhalo(C₁₋₃)alkyl, amino, cyano, nitro, thio, (C₁₋₅)alkyl, (C₃₋₅)cycloalkyl, hetero(C₃₋₅)cycloalkyl, aryl(C₁₋₃)alkyl, heteroaryl (C₁₋₃)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, alkenyl, alkynyl, carbonyl, and imino, each substituted or unsubstituted.

In another variation of each of the above embodiments and variations, at least one of R₁₅ and R₁₆ is H. In another variation, both R₁₅ and R₁₆ are H.

In yet another variation of each of the above embodiments and variations, A₁ is phenylene. In a particular variation, A₁ is a 1,4-phenylene.

In still another variation of each of the above embodiments and variations, Y₂ is CR₇R₈, or NR₉ where R₉ is not H. In another variation, Y₂ is CR₇R₈, or NR₉ where R₉ is not H.

Particular examples of HDAC inhibitors according to the present invention include, but are not limited to:
N-(2-aminophenyl)-4-((benzo[d]thiazol-2-ylamino)methyl)benzamide;
4-((1H-benzo[d]imidazol-2-ylamino)methyl)-N-(2-aminophenyl)benzamide;
4-((1H-benzo[d]imidazol-2-ylthio)methyl)-N-(2-aminophenyl)benzamide;
4-((3H-imidazo[4,5-b]pyridin-2-yloxy)methyl)-N-(2-aminophenyl)benzamide;
N-(2-aminophenyl)-4-((1-methyl-1H-benzo[d]imidazol-2-ylamino)methyl)benzamide;
N-(2-aminophenyl)-4-((1-methyl-1H-benzo[d]imidazol-2-yloxy)methyl)benzamide;
N-(2-aminophenyl)-4-((1-methyl-1H-benzo[d]imidazol-2-ylthio)methyl)benzamide;
N-(2-aminophenyl)-4-((1-methyl-1H-benzo[d]imidazol-2-ylsulfinyl)methyl)benzamide;
N-(2-aminophenyl)-4-((1-methyl-1H-benzo[d]imidazol-2-ylsulfonyl)methyl)benzamide;
N-(2-aminophenyl)-3-fluoro-4-((1-methyl-1H-benzo[d]imidazol-2-ylamino)methyl)benzamide;
N-(2-aminophenyl)-3-fluoro-4-((1-methyl-1H-benzo[d]imidazol-2-yloxy)methyl)benzamide;
N-(2-aminophenyl)-3-fluoro-4-((1-methyl-1H-benzo[d]imidazol-2-ylthio)methyl)benzamide;
N-(2-aminophenyl)-3-fluoro-4-((1-methyl-1H-benzo[d]imidazol-2-ylsulfinyl)methyl)benzamide;
N-(2-aminophenyl)-3-fluoro-4-((1-methyl-1H-benzo[d]imidazol-2-ylsulfonyl)methyl)benzamide;
N-(2-aminophenyl)-4-((6-methoxy-1-methyl-1H-benzo[d]imidazol-2-ylamino)methyl)benzamide;
N-(2-aminophenyl)-4-((6-methoxy-1-methyl-1H-benzo[d]imidazol-2-yloxy)methyl)benzamide;
N-(2-aminophenyl)-4-((6-methoxy-1-methyl-1H-benzo[d]imidazol-2-ylthio)methyl)benzamide;
N-(2-aminophenyl)-4-((6-methoxy-1-methyl-1H-benzo[d]imidazol-2-ylsulfinyl)methyl)benzamide;
N-(2-aminophenyl)-4-((6-methoxy-1-methyl-1H-benzo[d]imidazol-2-ylsulfonyl)methyl)benzamide;
N-(2-amino-4-fluorophenyl)-4-((1-methyl-1H-benzo[d]imidazol-2-ylamino)methyl)benzamide;
N-(2-amino-4-fluorophenyl)-4-((1-methyl-1H-benzo[d]imidazol-2-yloxy)methyl)benzamide;
N-(2-amino-4-fluorophenyl)-4-((1-methyl-1H-benzo[d]imidazol-2-ylthio)methyl)benzamide;
N-(2-amino-4-fluorophenyl)-4-((1-methyl-1H-benzo[d]imidazol-2-ylsulfinyl)methyl)benzamide;
N-(2-amino-4-fluorophenyl)-4-((1-methyl-1H-benzo[d]imidazol-2-ylsulfonyl)methyl)benzamide;
3-acetamido-N-(2-aminophenyl)-4-((1-methyl-1H-benzo[d]imidazol-2-ylamino)methyl)benzamide;
3-acetamido-N-(2-aminophenyl)-4-((1-methyl-1H-benzo[d]imidazol-2-yloxy)methyl)benzamide;
3-acetamido-N-(2-aminophenyl)-4-((1-methyl-1H-benzo[d]imidazol-2-ylthio)methyl)benzamide;
3-acetamido-N-(2-aminophenyl)-4-((1-methyl-1H-benzo[d]imidazol-2-ylsulfinyl)methyl)benzamide;
3-acetamido-N-(2-aminophenyl)-4-((1-methyl-1H-benzo[d]imidazol-2-ylsulfonyl)methyl)benzamide;
N-(2-aminophenyl)-4-((1-(piperidin-3-yl)-1H-benzo[d]imidazol-2-ylamino)methyl)benzamide;
N-(2-aminophenyl)-4-((1-(piperidin-3-yl)-1H-benzo[d]imidazol-2-yloxy)methyl)benzamide;
N-(2-aminophenyl)-4-((1-(piperidin-3-yl)-1H-benzo[d]imidazol-2-ylthio)methyl)benzamide;
N-(2-aminophenyl)-4-((1-(piperidin-3-yl)-1H-benzo[d]imidazol-2-ylsulfinyl)methyl)benzamide;
N-(2-aminophenyl)-4-((1-(piperidin-3-yl)-1H-benzo[d]imidazol-2-ylsulfonyl)methyl)benzamide;
4-((1H-benzo[d]imidazol-2-ylsulfinyl)methyl)-N-(2-aminophenyl)benzamide;
4-((1H-benzo[d]imidazol-2-ylsulfonyl)methyl)-N-(2-aminophenyl)benzamide;
N-(2-aminophenyl)-4-((1-isopropyl-1H-benzo[d]imidazol-2-ylthio)methyl)benzamide;
N-(2-aminophenyl)-4-((1-cyclohexyl-1H-benzo[d]imidazol-2-ylthio)methyl)benzamide;
4-((3H-imidazo[4,5-b]pyridin-2-ylthio)methyl)-N-(2-aminophenyl)benzamide;
4-((3H-imidazo[4,5-c]pyridin-2-ylthio)methyl)-N-(2-aminophenyl)benzamide;
4-((3H-imidazo[4,5-c]pyridin-2-yloxy)methyl)-N-(2-aminophenyl)benzamide; and
5-((3H-imidazo[4,5-b]pyridin-2-yloxy)methyl)-N-(2-aminophenyl)thiophene-2-carboxamide.

It is noted that the compounds of the present invention may be in the form of a pharmaceutically acceptable salt, biohydrolyzable ester, biohydrolyzable amide, biohydrolyzable carbamate, solvate, hydrate or prodrug thereof. For example, the compound optionally comprises a substituent that is convertible in vivo to a different substituent such as a hydrogen.

It is further noted that the compounds of the present invention may optionally be solely or predominantly in the enol tautomer in its active state. In addition, the compounds of the present invention can be present in a mixture of stereoisomers. Alternatively, the compounds may comprise a single stereoisomer.

In another embodiment, the present invention relates to a pharmaceutical composition comprising a compound according to any one of the above embodiments and variations as an active ingredient. In one variation, the pharmaceutical composition is a solid formulation adapted for oral administration. In another variation, the pharmaceutical composition is a liquid formulation adapted for oral administration. In a further variation, the pharmaceutical composition is a tablet. In yet another variation, the pharmaceutical composition is a liquid formulation adapted for parenteral administration.

In a further embodiment, the present invention relates to a pharmaceutical composition comprising a compound according to any one of the above embodiments and variations wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, and intrathecally.

In yet another embodiment, the present invention relates to a kit comprising a compound according to any one of the above embodiments and variations and instructions. In one variation, the instructions comprise one or more forms of information selected from the group consisting of indicating a disease state for which the compound is to be administered, storage information for the compound, dosing information and instructions regarding how to administer the compound. In another variation, the kit comprises the compound in a multiple dose form.

In still another embodiment, the present invention relates to an article of manufacture comprising a compound according to any one of the above embodiments and variations and packaging materials. In one variation, the packaging material comprises a container for housing the compound. In one particular variation, the container comprises a label indicating one or more members of the group consisting of a disease state for which the compound is to be administered, storage information, dosing information and/or instructions regarding how to administer the composition. In another variation, the article of manufacture comprises the compound in a multiple dose form.

In another embodiment, the present invention relates to a therapeutic method comprising administering a compound according to any one of the above embodiments and variations to a subject.

In a further embodiment, the present invention relates to a method of inhibiting histone deacetylase comprising contacting histone deacetylase with a compound according to any one of the above embodiments and variations.

In still another embodiment, the present invention relates to a method of inhibiting histone deacetylase comprising causing a compound according to any one of the above embodiments and variations to be present in a subject in order to inhibit histone deacetylase in vivo.

In yet another embodiment, the present invention relates to a method of inhibiting histone deacetylase comprising administering a first compound to a subject that is converted in vivo to a second compound wherein the second compound inhibits histone deacetylase in vivo, the second compound being a compound according to any one of the above embodiments and variations.

In another embodiment, the present invention relates to a method of treating a disease state for which histone deacetylase possesses activity that contributes to the pathology and/or symptomology of the disease state, the method comprising causing a compound according to any one of the above embodiments and variations to be present in a subject in a therapeutically effective amount for the disease state.

In a further embodiment, the present invention relates to a method of treating a disease state for which histone deacetylase possesses activity that contributes to the pathology and/or symptomology of the disease state, the method comprising administering a first compound to a subject that is converted in vivo to a second compound according to any one of the above embodiments and variations, wherein the second compound is present in a subject in a therapeutically effective amount for the disease state.

In yet another embodiment, the present invention relates to a method of treating a disease state for which histone deacetylase possesses activity that contributes to the pathology and/or symptomology of the disease state, the method comprising administering a compound according to any one of the above embodiments and variations, wherein the compound is present in the subject in a therapeutically effective amount for the disease state.

In still another embodiment, the present invention relates to a method for treating cancer comprising administering a compound according to any one of the above embodiments and variations to a mammalian species in need thereof. In one variation, the cancer is selected from the group consisting of squamous cell carcinoma, astrocytoma, Kaposi's sarcoma, glioblastoma, non small-cell lung cancer, bladder cancer, head and neck cancer, melanoma, ovarian cancer, prostate cancer, breast cancer, small-cell lung cancer, glioma, colorectal cancer, genitourinary cancer and gastrointestinal cancer.

In another embodiment, the present invention relates to a method for treating inflammation, inflammatory bowel disease, psoriasis, or transplant rejection, comprising administering a compound according to any one of the above embodiments and variations to a mammalian species in need thereof.

In a further embodiment, the present invention relates to a method for treating arthritis comprising administering a compound according to any one of the above embodiments and variations to a mammalian species in need thereof.

In yet another embodiment, the present invention relates to a method for treating degenerative diseases of the eye comprising administering a compound according to any one of the above embodiments and variations to a mammalian species in need thereof.

In still another embodiment, the present invention relates to a method for treating multiple sclerosis, amyotrophic lateral sclerosis, thyroid neoplasm or Alzheimer's disease comprising administering a compound according to any one of the above embodiments and variations to a mammalian species in need thereof.

In each of the above embodiments and variations, the histone deacetylase is optionally a Class I histone deacetylase. In particular variations of each of the above embodiments and variations, the histone deacetylase is HDAC2 and/or HDAC8.

In another of its aspects, the invention is directed to methods for preparing the inhibitors of the invention. In one embodiment, the method comprises:
coupling a compound having the formula 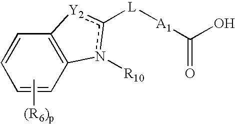
to a phenylenediamine compound having the formula 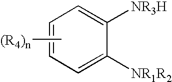
under conditions that form a compound having the formula 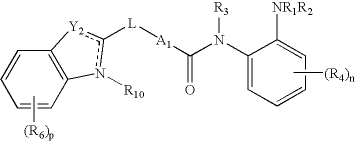
wherein
n is 0, 1, 2, 3, or 4;
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L is a linker comprising a backbone chain of 1 to 10 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁I₆)alkyl, and imino(C₁I₆)alkyl;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

In particular variations of the preceeding embodiment, (a) when Y₂ is NH; and R₁, R₂, and R₃ are all H; L is not —S—CH₂—; (b) when Y₂ is O; and R₁, R₂, and R₃ are all H; L is not —O—CH₂—; (c) when Y₂ is S; and R₁, R₂, and R₃ are all H; L is not —NH—CH₂—; (d) when Y₂ is S; and R₁, R₂, and R₃ are all H; L is not —NH—CH₂—CH═CH—; (e) when A₁ is 1,4-phenylene; L is methylene; R₁₀ is absent, Y₂ is NH, and p is 1; R₆ is not 5-F or 5-MeO; (f) when A₁ is 1,4-phenylene; L is methylene; R₁₀ is absent; Y₂ is NH; and p is 2; R₆ is not 5-Cl, 6-F or 5-F, 6-Cl.

In some embodiments, the method further comprises:
converting a carboxylic ester having the formula 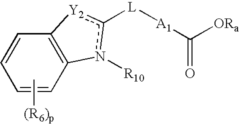
under conditions to yield a carboxylic acid having the formula 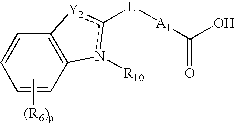
wherein
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L is a linker comprising a backbone chain of 1 to 10 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
F, is selected from the group consisting of (C₁₋₆)alkyl, (C₃₋₁₂)cycloalkyl, aryl and aryl(C₁₋₁₀)alkyl;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

In some embodiments, L is *NH—CH₂—L′, where
* indicates the point of attachment of L to the five membered ring containing Y₂, and
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₁₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In other embodiments, the method further comprises:
reacting a compound having the formula 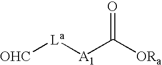
with a compound having the formula 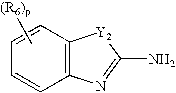
under conditions to form a compound having the formula 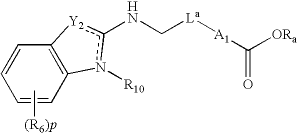
wherein
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₁₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl,
Y₂ is selected from the group consisting of CR₇R₈, NR₉, O, and S;
Rₐ is selected from the group consisting of (C₁₋₆)alkyl, (C₃₋₁₂)cycloalkyl, aryl and aryl(C₁₋₁₀)alkyl;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

In one variation of the above embodiments, L^{a} is a bond.

In some embodiments, L is *X—CH₂-L^{a}, where
* indicates the point of attachment of L to the five membered ring containing Y₂,
X is O or S; and
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₁₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

In another embodiment, the method further comprises:
reacting a compound having the formula 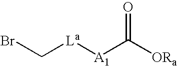
with a compound having the formula 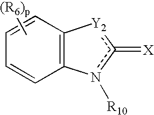
under conditions to form a compound having the formula 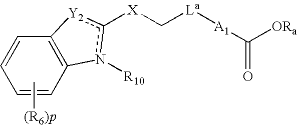
wherein
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₁₆)alkyl, (C₁₋₁₆)heteroalkyl, aryl, (C₁₋₁₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
X is 0 or S;
Y₂ is selected from the group consisting of CR₇R₈, NR₉, O, and S;
Rₐ is selected from the group consisting of (C₁₋₆)alkyl, (C₃₋₁₂)cycloalkyl, aryl and aryl(C₁₋₁₀)alkyl;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

In one variation of the above embodiments, X is O. In another variation of the above embodiments, X is S.

In another embodiment, when X is S, the method comprises:
oxidizing the sulfide of the compound having the formula 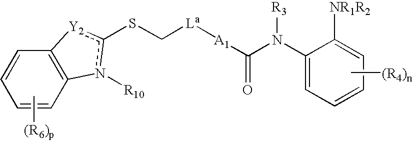
under conditions to yield the corresponding sulfoxide compound having the formula 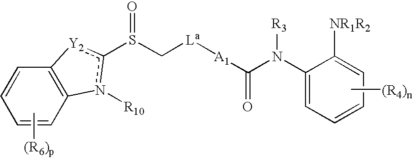
wherein
n is 0, 1, 2, 3, or 4;
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₁₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

In another embodiment, when X is S, the method comprises oxidizing the sulfide of the compound having the formula 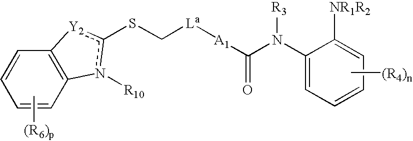
under conditions to yield the corresponding sulfone compound having the formula 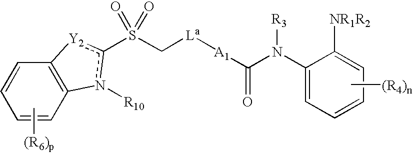
wherein
n is 0, 1, 2, 3, or 4;
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

In one variation of any one of the preceeding embodiments and variations, L^{a} is a bond.

In one variation of any one of the preceeding embodiments and variations, R₁ and R₂ is H. In another variation, R₁ and R₂ are both H.

In a further variation, R₃ is H.

In a still further variation of any one of the preceeding embodiments and variations, R₄ is hydrogen, halo, perhalo(C₁₋₃)alkyl, amino, cyano, nitro, thio, (C₁₋₅)alkyl, (C₃₋₅)cycloalkyl, hetero(C₃₋₅)cycloalkyl, aryl(C₁₋₃)alkyl, heteroaryl (C₁₋₃)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, alkenyl, alkynyl, carbonyl, and imino, each substituted or unsubstituted.

In a still further variation of any one of the preceeding embodiments and variations, A₁ is phenylene. In another still further variation, A₁ is 1,4-phenylene.

In a yet further variation of any one of the preceeding embodiments and variations, Y₂ is CR₇R₈ or NR₉ where R₉ is H. In another yet further variation Y₂ is CR₇R₈, or NR₉ where R₉ is not H.

In another of its aspects, the invention is directed to reagents which are useful in the preparation of the compounds of the invention. In one embodiment, the reagent is a compound having the formula 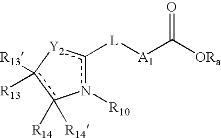
wherein
A₁ is selected from the group consisting of (C₃₋₁₂)cycloalkylene, hetero(C₃₋₁₂)cycloalkylene, arylene, and heteroarylene, each unsubstituted or substituted;
L is a linker comprising a backbone chain of 1 to 10 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
Rₐ is selected from the group consisting of (C₁₋₆)alkyl, (C₃₋₁₂)cycloalkyl, aryl and aryl(C₁₋₁₀)alkyl;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond; and
R₁₃, R₁₃′, R₁₄, and R₁₄′ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted, or R₁₃ and R₁₄ are taken together to form a ring, or R₁₃′ and R₁₄′ are absent when the atoms to which they are bound form part of a double bond.

In another embodiment, the reagent has the formula 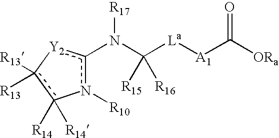
A₁ is selected from the group consisting of (C₃₋₁₂)cycloalkylene, hetero(C₃₋₁₂)cycloalkylene, arylene, and heteroarylene, each unsubstituted or substituted;
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
F, is selected from the group consisting of (C₁₋₆)alkyl, (C₃₋₁₂)cycloalkyl, aryl and aryl(C₁₋₁₀)alkyl;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond;
R₁₃, R₁₃′, R₁₄, and R₁₄′ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted, or R₁₃ and R₁₄ are taken together to form a ring, or R₁₃′ and R₁₄′ are absent when the atoms to which they are bound form part of a double bond;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted.

In one variation of the preceeding embodiment, R₁₇ is hydrogen. In another variation, at least one of R₁₅ and R₁₆ is hydrogen. In still another variation, both R₁₅ and R₁₆ are hydrogen. In yet another embodiment, R₁₅, R₁₆ and R₁₇ are all hydrogen.

In another embodiment, the reagent has the formula 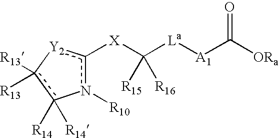
A₁ is selected from the group consisting of (C₃₋₁₂)cycloalkylene, hetero(C₃₋₁₂)cycloalkylene, arylene, and heteroarylene, each unsubstituted or substituted;
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₁₆)alkyl, (C₁₋₁₆)heteroalkyl, aryl, (C₁₋₁₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
X is 0 or S;
Y₂ is selected from the group consisting of CR₇R₈, NR₉, O, and S;
Rₐ is selected from the group consisting of (C₁₋₆)alkyl, (C₃₋₁₂)cycloalkyl, aryl and aryl(C₁₋₁₀)alkyl;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond;
R₁₃, R₁₃′, R₁₄, and R₁₄′ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted, or R₁₃ and R₁₄ are taken together to form a ring, or R₁₃′ and R₁₄′ are absent when the atoms to which they are bound form part of a double bond; and
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted.

In one variation of the preceeding embodiment, at least one of R₁₅ and R₁₆ is hydrogen. In still another variation, both R₁₅ and R₁₆ are hydrogen.

In another embodiment, the reagent has the formula 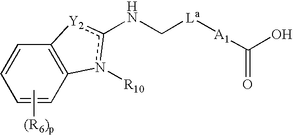
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

In another embodiment, the reagent has the formula 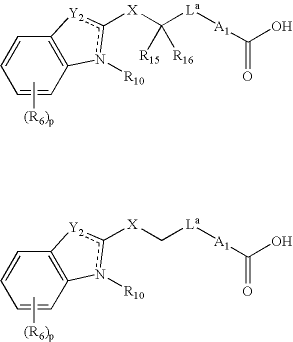
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₁₆)alkyl, (C₁₋₁₆)heteroalkyl, aryl, (C₁₋₁₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
X is 0 or S;
Y₂ is selected from the group consisting of CR₇R₈, NR₉, O, and S;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

In one variation of the preceeding embodiment, X is O. In another variation, X is S.

In another embodiment, the reagent has a formula 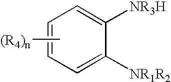
wherein
n is 0, 1, 2, 3, or 4;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted; and
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted.

In one variation of the preceeding embodiment, at least one of R₁ and R₂ is H. In another variation, R₁ and R₂ are both H. In a further variation, R₃ is H. In a still further variation, R₄ is hydrogen, halo, perhalo(C₁₋₃)alkyl, amino, cyano, nitro, thio, (C₁₋₅)alkyl, (C₃₋₅)cycloalkyl, hetero(C₃₋₅)cycloalkyl, aryl(C₁₋₃)alkyl, heteroaryl (C₁₋₃)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, alkenyl, alkynyl, carbonyl, and imino, each substituted or unsubstituted. In another variation, R₁, R₂, R₃, and R₄ are all hydrogen.

Salts, Hydrates, and Prodrugs of Histone Deacetylase Inhibitors

It should be recognized that the compounds of the present invention may be present and optionally administered in the form of salts, hydrates and prodrugs that are converted in vivo into the compounds of the present invention. For example, it is within the scope of the present invention to convert the compounds of the present invention into and use them in the form of their pharmaceutically acceptable salts derived from various organic and inorganic acids and bases in accordance with procedures well known in the art.

When the compounds of the present invention possess a free base form, the compounds can be prepared as a pharmaceutically acceptable acid addition salt by reacting the free base form of the compound with a pharmaceutically acceptable inorganic or organic acid, e.g., hydrohalides such as hydrochloride, hydrobromide, hydroiodide; other mineral acids and their corresponding salts such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the present invention include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate. It should be recognized that the free base forms will typically differ from their respective salt forms somewhat in physical properties such as solubility in polar solvents, but otherwise the salts are equivalent to their respective free base forms for the purposes of the present invention.

When the compounds of the present invention possess a free acid form, a pharmaceutically acceptable base addition salt can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g. potassium ethanolate and sodium propanolate; and various organic bases such as ammonium hydroxide, piperidine, diethanolamine and N-methylglutamine. Also included are the aluminum salts of the compounds of the present invention. Further base salts of the present invention include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine (benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine (tromethamine). It should be recognized that the free acid forms will typically differ from their respective salt forms somewhat in physical properties such as solubility in polar solvents, but otherwise the salts are equivalent to their respective free acid forms for the purposes of the present invention.

Compounds of the present invention that comprise basic nitrogen-containing groups may be quaternized with such agents as (C₁₋₄) alkyl halides, e.g., methyl, ethyl, iso-propyl and tert-butyl chlorides, bromides and iodides; di (C₁₋₄) alkyl sulfates, e.g., dimethyl, diethyl and diamyl sulfates; (C₁₀₋₁₈)alkyl halides, e.g., decyl, dodecyl, lauryl, myristyl and stearyl chlorides, bromides and iodides; and aryl (C₁₋₄) alkyl halides, e.g., benzyl chloride and phenethyl bromide. Such salts permit the preparation of both water-soluble and oil-soluble compounds of the present invention.

N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Prodrug derivatives of compounds according to the present invention can be prepared by modifying substituents of compounds of the present invention that are then converted in vivo to a different substituent. It is noted that in many instances, the prodrugs themselves also fall within the scope of the range of compounds according to the present invention. For example, prodrugs can be prepared by reacting a compound with a carbamylating agent (e.g., 1,1-acyloxyalkylcarbonochloridate, para-nitrophenyl carbonate, or the like) or an acylating agent. Further examples of methods of making prodrugs are described in Saulnier et al., 1994, Bioorganic and Medicinal Chemistry Letters, Vol. 4, p. 1985.

Protected derivatives of compounds of the present invention can also be made. Examples of techniques applicable to the creation of protecting groups and their removal can be found in T. W. Greene, Protecting Groups in Organic Synthesis, 3^{rd} edition, John Wiley & Sons, Inc. 1999.

Compounds of the present invention may also be conveniently prepared, or formed during the process of the invention, as solvates (e.g. hydrates). Hydrates of compounds of the present invention may be conveniently prepared by recrystallization from an aqueous/organic solvent mixture, using organic solvents such as dioxin, tetrahydrofuran or methanol.

A “pharmaceutically acceptable salt”, as used herein, is intended to encompass any compound according to the present invention that is utilized in the form of a salt thereof, especially where the salt confers on the compound improved pharmacokinetic properties as compared to the free form of compound or a different salt form of the compound. The pharmaceutically acceptable salt form may also initially confer desirable pharmacokinetic properties on the compound that it did not previously possess, and may even positively affect the pharmacodynamics of the compound with respect to its therapeutic activity in the body. An example of a pharmacokinetic property that may be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

Preparation of Histone Deacetylase Inhibitors

Various methods may be developed for synthesizing compounds according to the present invention. Representative methods for synthesizing these compounds are provided in the Examples. It is noted, however, that the compounds of the present invention may also be synthesized by other synthetic routes that others may devise.

It will be readily recognized that certain compounds according to the present invention have atoms with linkages to other atoms that confer a particular stereochemistry to the compound (e.g., chiral centers). It is recognized that synthesis of compounds according to the present invention may result in the creation of mixtures of different stereoisomers (enantiomers, diastereomers). Unless a particular stereochemistry is specified, recitation of a compound is intended to encompass all of the different possible stereoisomers.

Various methods for separating mixtures of different stereoisomers are known in the art. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) that they can be readily separated by taking advantage of these dissimilarities. For example, diastereomers can typically be separated by chromatography or by separation/resolution techniques based upon differences in solubility. A more detailed description of techniques that can be used to resolve stereoisomers of compounds from their racemic mixture can be found in Jean Jacques Andre Collet, Samuel H. Wilen, Enantiomers, Racemates and Resolutions, John Wiley & Sons, Inc. (1981).

Compositions Comprising Histone Deacetylase Inhibitors

A wide variety of compositions and administration methods may be used in conjunction with the HDAC inhibitors of the present invention. Such compositions may include, in addition to the HDAC inhibitors of the present invention, conventional pharmaceutical excipients, and other conventional, pharmaceutically inactive agents. Additionally, the compositions may include active agents in addition to the HDAC inhibitors of the present invention. These additional active agents may include additional compounds according to the invention, and/or one or more other pharmaceutically active agents.

The compositions may be in gaseous, liquid, semi-liquid or solid form, formulated in a manner suitable for the route of administration to be used. For oral administration, capsules and tablets are typically used. For parenteral administration, reconstitution of a lyophilized powder, prepared as described herein, is typically used.

Compositions comprising HDAC inhibitors of the present invention may be administered or coadministered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example by catheter or stent), subcutaneously, intraadiposally, intraarticularly, or intrathecally. The compounds and/or compositions according to the invention may also be administered or coadministered in slow release dosage forms.

The HDAC inhibitors and compositions comprising them may be administered or coadministered in any conventional dosage form. Co-administration in the context of this invention is intended to mean the administration of more than one therapeutic agent, one of which includes a HDAC inhibitor, in the course of a coordinated treatment to achieve an improved clinical outcome. Such co-administration may also be coextensive, that is, occurring during overlapping periods of time.

Solutions or suspensions used for parenteral, intradermal, subcutaneous, or topical application may optionally include one or more of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; agents for the adjustment of tonicity such as sodium chloride or dextrose, and agents for adjusting the acidity or alkalinity of the composition, such as alkaline or acidifying agents or buffers like carbonates, bicarbonates, phosphates, hydrochloric acid, and organic acids like acetic and citric acid. Parenteral preparations may optionally be enclosed in ampules, disposable syringes or single or multiple dose vials made of glass, plastic or other suitable material.

When HDAC inhibitors according to the present invention exhibit insufficient solubility, methods for solubilizing the compounds may be used. Such methods are known to those of skill in this art, and include, but are not limited to, using cosolvents, such as dimethylsulfoxide (DMSO), using surfactants, such as TWEEN, or dissolution in aqueous sodium bicarbonate. Derivatives of the compounds, such as prodrugs of the compounds may also be used in formulating effective pharmaceutical compositions.

Upon mixing or adding HDAC inhibitors according to the present invention to a composition, a solution, suspension, emulsion or the like may be formed. The form of the resulting composition will depend upon a number of factors, including the intended mode of administration, and the solubility of the compound in the selected carrier or vehicle. The effective concentration needed to ameliorate the disease being treated may be empirically determined.

Compositions according to the present invention are optionally provided for administration to humans and animals in unit dosage forms, such as tablets, capsules, pills, powders, dry powders for inhalers, granules, sterile parenteral solutions or suspensions, and oral solutions or suspensions, and oil-water emulsions containing suitable quantities of the compounds, particularly the pharmaceutically acceptable salts, preferably the sodium salts, thereof. The pharmaceutically therapeutically active compounds and derivatives thereof are typically formulated and administered in unit-dosage forms or multiple-dosage forms. Unit-dose forms, as used herein, refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses that are not segregated in packaging.

In addition to one or more HDAC inhibitors according to the present invention, the composition may comprise: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to form a solution or suspension. If desired, the pharmaceutical composition to be administered may also contain minor amounts of auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known in the art, or will be apparent, to those skilled in this art; for example, see Remington's Pharmaceutical Sciences, Mack Publishing Company, Easton, Pa., 15th Edition, 1975. The composition or formulation to be administered will, in any event, contain a sufficient quantity of a HDAC inhibitor of the present invention to reduce HDAC activity in vivo, thereby treating the disease state of the subject.

Dosage forms or compositions may optionally comprise one or more HDAC inhibitors according to the present invention in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of one or more HDAC inhibitors, optionally 0.1-95%, and optionally 1-95%.

Salts, preferably sodium salts, of the HDAC inhibitors may be prepared with carriers that protect the compound against rapid elimination from the body, such as time release formulations or coatings. The formulations may further include other active compounds to obtain desired combinations of properties.

Formulations for Oral Administration

Oral pharmaceutical dosage forms may be as a solid, gel or liquid. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art.

In certain embodiments, HDAC inhibitors according to the present invention are provided as solid dosage forms, preferably capsules or tablets. The tablets, pills, capsules, troches and the like may optionally contain one or more of the following ingredients, or compounds of a similar nature: a binder; a diluent; a disintegrating agent; a lubricant; a glidant; a sweetening agent; and a flavoring agent.

Examples of binders that may be used include, but are not limited to, microcrystalline cellulose, gum tragacanth, glucose solution, acacia mucilage, gelatin solution, sucrose and starch paste.

Examples of lubricants that may be used include, but are not limited to, talc, starch, magnesium or calcium stearate, lycopodium and stearic acid.

Examples of diluents that may be used include, but are not limited to, lactose, sucrose, starch, kaolin, salt, mannitol and dicalcium phosphate.

Examples of glidants that may be used include, but are not limited to, colloidal silicon dioxide.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of coloring agents that may be used include, but are not limited to, any of the approved certified water soluble FD and C dyes, mixtures thereof, and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

If oral administration is desired, the salt of the compound may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Compounds according to the present invention may also be administered as a component of an elixir, suspension, syrup, wafer, sprinkle, chewing gum or the like. A syrup may optionally comprise, in addition to the active compounds, sucrose as a sweetening agent and certain preservatives, dyes and colorings and flavors.

The HDAC inhibitors of the present invention may also be mixed with other active materials that do not impair the desired action, or with materials that supplement the desired action, such as antacids, H2 blockers, and diuretics. For example, if a compound is used for treating asthma or hypertension, it may be used with other bronchodilators and antihypertensive agents, respectively.

Examples of pharmaceutically acceptable carriers that may be included in tablets comprising HDAC inhibitors of the present invention include, but are not limited to binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets may be compressed tablets to which different layers of pharmaceutically acceptable substances are applied. Film-coated tablets may be compressed tablets that have been coated with polymers or other suitable coating. Multiple compressed tablets may be compressed tablets made by more than one compression cycle utilizing the pharmaceutically acceptable substances previously mentioned. Coloring agents may also be used in tablets. Flavoring and sweetening agents may be used in tablets, and are especially useful in the formation of chewable tablets and lozenges.

Examples of liquid oral dosage forms that may be used include, but are not limited to, aqueous solutions, emulsions, suspensions, solutions and/or suspensions reconstituted from non-effervescent granules and effervescent preparations reconstituted from effervescent granules.

Examples of aqueous solutions that may be used include, but are not limited to, elixirs and syrups. As used herein, elixirs refer to clear, sweetened, hydroalcoholic preparations. Examples of pharmaceutically acceptable carriers that may be used in elixirs include, but are not limited to solvents. Particular examples of solvents that may be used include glycerin, sorbitol, ethyl alcohol and syrup. As used herein, syrups refer to concentrated aqueous solutions of a sugar, for example, sucrose. Syrups may optionally further comprise a preservative.

Emulsions refer to two-phase systems in which one liquid is dispersed in the form of small globules throughout another liquid. Emulsions may optionally be oil-in-water or water-in-oil emulsions. Examples of pharmaceutically acceptable carriers that may be used in emulsions include, but are not limited to non-aqueous liquids, emulsifying agents and preservatives.

Examples of pharmaceutically acceptable substances that may be used in non-effervescent granules, to be reconstituted into a liquid oral dosage form, include diluents, sweeteners and wetting agents.

Examples of pharmaceutically acceptable substances that may be used in effervescent granules, to be reconstituted into a liquid oral dosage form, include organic acids and a source of carbon dioxide.

Coloring and flavoring agents may optionally be used in all of the above dosage forms.

Particular examples of preservatives that may be used include glycerin, methyl and propylparaben, benzoic add, sodium benzoate and alcohol.

Particular examples of non-aqueous liquids that may be used in emulsions include mineral oil and cottonseed oil.

Particular examples of emulsifying agents that may be used include gelatin, acacia, tragacanth, bentonite, and surfactants such as polyoxyethylene sorbitan monooleate.

Particular examples of suspending agents that may be used include sodium carboxymethylcellulose, pectin, tragacanth, Veegum and acacia. Diluents include lactose and sucrose. Sweetening agents include sucrose, syrups, glycerin and artificial sweetening agents such as sodium cyclamate and saccharin.

Particular examples of wetting agents that may be used include propylene glycol monostearate, sorbitan monooleate, diethylene glycol monolaurate and polyoxyethylene lauryl ether.

Particular examples of organic acids that may be used include citric and tartaric acid.

Sources of carbon dioxide that may be used in effervescent compositions include sodium bicarbonate and sodium carbonate. Coloring agents include any of the approved certified water soluble FD and C dyes, and mixtures thereof.

Particular examples of flavoring agents that may be used include natural flavors extracted from plants such fruits, and synthetic blends of compounds that produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g. water, to be easily measured for administration.

Alternatively, liquid or semi-solid oral formulations may be prepared by dissolving or dispersing the active compound or salt in vegetable oils, glycols, triglycerides, propylene glycol esters (e.g. propylene carbonate) and other such carriers, and encapsulating these solutions or suspensions in hard or soft gelatin capsule shells. Other useful formulations include those set forth in U.S. Pat. Nos. Re 28,819 and 4,358,603.

Injectables, Solutions, and Emulsions

The present invention is also directed to compositions designed to administer the HDAC inhibitors of the present invention by parenteral administration, generally characterized by injection, either subcutaneously, intramuscularly or intravenously. Injectables may be prepared in any conventional form, for example as liquid solutions or suspensions, solid forms suitable for solution or suspension in liquid prior to injection, or as emulsions.

Examples of excipients that may be used in conjunction with injectables according to the present invention include, but are not limited to water, saline, dextrose, glycerol or ethanol. The injectable compositions may also optionally comprise minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the formulations includes intravenous, subcutaneous and intramuscular administrations. Preparations for parenteral administration include sterile solutions ready for injection, sterile dry soluble products, such as the lyophilized powders described herein, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

When administered intravenously, examples of suitable carriers include, but are not limited to physiological saline or phosphate buffered saline (PBS), and solutions containing thickening and solubilizing agents, such as glucose, polyethylene glycol, and polypropylene glycol and mixtures thereof.

Examples of pharmaceutically acceptable carriers that may optionally be used in parenteral preparations include, but are not limited to aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles that may optionally be used include Sodium Chloride Injection, Ringers Injection, Isotonic Dextrose Injection, Sterile Water Injection, Dextrose and Lactated Ringers Injection.

Examples of nonaqueous parenteral vehicles that may optionally be used include fixed oils of vegetable origin, cottonseed oil, corn oil, sesame oil and peanut oil.

Antimicrobial agents in bacteriostatic or fungistatic concentrations may be added to parenteral preparations, particularly when the preparations are packaged in multiple-dose containers and thus designed to be stored and multiple aliquots to be removed. Examples of antimicrobial agents that may be used include phenols or cresols, mercurials, benzyl alcohol, chlorobutanol, methyl and propyl p-hydroxybenzoic acid esters, thimerosal, benzalkonium chloride and benzethonium chloride.

Examples of isotonic agents that may be used include sodium chloride and dextrose. Examples of buffers that may be used include phosphate and citrate. Examples of antioxidants that may be used include sodium bisulfate. Examples of local anesthetics that may be used include procaine hydrochloride. Examples of suspending and dispersing agents that may be used include sodium carboxymethylcellulose, hydroxypropyl methylcellulose and polyvinylpyrrolidone. Examples of emulsifying agents that may be used include Polysorbate 80 (TWEEN 80). A sequestering or chelating agent of metal ions include EDTA.

Pharmaceutical carriers may also optionally include ethyl alcohol, polyethylene glycol and propylene glycol for water miscible vehicles and sodium hydroxide, hydrochloric acid, citric acid or lactic acid for pH adjustment.

The concentration of a HDAC inhibitor in the parenteral formulation may be adjusted so that an injection administers a pharmaceutically effective amount sufficient to produce the desired pharmacological effect. The exact concentration of a HDAC inhibitor and/or dosage to be used will ultimately depend on the age, weight and condition of the patient or animal as is known in the art.

Unit-dose parenteral preparations may be packaged in an ampoule, a vial or a syringe with a needle. All preparations for parenteral administration should be sterile, as is know and practiced in the art.

Injectables may be designed for local and systemic administration. Typically a therapeutically effective dosage is formulated to contain a concentration of at least about 0.1% w/w up to about 90% w/w or more, preferably more than 1% w/w of the HDAC inhibitor to the treated tissue(s). The HDAC inhibitor may be administered at once, or may be divided into a number of smaller doses to be administered at intervals of time. It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also vary with the age of the individual treated. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

The HDAC inhibitor may optionally be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the disease state and may be empirically determined.

Lyophilized Powders

The HDAC inhibitors of the present invention may also be prepared as lyophilized powders, which can be reconstituted for administration as solutions, emulsions and other mixtures. The lyophilized powders may also be formulated as solids or gels.

Sterile, lyophilized powder may be prepared by dissolving the compound in a sodium phosphate buffer solution containing dextrose or other suitable excipient. Subsequent sterile filtration of the solution followed by lyophilization under standard conditions known to those of skill in the art provides the desired formulation. Briefly, the lyophilized powder may optionally be prepared by dissolving dextrose, sorbitol, fructose, corn syrup, xylitol, glycerin, glucose, sucrose or other suitable agent, about 1-20%, preferably about 5 to 15%, in a suitable buffer, such as citrate, sodium or potassium phosphate or other such buffer known to those of skill in the art at, typically, about neutral pH. Then, a HDAC inhibitor is added to the resulting mixture, preferably above room temperature, more preferably at about 30-35° C., and stirred until it dissolves. The resulting mixture is diluted by adding more buffer to a desired concentration. The resulting mixture is sterile filtered or treated to remove particulates and to insure sterility, and apportioned into vials for lyophilization. Each vial may contain a single dosage or multiple dosages of the HDAC inhibitor.

Topical Administration

The HDAC inhibitors of the present invention may also be administered as topical mixtures. Topical mixtures may be used for local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The HDAC inhibitors may be formulated as aerosols for topical application, such as by inhalation (see, U.S. Pat. Nos. 4,044,126, 4,414,209, and 4,364,923, which describe aerosols for delivery of a steroid useful for treatment inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The HDAC inhibitors may also be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the HDAC inhibitor alone or in combination with other pharmaceutically acceptable excipients can also be administered.

Formulations for Other Routes of Administrations

Depending upon the disease state being treated, other routes of administration, such as topical application, transdermal patches, and rectal administration, may also be used. For example, pharmaceutical dosage forms for rectal administration are rectal suppositories, capsules and tablets for systemic effect. Rectal suppositories are used herein mean solid bodies for insertion into the rectum that melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax, (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm. Tablets and capsules for rectal administration may be manufactured using the same pharmaceutically acceptable substance and by the same methods as for formulations for oral administration.

Examples of Formulations

The following are particular examples of oral, intravenous and tablet formulations that may optionally be used with compounds of the present invention. It is noted that these formulations may be varied depending on the particular compound being used and the indication for which the formulation is going to be used.

ORAL FORMULATION

| | | |
|---|---|---|
| Compound of the Present Invention | 10-100 | mg |
| Citric Acid Monohydrate | 105 | mg |
| Sodium Hydroxide | 18 | mg |
| Flavoring | | |
| Water | q.s. to 100 | mL |

INTRAVENOUS FORMULATION

| Compound of the Present Invention | 0.1-10 | mg |
|---|---|---|
| Dextrose Monohydrate | q.s. to make isotonic | |
| Citric Acid Monohydrate | 1.05 | mg |
| Sodium Hydroxide | 0.18 | mg |
| Water for Injection | q.s. to 1.0 | mL |

TABLET FORMULATION

| | |
|---|---|
| Compound of the Present Invention | 1% |
| Microcrystalline Cellulose | 73% |
| Stearic Acid | 25% |
| Colloidal Silica | 1%. |

Kits Comprising HDAC Inhibitors

The invention is also directed to kits and other articles of manufacture for treating diseases associated with HDACs. It is noted that diseases are intended to cover all conditions for which the HDACs possess activity that contributes to the pathology and/or symptomology of the condition.

In one embodiment, a kit is provided that comprises a composition comprising at least one HDAC inhibitor of the present invention in combination with instructions. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a composition comprising at least one HDAC inhibitor of the present invention in combination with packaging materials. The packaging material may comprise a container for housing the composition. The container may optionally comprise a label indicating the disease state for which the composition is to be administered, storage information, dosing information and/or instructions regarding how to administer the composition. The kit may also optionally comprise additional components, such as syringes for administration of the composition. The kit may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Combination Therapy

A wide variety therapeutic agents may have a therapeutic additive or synergistic effect with HDAC inhibitors according to the present invention. Such therapeutic agents may additively or synergistically combine with the HDAC inhibitors to inhibit undesirable cell growth, such as inappropriate cell growth resulting in undesirable benign conditions or tumor growth.

In one embodiment, a method is provided for treating a cell proliferative disease state comprising treating cells with a compound according to the present invention in combination with an anti-proliferative agent, wherein the cells are treated with the compound according to the present invention before, at the same time, and/or after the cells are treated with the anti-proliferative agent, referred to herein as combination therapy. It is noted that treatment of one agent before another is referred to herein as sequential therapy, even if the agents are also administered together. It is noted that combination therapy is intended to cover when agents are administered before or after each other (sequential therapy) as well as when the agents are administered at the same time.

Examples of therapeutic agents that may be used in combination with HDAC inhibitors include, but are not limited to, anticancer agents, alkylating agents, antibiotic agents, antimetabolic agents, hormonal agents, plant-derived agents, and biologic agents.

Alkylating agents are polyfunctional compounds that have the ability to substitute alkyl groups for hydrogen ions. Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). These compounds react with phosphate, amino, hydroxyl, sulfihydryl, carboxyl, and imidazole groups. Under physiological conditions, these drugs ionize and produce positively charged ion that attach to susceptible nucleic acids and proteins, leading to cell cycle arrest and/or cell death. Combination therapy including a HDAC inhibitor and an alkylating agent may have therapeutic synergistic effects on cancer and reduce sides affects associated with these chemotherapeutic agents.

Antibiotic agents are a group of drugs that produced in a manner similar to antibiotics as a modification of natural products. Examples of antibiotic agents include, but are not limited to, anthracyclines (e.g. doxorubicin, daunorubicin, epirubicin, idarubicin and anthracenedione), mitomycin C, bleomycin, dactinomycin, plicatomycin. These antibiotic agents interferes with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and forms an activated complex, which then binds to bases of DNA, causing strand scissions and cell death. Combination therapy including a HDAC inhibitor and an antibiotic agent may have therapeutic synergistic effects on cancer and reduce sides affects associated with these chemotherapeutic agents.

Antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. Combination therapy including a HDAC inhibitor and a antimetabolic agent may have therapeutic synergistic effects on cancer and reduce sides affects associated with these chemotherapeutic agents.

Hormonal agents are a group of drug that regulate the growth and development of their target organs. Most of the hormonal agents are sex steroids and their derivatives and analogs thereof, such as estrogens, androgens, and progestins. These hormonal agents may serve as antagonists of receptors for the sex steroids to down regulate receptor expression and transcription of vital genes. Examples of such hormonal agents are synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone. Combination therapy including a HDAC inhibitor and a hormonal agent may have therapeutic synergistic effects on cancer and reduce sides affects associated with these chemotherapeutic agents.

Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), taxanes (e.g., paclitaxel and docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission. Combination therapy including a HDAC inhibitor and a plant-derived agent may have therapeutic synergistic effects on cancer and reduce sides affects associated with these chemotherapeutic agents.

Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. Combination therapy including a HDAC inhibitor and a biologic agent may have therapeutic synergistic effects on cancer, enhance the patient's immune responses to tumorigenic signals, and reduce potential sides affects associated with this chemotherapeutic agent.

Cytokines possess profound immunomodulatory activity. Some cytokines such as interleukin-2 (IL-2, aldesleukin) and interferon have demonstrated antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself. Examples of interleukins that may be used in conjunction with HDAC inhibitor include, but are not limited to, interleukin 2 (IL-2), and interleukin 4 (IL-4), interleukin 12 (IL-12).

Interferon include more than 23 related subtypes with overlapping activities, all of the IFN subtypes within the scope of the present invention. IFN has demonstrated activity against many solid and hematologic malignancies, the later appearing to be particularly sensitive.

Other cytokines that may be used in conjunction with a HDAC inhibitor include those cytokines that exert profound effects on hematopoiesis and immune functions. Examples of such cytokines include, but are not limited to erythropoietin, granulocyte-CSF (filgrastin), and granulocyte, macrophage-CSF (sargramostim). These cytokines may be used in conjunction with a HDAC inhibitor to reduce chemotherapy-induced myelopoietic toxicity.

Other immuno-modulating agents other than cytokines may also be used in conjunction with a HDAC inhibitor to inhibit abnormal cell growth. Examples of such immuno-modulating agents include, but are not limited to bacillus Calmette-Guerin, levamisole, and octreotide, a long-acting octapeptide that mimics the effects of the naturally occurring hormone somatostatin.

Monoclonal antibodies against tumor antigens are antibodies elicited against antigens expressed by tumors, preferably tumor-specific antigens. For example, monoclonal antibody HERCEPTIN® (Trastruzumab) is raised against human epidermal growth factor receptor 2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein. Combination therapy including HDAC inhibitor and HERCEPTIN® may have therapeutic synergistic effects on tumors, especially on metastatic cancers.

Another example of monoclonal antibodies against tumor antigens is RITUXAN® (Rituximab) that is raised against CD20 on lymphoma cells and selectively deplete normal and malignant CD20⁺ pre-B and mature B cells. RITUXAN® is used as single agent for the treatment of patients with relapsed or refractory low-grade or follicular, CD20+, B cell non-Hodgkin's lymphoma. Combination therapy including HDAC inhibitor and RITUXAN® may have therapeutic synergistic effects not only on lymphoma, but also on other forms or types of malignant tumors.

Tumor suppressor genes are genes that function to inhibit the cell growth and division cycles, thus preventing the development of neoplasia. Mutations in tumor suppressor genes cause the cell to ignore one or more of the components of the network of inhibitory signals, overcoming the cell cycle check points and resulting in a higher rate of controlled cell growth—cancer. Examples of the tumor suppressor genes include, but are not limited to, DPC-4, NF-1, NF-2, RB, p53, WT1, BRCA1 and BRCA2.

DPC-4 is involved in pancreatic cancer and participates in a cytoplasmic pathway that inhibits cell division. NF-J codes for a protein that inhibits Ras, a cytoplasmic inhibitory protein. NF-1 is involved in neurofibroma and pheochromocytomas of the nervous system and myeloid leukemia. NF-2 encodes a nuclear protein that is involved in meningioma, schwanoma, and ependymoma of the nervous system. RB codes for the pRB protein, a nuclear protein that is a major inhibitor of cell cycle. RB is involved in retinoblastoma as well as bone, bladder, small cell lung and breast cancer. P53 codes for p53 protein that regulates cell division and can induce apoptosis. Mutation and/or inaction of p53 is found in a wide ranges of cancers. WT1 is involved in Wilms tumor of the kidneys. BRCA1 is involved in breast and ovarian cancer, and BRCA2 is involved in breast cancer. The tumor suppressor gene can be transferred into the tumor cells where it exerts its tumor suppressing functions. Combination therapy including a HDAC inhibitor and a tumor suppressor may have therapeutic synergistic effects on patients suffering from various forms of cancers.

Cancer vaccines are a group of agents that induce the body's specific immune response to tumors. Most of cancer vaccines under research and development and clinical trials are tumor-associated antigens (TAAs). TAA are structures (i.e. proteins, enzymes or carbohydrates) which are present on tumor cells and relatively absent or diminished on normal cells. By virtue of being fairly unique to the tumor cell, TAAs provide targets for the immune system to recognize and cause their destruction. Example of TAAs include, but are not limited to gangliosides (GM2), prostate specific antigen (PSA), alpha-fetoprotein (AFP), carcinoembryonic antigen (CEA) (produced by colon cancers and other adenocarcinomas, e.g. breast, lung, gastric, and pancreas cancer s), melanoma associated antigens (MART-1, gp 100, MAGE 1,3 tyrosinase), papillomavirus E6 and E7 fragments, whole cells or portions/lysates of antologous tumor cells and allogeneic tumor cells.

An adjuvant may be used to augment the immune response to TAAs. Examples of adjuvants include, but are not limited to, bacillus Calmette-Guerin (BCG), endotoxin lipopolysaccharides, keyhole limpet hemocyanin (GKLH), interleukin-2 (IL-2), granulocyte-macrophage colony-stimulating factor (GM-CSF) and cytoxan, a chemotherapeutic agent which is believe to reduce tumor-induced suppression when given in low doses.

### EXAMPLES

Preparation of Histone Deacetylase Inhibitors

Various methods may be developed for synthesizing compounds according to the present invention. Representative methods for synthesizing these compounds are provided in the Examples. It is noted, however, that the compounds of the present invention may also be synthesized by other synthetic routes that others may devise.

It will be readily recognized that certain compounds according to the present invention have atoms with linkages to other atoms that confer a particular stereochemistry to the compound (e.g., chiral centers). It is recognized that synthesis of compounds according to the present invention may result in the creation of mixtures of different stereoisomers (enantiomers, diastereomers). Unless a particular stereochemistry is specified, recitation of a compound is intended to encompass all of the different possible stereoisomers.

Various methods for separating mixtures of different stereoisomers are known in the art. For example, a racemic mixture of a compound may be reacted with an optically active resolving agent to form a pair of diastereoisomeric compounds. The diastereomers may then be separated in order to recover the optically pure enantiomers. Dissociable complexes may also be used to resolve enantiomers (e.g., crystalline diastereoisomeric salts). Diastereomers typically have sufficiently distinct physical properties (e.g., melting points, boiling points, solubilities, reactivity, etc.) that they can be readily separated by taking advantage of these dissimilarities. For example, diastereomers can typically be separated by chromatography or by separation/resolution techniques based upon differences in solubility. A more detailed description of techniques that can be used to resolve stereoisomers of compounds from their racemic mixture can be found in Jean Jacques Andre Collet, Samuel H. Wilen, Enantiomers, Racemates and Resolutions, John Wiley & Sons, Inc. (1981).

Compounds according to the present invention can also be prepared as a pharmaceutically acceptable acid addition salt by reacting the free base form of the compound with a pharmaceutically acceptable inorganic or organic acid. Alternatively, a pharmaceutically acceptable base addition salt of a compound can be prepared by reacting the free acid form of the compound with a pharmaceutically acceptable inorganic or organic base. Inorganic and organic acids and bases suitable for the preparation of the pharmaceutically acceptable salts of compounds are set forth in the definitions section of this Application. Alternatively, the salt forms of the compounds can be prepared using salts of the starting materials or intermediates.

The free acid or free base forms of the compounds can be prepared from the corresponding base addition salt or acid addition salt form. For example, a compound in an acid addition salt form can be converted to the corresponding free base by treating with a suitable base (e.g., ammonium hydroxide solution, sodium hydroxide, and the like). A compound in a base addition salt form can be converted to the corresponding free acid by treating with a suitable acid (e.g., hydrochloric acid, etc).

The N-oxides of compounds according to the present invention can be prepared by methods known to those of ordinary skill in the art. For example, N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compounds can be prepared from the N-oxide of an appropriate starting material.

Compounds in an unoxidized form can be prepared from N-oxides of compounds by treating with a reducing agent (e.g., sulfur, sulfur dioxide, triphenyl phosphine, lithium borohydride, sodium borohydride, phosphorus trichloride, tribromide, or the like) in an suitable inert organic solvent (e.g., acetonitrile, ethanol, aqueous dioxane, or the like) at 0 to 80° C.

Prodrug derivatives of the compounds can be prepared by methods known to those of ordinary skill in the art (e.g., for further details see Saulnier et al., 1994, Bioorganic and Medicinal Chemistry Letters, Vol. 4, p. 1985). For example, appropriate prodrugs can be prepared by reacting a non-derivatized compound with a suitable carbamylating agent (e.g., 1,1-acyloxyalkylcarbonochloridate, para-nitrophenyl carbonate, or the like).

Protected derivatives of the compounds can be made by methods known to those of ordinary skill in the art. A detailed description of the techniques applicable to the creation of protecting groups and their removal can be found in T. W. Greene, Protecting Groups in Organic Synthesis, 3^{rd} edition, John Wiley & Sons, Inc. 1999.

Compounds according to the present invention may be conveniently prepared, or formed during the process of the invention, as solvates (e.g. hydrates). Hydrates of compounds of the present invention may be conveniently prepared by recrystallization from an aqueous/organic solvent mixture, using organic solvents such as dioxin, tetrahydrofuran or methanol.

Compounds according to the present invention can also be prepared as their individual stereoisomers by reacting a racemic mixture of the compound with an optically active resolving agent to form a pair of diastereoisomeric compounds, separating the diastereomers and recovering the optically pure enantiomer. While resolution of enantiomers can be carried out using covalent diastereomeric derivatives of compounds, dissociable complexes are preferred (e.g., crystalline diastereoisomeric salts). Diastereomers have distinct physical properties (e.g., melting points, boiling points, solubilities, reactivity, etc.) and can be readily separated by taking advantage of these dissimilarities. The diastereomers can be separated by chromatography or, preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques Andre Collet, Samuel H. Wilen, Enantiomers, Racemates and Resolutions, John Wiley & Sons, Inc. (1981).

As used herein the symbols and conventions used in these processes, schemes and examples are consistent with those used in the contemporary scientific literature, for example, the Journal of the American Chemical Society or the Journal of Biological Chemistry. Standard single-letter or thee-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification:

| | |
|---|---|
| g (grams) | mg (milligrams) |
| L (liters) | mL (milliliters) |
| μL (microliters) | psi (pounds per square inch) |
| M (molar) | mM (millimolar) |
| i.v. (intravenous) | Hz (Hertz) |
| MHz (megahertz) | mol (moles) |
| mmol (millimoles) | RT (ambient temperature) |
| min (minutes); h (hours) | mp (melting point) |
| TLC (thin layer | Tr (retention time) |
| chromatography) | MeOH (methanol) |
| RP (reverse phase) | TEA (triethylamine) |
| i-PrOH (isopropanol) | TFAA (trifluoroacetic anhydride) |
| TFA (trifluoroacetic acid) | DMSO (dimethylsulfoxide) |
| THF (tetrahydrofuran) | DME (1,2-dimethoxyethane) |
| EtOAc (ethyl acetate) | DCE (dichloroethane) |
| DCM (dichloromethane) | DMPU (N,N′- |
| DMF (N,N-dimethylformamide) | dimethylpropyleneurea) |
| CDI (1,1-carbonyldiimidazole) | IBCF (isobutyl chloroformate) |
| HOAc or AcOH (acetic acid) | HOSu (N-hydroxysuccinimide) |
| HOBT (1-hydroxybenzotriazole) | Et₂O (diethyl ether) |
| EDCI (ethylcarbodiimide | BOC (tert-butyloxycarbonyl) |
| hydrochloride) | DCC (dicyclohexylcarbodiimide) |
| FMOC (9-fluorenylmethoxycarbonyl) | Ac (acetyl) |
| CBZ (benzyloxycarbonyl) | TMSE (2-(trimethylsilyl)ethyl) |
| atm (atmosphere) | TIPS (triisopropylsilyl) |
| TMS (trimethylsilyl) | DMAP (4-dimethylaminopyridine) |
| TBS (t-butyldimethylsilyl) | OMe (methoxy) |
| Me (methyl) | NaOAc (sodium acetate) |
| Et (ethyl) | SPA (Scintillation Proximity |
| tBu (tert-butyl) | Assay) |
| BOP (bis(2-oxo-3-oxazolidinyl) | TBAF (tetra-n-butylammonium |
| phosphinic chloride) | fluoride) |
| mCPBA (meta-chloroperbenzoic | ATP (Adenosine Triphophatase) |
| acid | EDTA (Ethylenediaminetetraacetic |
| BSA (Bovin Serum Albumin) | acid) |
| MOPS (Morpholinepropanesulfonic | HPLC (high pressure liquid |
| acid) | chromatography) |

All references to ether or Et₂O are to diethyl ether; brine refers to a saturated aqueous solution of NaCl. Unless otherwise indicated, all temperatures are expressed in ° C. (degrees Centigrade). All reactions conducted under an inert atmosphere at RT unless otherwise noted.

¹H NMR spectra were recorded on a Bruker Avance 400. Chemical shifts are expressed in parts per million (ppm). Coupling constants are in units of Hertz (Hz). Splitting patterns describe apparent multiplicities and are designated as s (singlet), d (doublet), t (triplet), q (quartet), m (multiplet), br (broad).

Low-resolution mass spectra (MS) and compound purity data were acquired on a Waters ZQ LC/MS single quadrupole system equipped with electrospray ionization (ESI) source, UV detector (220 and 254 nm), and evaporative light scattering detector (ELSD). Thin-layer chromatography was performed on 0.25 mm E. Merck silica gel plates (60F-254), visualized with UV light, 5% ethanolic phosphomolybdic acid, Ninhydrin or p-anisaldehyde solution. Flash column chromatography was performed on silica gel (230-400 mesh, Merck).

The starting materials and reagents used in preparing these compounds are either available from commercial suppliers such as the Aldrich Chemical Company (Milwaukee, Wis.), Bachem (Torrance, Calif.), Sigma (St. Louis, Mo.), or may be prepared by methods well known to a person of ordinary skill in the art, following procedures described in such standard references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The entire disclosure of each of the documents cited throughout this application are incorporated herein by reference.

Synthetic Schemes for Histone Deacetylase Inhibitors of the Present Invention

HDAC inhibitors according to the present invention may be synthesized according to the reaction schemes shown below. Other reaction schemes could be readily devised by those skilled in the art. It should also be appreciated that a variety of different solvents, temperatures and other reaction conditions can be varied to optimize the yields of the reactions.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in “Protective Groups in Organic Chemistry” John Wiley and Sons, 1991.

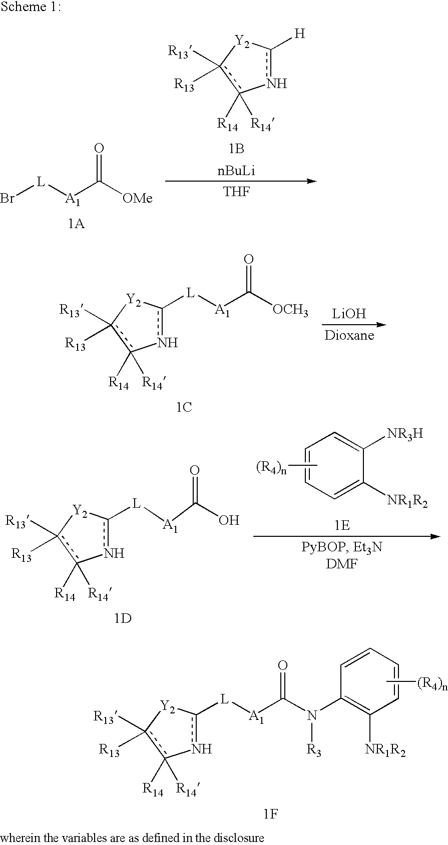

Compounds of the invention may be prepared by the route outlined in Scheme 1 above.

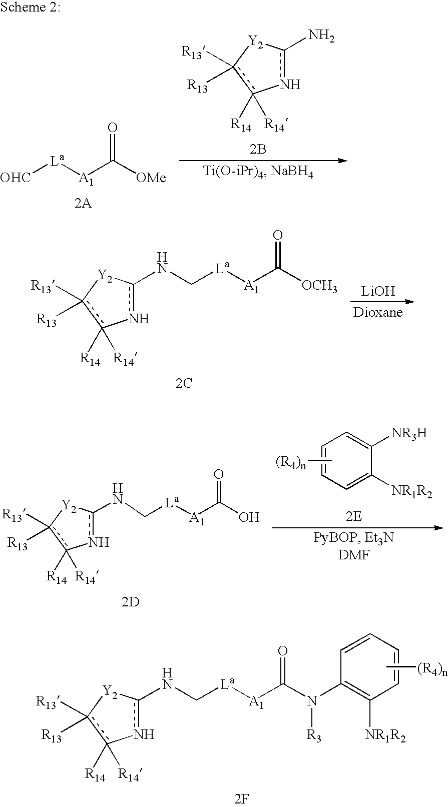

Specific compounds where L comprises a nitrogen atom bonds to the five-membered ring having Y₂ as a ring atom may be prepared according to the procedure outlined in Scheme 2.

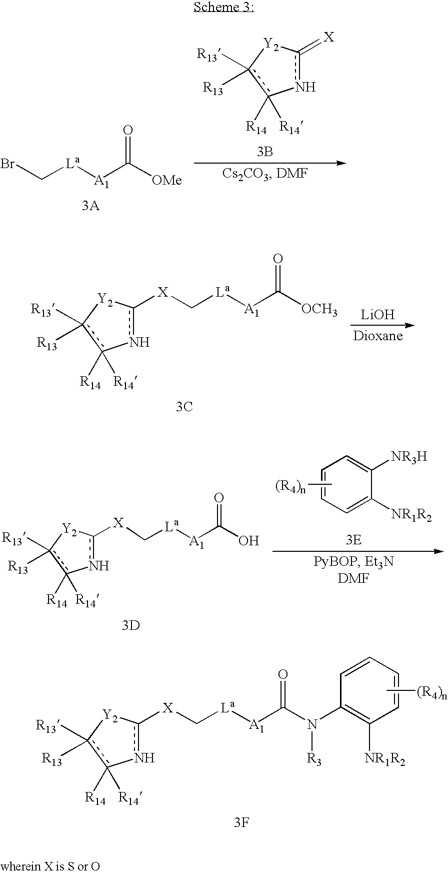

Specific compounds that L comprises a sulfur or oxygen atom bonds to the 5-membered ring having Y₂ as a ring atom may be prepared by the procedured outlined in Scheme 3.

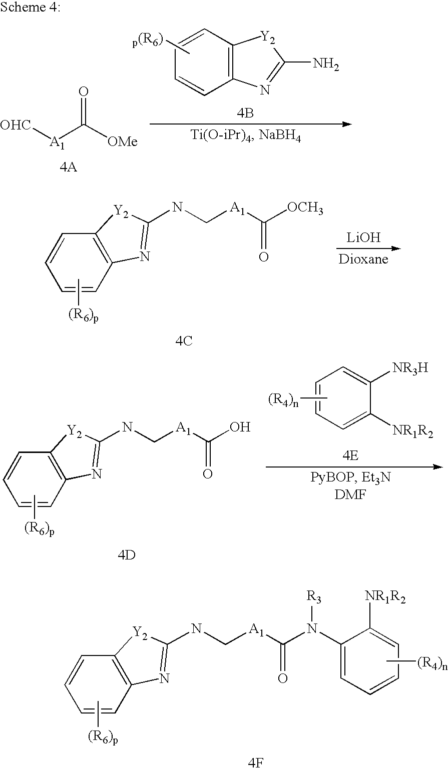

Reaction of Compound 4A with Compound 4B yields Compound 4C. Hydrolysis of Compound 4C yields carboxylic acid 4D. Reaction of Compound 4D with phenylenediamine 4E provides benzamide 4F.

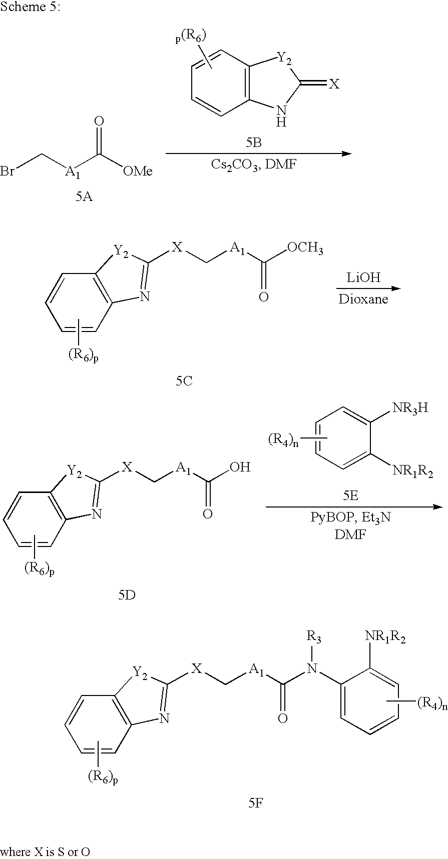

Reaction of Compound 5A with Compound 5B yields Compound 5C. Hydrolysis of Compound 5C yields carboxylic acid 5D. Reaction of Compound 5D with phenylenediamine 5E provides benzamide 5F.

For example, the above reaction schemes, and variations thereof, can be used to prepare the following:

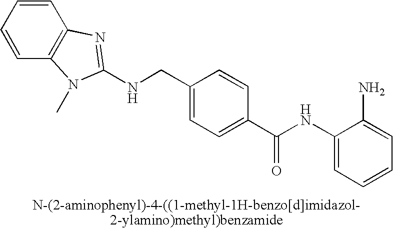

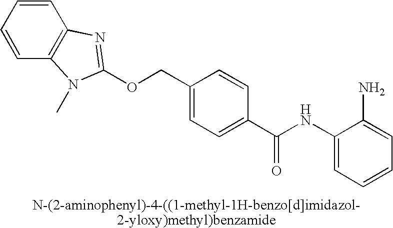

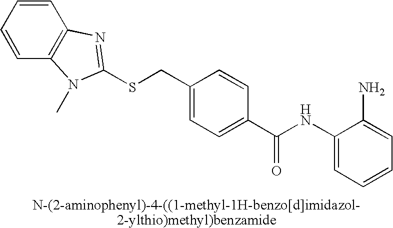

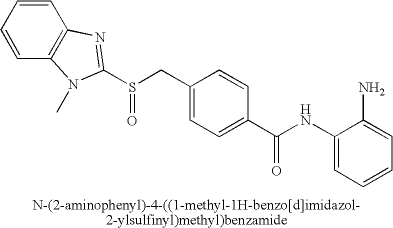

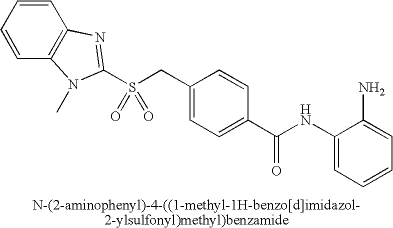

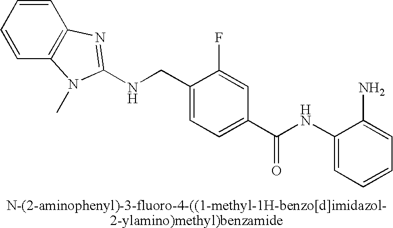

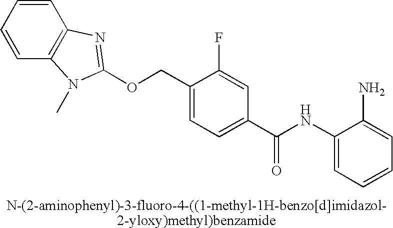

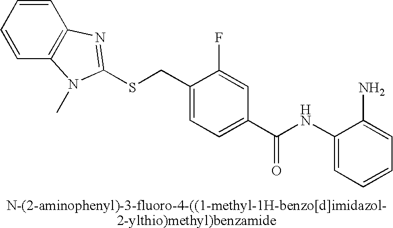

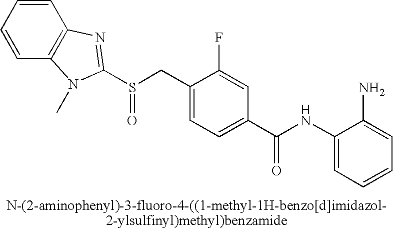

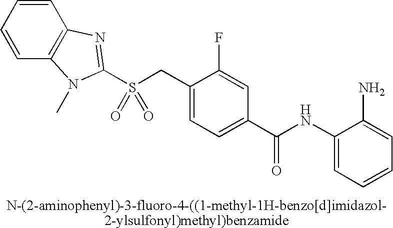

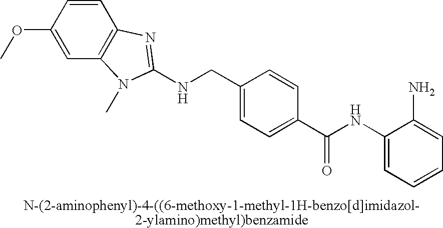

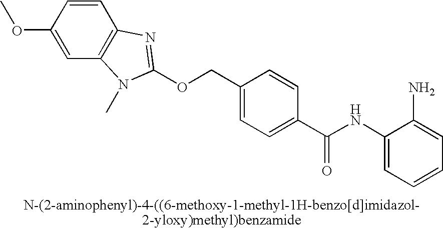

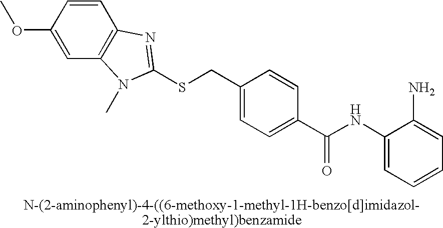

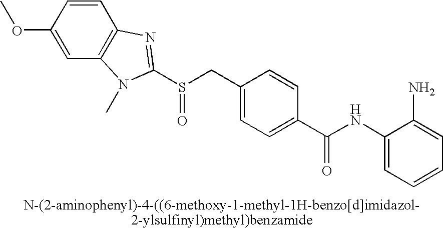

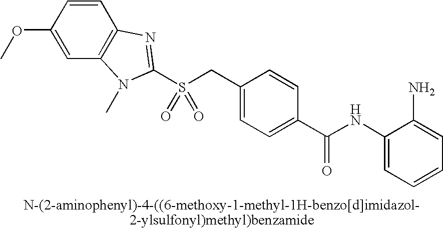

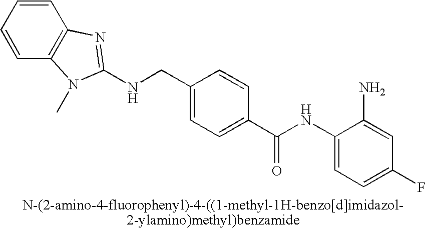

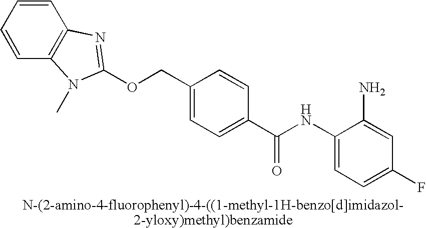

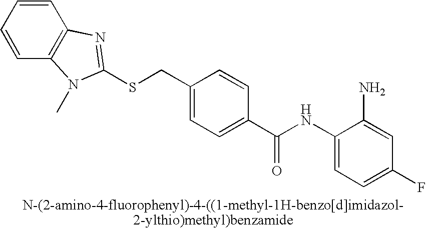

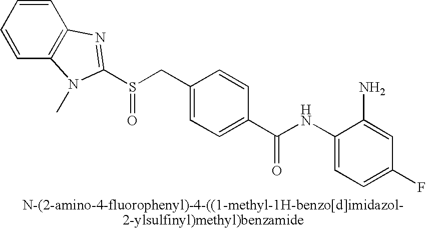

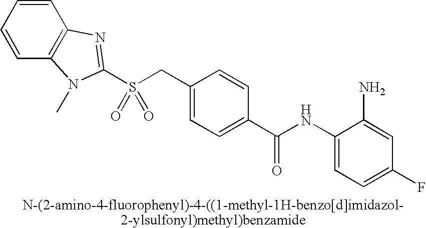

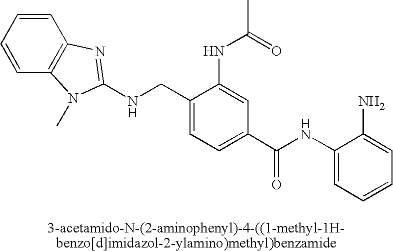

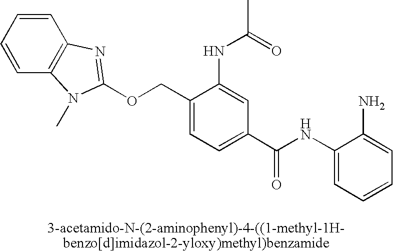

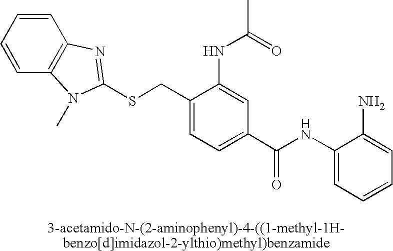

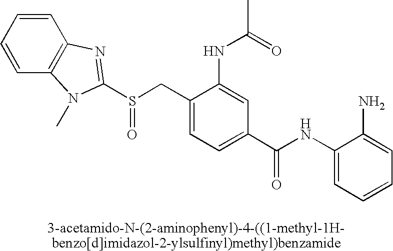

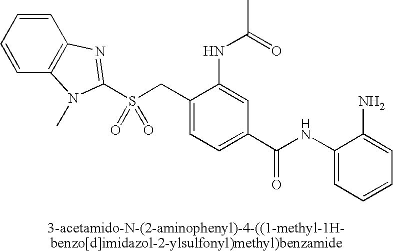

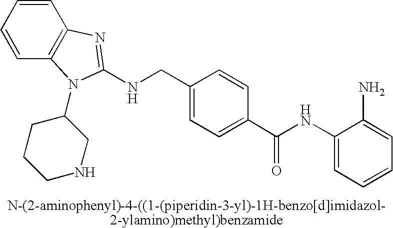

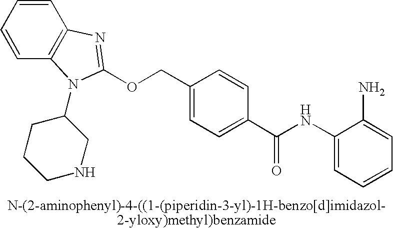

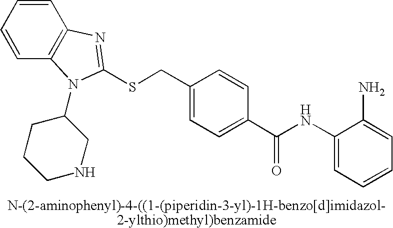

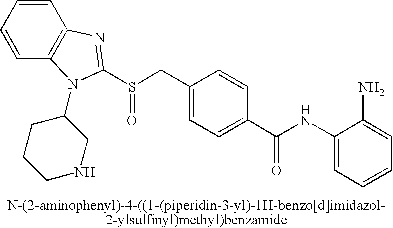

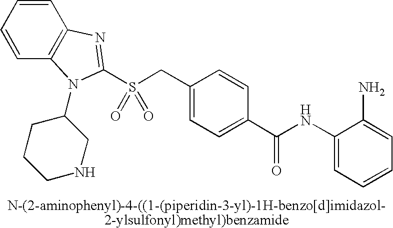

In each of the above reaction procedures or schemes, the various substituents may be selected from among the various substituents otherwise taught herein.

Descriptions of the syntheses of particular compounds according to the present invention based on the above reaction schemes are set forth herein.

Examples of Histone Deacetylase Inhibitors

The present invention is further exemplified, but not limited by, the following examples that describe the synthesis of particular compounds according to the invention.

### Example 1

### N-(2-aminophenyl)-4-((benzo[d]thiazol-2-ylamino)methyl)benzamide

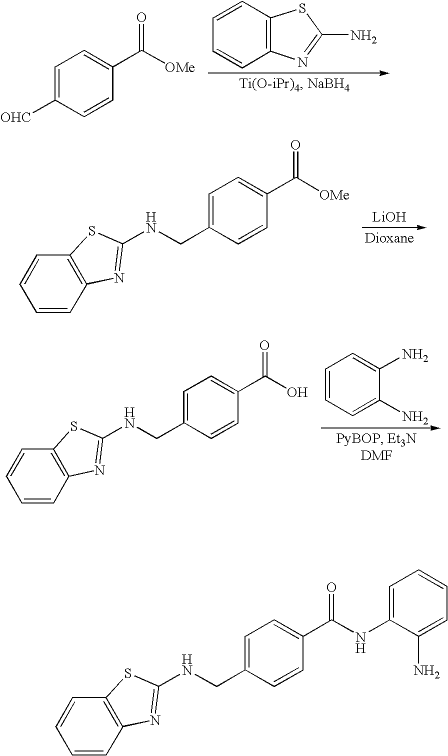

### Reductive Amination with Methyl 4-formylbenzoate

Methyl 4-formylbenzoate (3.4 mmol), benzo[d]thiazol-2-amine (0.86 mmol) and titanium isopropoxide (1.7 mmol) were suspended in diglyme (1 mL) and stirred at 60° C. for 30 min, then 23° C. for 30 min, and NaBH₄ (1.3 mmol) was added. The mixture was stirred at 23° C. for 3 hours, then at 60° C. overnight. The mixture was cooled to 0° C. and quenched with aqueous NH₄OH (2 M, 15 mL). The precipitate was filtered, washed with ether, and the aqueous layer was extracted with ether (3×25 mL). The organic layers were combined, dried (MgSO₄) and conc. in vacuo. The crude material was carried forward.

### Hydrolysis of 4-(Benzothiazol-2-ylaminomethyl)-benzoic acid methyl ester

A solution of 4-(Benzothiazol-2-ylaminomethyl)-benzoic acid methyl ester (0.25 mmol) in dioxane (1 mL) was treated with aqueous LiOH (1 M, 1.0 mL). After stirring at 23° C. for 2 h, the reaction was neutralized with aqueous HCl (1 M, 2.0 mL) and the resulting solid was isolated by filtration. The filter cake was rinsed with water and allowed to dry in vacuo to yield 4-(Benzothiazol-2-ylaminomethyl)-benzoic acid.

### Amideformation with 1,2-phenylenediamine

A solution of 4-(Benzothiazol-2-ylaminomethyl)-benzoic acid (0.1 mmol) in DMF (1 mL) was sequentially treated with PyBOP (0.12 mmol), 1,2-phenylenediamine (0.12 mmol) and Et₃N (0.3 mmol). After stirring at 23° C. for 4 h, the reaction was filtered and the crude material was purified by HPLC to yield N-(2-aminophenyl)-4-((benzo[d]thiazol-2-ylamino)methyl)benzamide. ¹H NMR (400 MHz, DMSO-D6) δ ppm 4.70 (d, 2H), 6.97-7.01 (m, 1H), 7.04-7.09 (m, 2H), 7.15-7.19 (m, 1H), 7.22-7.26 (m, 1H), 7.29 (d, 1H), 7.39 (d, 1H), 7.52 (d, 2H), 7.69-7.71 (d, 1H), 7.97-7.99 (d, 2H), 8.77 (s, 1H), 10.13 (s, 1H). ESI-MS: m/z 375.4 (M+H)⁺.

### Example 2

### 4-((1H-benzo[d]imidazol-2-ylamino)methyl)-N-(2-aminophenyl)benzamide

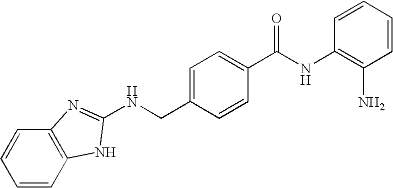

Example 2 was prepared analogously to Example 1. ¹H NMR (400 MHz, DMSO-D6) δ ppm 4.72 (d, 2H), 6.64 (m, 1H), 6.80 (d, 1H), 6.98 (m, 1H), 7.16 (d, 1H), 7.25-7.27 (m, 2H), 7.39-7.41 (m, 2H), 7.53 (d, 2H), 7.53-7.55 (m, 1H), 8.00 (d, 2H), 8.11 (d, 1H), 8.29 (d, 1H), 9.50 (s, 1H), 9.70 (s, 1H). ESI-MS: m/z 358.4 (M+H)⁺.

### Example 3

### 4-((1H-benzo[d]imidazol-2-ylthio)methyl)-N-(2-aminophenyl)benzamide

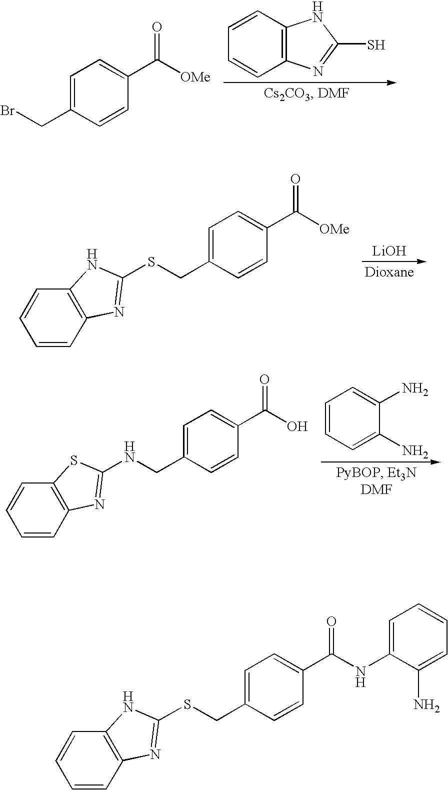

A solution of methyl 4-bromomethylbenzoate (0.5 mmol) in DMF (2.5 mL) was treated with 1H-benzo[d]imidazole-2-thiol (0.5 mmol) and solid Cs₂CO₃ (0.6 mmol). After stirring at 23° C. for 4 h, the reaction was poured into water (10 mL) and the resulting solid was isolated by filtration. The filter cake was rinsed with water and allowed to dry in vacuo to yield 4-(2-Oxo-2,3-dihydro-benzoimidazol-1-ylmethyl)-benzoic acid methyl ester.

Hydrolysis of the methyl ester and amide formation proceeded as in Example 1 to give 4-((1H-benzo[d]imidazol-2-ylthio)methyl)-N-(2-aminophenyl)benzamide. ¹H NMR (400 MHz, DMSO-D6) δ ppm 4.64 (s, 2H), 4.88 (s, 2H), 6.57 (d, J=7.07 Hz, 1H), 6.76 (d, J=7.83 Hz, 1H), 6.95 (d, J=7.07 Hz, 1H), 7.13 (d, J=5.43, Hz, 2H), 7.37 (s, 1H), 7.57 (d, J=8.08 Hz, 4H), 7.90 (d, J=7.83 Hz, 2H), 9.59 (s, 1H) 12.60 (s, 1H). ESI-MS: m/z 375.3 (M+H)⁺.

### Example 4

### 4-((3H-imidazo[4,5-b]pyridin-2-yloxy)methyl)-N-(2-aminophenyl)benzamide

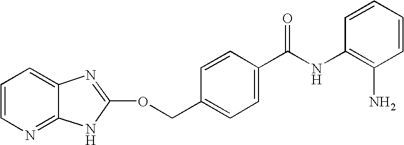

Example 4 was prepared analogously to Example 3. ¹H NMR (400 MHz, DMSO-D6) δ ppm 4.89 (s, 2H), 5.54 (s, 2H), 6.58 (s, 1H), 6.66-6.78 (m, 2H), 6.97 (dd, J=17.05, 7.45 Hz, 2H), 7.13 (s, 1H), 7.50 (d, J=8.08 Hz, 2H), 7.71 (d, J=6.57 Hz, 1H), 7.95 (d, J=7.58 Hz, 2H), 9.62 (s, 1H), 10.65 (s, 1H). ESI-MS: m/z 360.3 (M+H)⁺.

### Example 5

### 4-((1H-benzo[d]imidazol-2-ylsulfinyl)methyl)-N-(2-aminophenyl)benzamide

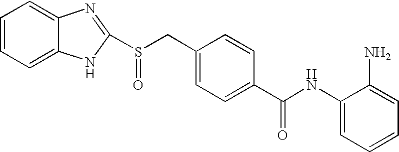

Example 5 was prepared analogously to Example 3. Oxidation of the sulfide to the sulfoxide was carried out using H₂O₂ (10 equivalents). ¹H NMR (400 MHz, DMSO-d₆) δ ppm 4.61 (d, J=12.88 Hz, 1H) 4.82 (d, J=12.88 Hz, 1H) 4.94 (br. s., 2H) 6.59 (td, J=7.58, 1.26 Hz, 1H) 6.77 (dd, J=8.08, 1.26 Hz, 1H) 6.97 (td, J=7.58, 1.52 Hz, 1H) 7.14 (d, J=7.07 Hz, 1H) 7.29 (d, J=8.08 Hz, 2H) 7.30-7.35 (m, 2H) 7.56 (br. s., 1H) 7.76 (br. s., 1H) 7.88 (d, J=8.08 Hz, 2H) 9.64 (s, 1H) 13.45 (br. s., 1H). ESI-MS: m/z 391.2 (M+H)⁺.

### Example 6

### 4-((1H-benzo[d]imidazol-2-ylsulfonyl)methyl)-N-(2-aminophenyl)benzamide

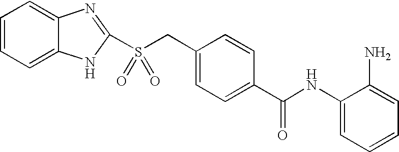

Example 6 was prepared analogously to Example 3. Oxidation of the sulfide to the sulfone was carried out using oxone (3 equivalents). ¹H NMR (400 MHz, DMSO-D6) δ ppm 4.90 (br. s., 2H) 5.12 (s, 2H) 6.58 (t, J=7.58 Hz, 1H) 6.77 (d, J=8.08 Hz, 1H) 6.96 (t, J=7.71 Hz, 1H) 7.14 (d, J=7.83 Hz, 1H) 7.37 (d, J=7.83 Hz, 2H) 7.41 (dd, J=5.68, 2.65 Hz, 2H) 7.61-7.85 (m, 2H) 7.90 (d, J=7.83 Hz, 2H) 9.65 (s, 1H) 13.90 (br. s., 1H). ESI-MS: m/z 407.3 (M+H)⁺.

### Example 7

### N-(2-aminophenyl)-4-((1-isopropyl-1H-benzo[d]imidazol-2-ylthio)methyl)benzamide

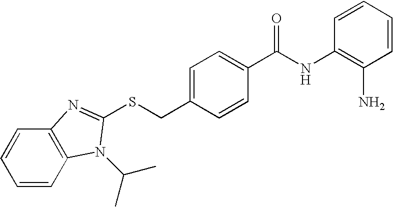

Example 7 was prepared analogously to Example 3. ¹H NMR (400 MHz, DMSO-D6) δ ppm 1.51 (d, J=6.82 Hz, 6H), 4.72 (br. s., 2H), 6.96 (m, 1H), 7.06 (m, 1H), 7.18 (dd, J=7.20, 1.14 Hz, 2H), 7.12-7.24 (m, 1H), 7.29 (m, 1H), 7.57-7.72 (m, 4H), 7.93 (d, J=8.08 Hz, 2H), 9.99 (br. s., 1H). ESI-MS: m/z 417.5 (M+H)⁺.

### Example 8

### N-(2-aminophenyl)-4-((1-cyclohexyl-1H-benzo[d]imidazol-2-ylthio)methyl)benzamide

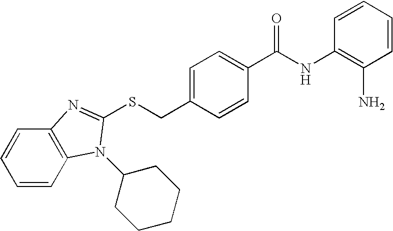

Example 8 was prepared analogously to Example 3. ¹H NMR (400 MHz, DMSO-D6) δ ppm 1.23 (br. s., 1H), 1.41 (br. s., 3H), 1.67 (br. s., 2H), 1.75 (s, 1H), 1.81 (br. s., 2H), 1.90 (br. s., 1H), 2.12 (br. s., 2H), 2.32 (br. s., 1H), 4.17 (br. s., 2H), 5.22 (br. s., 1 H), 5.68 (s, 1H), 6.71 (br. s., 1H), 6.87 (br. s., 1H), 7.03 (dd, J=6.57, 2.78 Hz, 1H), 7.11 (br. s., 1 H), 7.20 (d, J=2.78 Hz, 1H), 7.31 (br. s., 1H), 7.45 (br. s., 1H), 7.76 (br. s., 1H), 7.90 (br. s., 1H), 9.72 (br. s., 1H). ESI-MS: m/z 457.5 (M+H)⁺.

### Example 9

### 4-((3H-imidazo[4,5-b]pyridin-2-ylthio)methyl)-N-(2-aminophenyl)benzamide

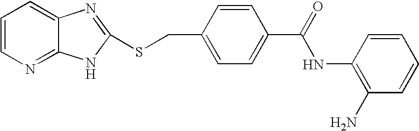

Example 9 was prepared analogously to Example 3. ¹H NMR (400 MHz, DMSO-D6) δ ppm. 4.70 (s, 3H) 6.80 (t, J=7.45 Hz, 1H) 6.93 (d, J=8.08 Hz, 1H) 7.07 (dd, J=15.16, 1.26 Hz, 1H) 7.20-7.24 (m, 2H) 7.63 (d, J=8.34 Hz, 2H) 7.93 (m, 3H) 8.26 (d, J=4.29 Hz, 1H) 9.83 (s, 1H). ESI-MS: m/z 376.3 (M+H)⁺.

### Example 10

### 4-((3H-imidazo[4,5-c]pyridin-2-ylthio)methyl)-N-(2-aminophenyl)benzamide

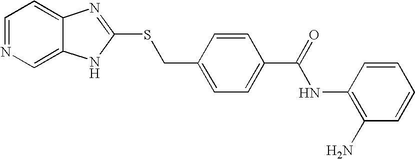

Example 10 was prepared analogously to Example 3. ¹H NMR (400 MHz, DMSO-D6) δ ppm 4.78 (s, 2H) 6.83 (t, J=7.45 Hz, 1H) 6.96 (d, J=7.83 Hz, 1H) 7.09 (t, J=7.58 Hz, 1H) 7.23 (d, J=7.83 Hz, 1H) 7.66 (d, J=8.08 Hz, 2H) 7.95 (d, J=8.08 Hz, 2H) 8.03 (d, J=6.32 Hz, 1H) 8.55 (d, J=6.57 Hz, 1H) 9.29 (s, 1H) 9.88 (s, 1H). ESI-MS: m/z 376.3 (M+H)⁺.

### Example 11

### 4-((3H-imidazo[4,5-c]pyridin-2-yloxy)methyl)-N-(2-aminophenyl)benzamide

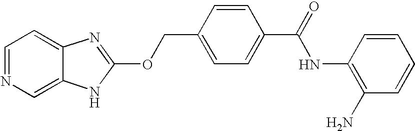

Example 11 was prepared analogously to Example 3. ¹H NMR (400 MHz, DMSO-D6) δ ppm 5.79 (s, 2H) 6.87 (t, J=7.33 Hz, 1H) 6.99 (d, J=7.83 Hz, 1H) 7.12 (t, J=8.34 Hz, 1H) 7.24 (d, J=7.83 Hz, 1H) 7.56-7.61 (m, 3H) 8.02 (d, J=8.08 Hz, 2H) 8.67 (dd, J=6.69, 1.39 Hz, 1H) 8.74 (d, J=0.76 Hz, 1H). ESI-MS: m/z 360.3 (M+H)⁺.

### Example 12

### 5-((3H-imidazo[4,5-b]pyridin-2-yloxy)methyl)-N-(2-aminophenyl)thiophene-2-carboxamide

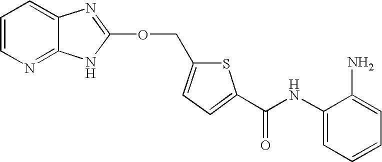

Example 12 was prepared analogously to Example 3. ¹H NMR (400 MHz, DMSO-D6) δ ppm 4.04 (br. s., 2H), 4.78 (br. s., 2H), 5.67 (br. s., 1H), 5.84 (dd, J=14.40, 7.58 Hz, 2H), 6.10 (d, J=6.82 Hz, 2H), 6.18 (d, J=7.07 Hz, 1H), 6.43 (br. s., 1H), 6.85 (d, J=6.57 Hz, 1H), 7.07 (br. s., 1H), 8.97 (br. s., 1H), 9.86 (br. s., 1H). ESI-MS: m/z 360.4 (M+H)⁺.

Biological Testing

The activity of compounds as HDAC inhibitors may be assayed in vitro, in vivo or in a cell line. Further, compounds according to the present invention may be screened for activity against one or more HDACs. Provided below are assays for activity against HDAC1, HDAC2, HDAC6 and HDAC8.

Purified HDAC1, HDAC2, HDAC6, and HDAC8 may be obtained as follows.

For HDAC1, DNA encoding residues 1-482 of the full-length sequence of the human enzyme may be amplified by PCR and cloned into the BamHI/XbaI sites of pFastbac (Invitrogen), which incorporates a Flag tag at both the N- and C-terminus. SEQ ID NO: 1 corresponds to residues 1-482 of HDAC1 with the N terminus and the N-and C-terminal Flag tag, plus an additional Met residue, which is a cloning artifact, inserted at the N-terminus. SEQ ID NO: 2 is the DNA sequence that was used to encode SEQ ID NO: 1.

For HDAC2, DNA encoding residues 1-488 of the full-length sequence of the human enzyme may be amplified by PCR and cloned into the BamnHI/SmaI sites of pFastbac (Invitrogen), which incorporates a 6-histidine tag at the C-terminus. SEQ ID NO: 3 corresponds to residues 1-488 with the C-terminal 6-histidine tag, plus four additional residues (Met-Gly-Ser inserted at the N-terminus and a Gly residue inserted in front of the 6-histidine tag) that are artifact of cloning. SEQ ID NO: 4 is the DNA sequence that was used to encode SEQ ID NO: 3.

For HDAC6, DNA encoding residues 73-845 of the human enzyme may be amplified by PCR and cloned into the SmaI site of pFastbac (Invitrogen), which incorporates a 6-histidine tag at the C-terminus. SEQ ID NO: 5 corresponds to residues 73-845 with the C-terminal 6-histidine tag, plus three additional residues (a Met-Pro residue pair inserted at the N-terminus and a Gly residue inserted before the 6×-histidine tag at the C-terminus) that are artifact of cloning. SEQ ID NO: 6 is the DNA sequence that was used to encode SEQ ID NO: 5.

For HDAC8, DNA encoding residues 1-377 corresponding to the entire sequence of the human enzyme may be amplified by PCR and cloned into the BamHI/SmaI sites of pFastbac (Invitrogen), which incorporates a 6-histidine tag at the N-terminus. SEQ ID NO: 7 corresponds to residues 1-377 with the N-terminal 6-histidine tag, plus two additional residues (a Met and a Pro inserted before and after the 6-histidine tag) that are artifact of cloning. SEQ ID NO: 8 is the DNA sequence that was used to encode SEQ ID NO: 7.

Recombinant baculovirus incorporating the HDAC constructs may be generated by transposition using the Bac-to-Bac system (Invitrogen). High-titer viral stocks may be generated by infection of Spodoptera frugiperda Sf9 cells; the expression of recombinant protein may be carried out by infection of Spodoptera frugiperda Sf9 or Trichoplusia ni Hi5 cells (Invitrogen) in 10L Wave Bioreactors (Wave Biotech).

Recombinant protein may be isolated from cellular extracts by passage over ProBond resin (Invitrogen), or Anti-Flag M2 Affinity Gel (Sigma) for HDAC1. Partially purified HDAC1 may then be further purified by high pressure liquid chromatography over a Mono Q column. Partially purified extracts of HDACs other than HDAC1 and HDAC6 may then be further purified by high pressure liquid chromatography over a BioSep S3000 gel filtration resin. The purity of HDAC proteins may be determined on denaturing SDS-PAGE gel. Purified HDACs may then be concentrated to a final concentration of 0.6 mg/mL for HDAC1, 10 mg/ml for HDAC2, 0.3 mg/mL for HDAC6, and 3 mg/mL for HDAC8. The proteins may be either stored at −78° C. in a buffer containing 25 mM TRIS-HCl pH 7.6, 150 mM NaCl, 0.1 mM EDTA and 0.25 mM TCEP or at −20° C. in the presence of glycerol (final concentration of glycerol at 50%). Alternatively, HDAC6 protein can be stored at −78° C. in a buffer containing 25 mM TRIS-HCl pH 7.2, 250 mM NaCl, and 5% glycerol.

The inhibitory properties of compounds relative to HDAC1, HDAC2, HDAC6 and HDAC8 may be determined using a white or black 384-well-plate format under the following reaction conditions: 25 mM Tris pH 8.0, 100 mM NaCl, 50 mM KCl, 0.1 mM EDTA, 0.01% Brij35, 0.1 nM TCEP. 50 μM tBoc-Lys(Ac)-AMC, 2% DMSO. Reaction product may be determined quantitatively by fluorescence intensity using a Fluorescence plate reader (Molecular Devices Gemini) with an excitation wavelength at 370 nM and emission at 480 nM (for white plates) or 465 nM (for black plates).

The assay reaction may be imitiated as follows: 5 μL of 150 μM tBoc-Lys(Ac)AMC was added to each well of the plate, followed by the addition of 5 μL of inhibitor (2 fold serial dilutions for 11 data points for each inhibitor) containing 6% DMSO. 5 μL of either HDAC1, HDAC2, HDAC6 or HDAC8 solution may be added to initiate the reaction (final enzyme concentrations were 2.5 nM for HDAC1, 1 nM for HDAC2, 2.5 nM for HDAC6 and 10 nM for HDAC8). The reaction mixture may then be incubated at room temperature for 60 min, and quenched and developed by addition of 5 μL of 10 mM phenanthroline and 4 mg/mL trypsin (final concentration of phenathroline is 2.5 mM, and trypsin is 1 mg/mL). Fluorescence intensities of the resulting reaction mixtures may be measured after a 30 minute incubation at room temperature.

IC₅₀ values may be calculated by non-linear curve fitting of the compound concentrations and fluorescence intensities to the standard IC₅₀ equation. As a reference point for this assay, suberanilohydroxamic acid (SAHA) showed an IC₅₀ of 63 nM for HDAC 1, 69 nM for HDAC2, 108 nM for HDAC6 and 242 nM for HDAC8.

IC₅₀ values for selected compounds of the present invention are given in Table 1.

TABLE 1
IC₅₀ of Exemplified Compounds Against HDAC2

| EXAMPLE | IC₅₀ (nM) |
|---|---|
| 1 | <100 |
| 2 | <100 |
| 3 | 100-1000 |
| 4 | 100-1000 |
| 5 | 100-1000 |
| 6 | 100-1000 |
| 7 | <100 |
| 8 | 100-1000 |
| 9 | <100 |
| 10 | <100 |
| 11 | 100-1000 |
| 12 | >1000 |

It will be apparent to those skilled in the art that various modifications and variations can be made in the compounds, compositions, kits, and methods of the present invention without departing from the spirit or scope of the invention. Thus, it is intended that the present invention cover the modifications and variations of this invention provided they come within the scope of the appended claims and their equivalents.

What is claimed is:

1. A compound comprising: 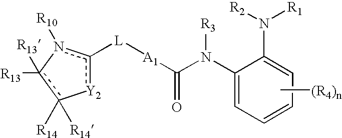
wherein:
n is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L is a linker comprising a backbone chain of 1 to 10 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond; and
R₁₃, R₁₃′, R₁₄, and R₁₄′ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted, or R₁₃ and R₁₄ are taken together to form a ring, or R₁₃′ and R₁₄′ are absent when the atoms to which they are bound form part of a double bond,
with the provisos that (a) Y₂ is not S when R₁, R₂, R₃, R₁O, R₁₃′, R₁₄, and R₁₄′ are all H; A₁ is phenylene; L is methylene; and R₁₃ is morpholino-4-ylmethyl; (b) L is not —S—CH₂— when R₁₃ and R₁₄ form a fused phenyl ring; Y₂ is NH; and R₁, R₂, and R₃ are all H; (c) L is not —O—CH₂— when R₁₃ and R₁₄ form a fused phenyl ring; Y₂ is O; and R₁, R₂, and R₃ are all H; (d) L is not —NH—CH₂— when R₁₃ and R₁₄ form a fused phenyl ring; Y₂ is S; and R₁, R₂, and R₃ are all H; (e) L is not —NH—CH₂—CH═CH— when R₁₃ and R₁₄ form a fused phenyl ring; Y₂ is S; and R₁, R₂, and R₃ are all H; and (f) when R₁₃ and R₁₃′ are taken together with the 5-membered ring to which they are attached to form benzimidazole; A₁ is 1,4-phenylene; and L is methylene; R₆ is not 5-F or 5-MeO when p is 1; and R₆ is not 5-Cl, 6-F or 5-F, 6-Cl when p is 2;
wherein
the compound includes any polymorph, solvate, ester, tautomer, enantiomer, and pharmaceutically acceptable salt of the compound.

2. The compound according to claim 1 comprising: 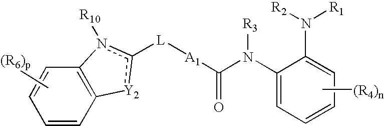
wherein:
p is 0, 1, 2, 3, or 4; and
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
with the provisos that (a) when Y₂ is NH; and R₁, R₂, and R₃ are all H; L is not —S—CH₂—; (b) when Y₂ is O; and R₁, R₂, and R₃ are all H; L is not —O—CH₂—; (c) when Y₂ is S; and R₁, R₂, and R₃ are all H; L is not —NH—CH₂—; (d) when Y₂ is S; and R₁, R₂, and R₃ are all H; L is not —NH—CH₂—CH═CH—; (e) when A₁ is 1,4-phenylene; L is methylene; R₁₀ is absent; Y₂ is NH; and p is 1; R₆ is not 5-F or 5-MeO; and (f) when A₁ is 1,4-phenylene;
L is methylene; R₁₀ is absent; Y₂ is NH; and p is 2; R₆ is not 5-Cl, 6-F or 5-F, 6-Cl.

3. The compound according to claim 1 comprising: 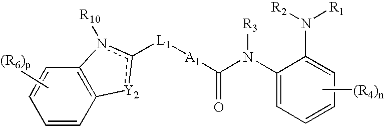
wherein:
p is 0, 1, 2, 3, or 4;
L₁ is selected from the group consisting of (C₁₋₆)alkylene, (C₂₋₆)alkenylene, and (C₂₋₆)alkynylene, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
with the proviso that
when A₁ is 1,4-phenylene; L₁ is methylene; R₁₀ is absent and Y₂ is NH; p is 1; R₆ is not 5-F or 5-MeO; and
when A₁ is 1,4-phenylene; L₁ is methylene; R₁₀ is absent and Y₂ is NH; p is 2; R₆ is not 5-Cl, 6-F or 5-F, 6-Cl.

4. The compound according to claim 1 comprising: 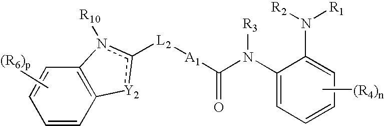
wherein:
p is 0, 1, 2, 3, or 4;
L₂ is selected from the group consisting of CR₁₅R₁₆, NR₁₇, S, S(O) and S(O)₂;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted,
with the proviso that
when A₁ is 1,4-phenylene; L₂ is methylene; R₁₀ is absent; Y₂ is NH; and p is 1; R₆ is not 5-F or 5-MeO; and
when A₁ is 1,4-phenylene; L₂ is methylene; R₁₀ is absent; Y₂ is NH; and p is 2; R₆ is not 5-Cl, 6-F or 5-F, 6-Cl.

5. The compound according to claim 1 comprising: 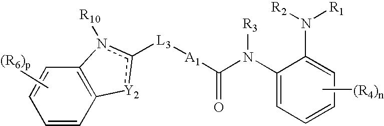
wherein:
p is 0, 1, 2, 3, or 4;
L₃ is selected from the group consisting of *CR₁₅R₁₆, *NR₁₇CR₁₅R₁₆, *OCR₁₅R₁₆, *SCR₁₅R₁₆, *S(O)CR₁₅R₁₆ and *S(O)₂CR₁₅R₁₆, where * indicates the point of attachment of L₃ to the five membered ring containing Y₂;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted,
with the proviso that
when A₁ is 1,4-phenylene; L₃ is methylene; R₁₀ is absent; Y₂ is NH; and p is 1; R₆ is not 5-F or 5-MeO; and
when A₁ is 1,4-phenylene; L₃ is methylene; R₁₀ is absent; Y₂ is NH; and p is 2; R₆ is not 5-Cl, 6-F or 5-F, 6-Cl.

6. The compound according to claim 1 comprising: 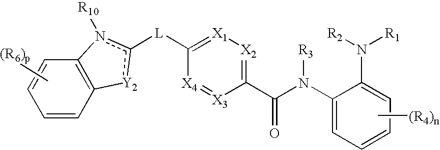
wherein:
p is 0, 1, 2, 3, or 4;
X₁, X₂, X₃, and X₄ are each independently selected from the group consisting of CR₅ and N;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
with the provisos that (a) when Y₂ is NH; X₁, X₂, X₃, and X₄ are all CH; and R₁, R₂, and R₃ are all H; L is not —S—CH₂—; (b) when Y₂ is O; X₁, X₂, X₃, and X₄ are all CH; and R₁, R₂, and R₃ are all H; L is not —O—CH₂—; (c) when Y₂ is S; X₁, X₂, X₃, and X₄ are all CH; and R₁, R₂, and R₃ are all H; L is not —NH—CH₂—; (d) when Y₂ is S; X₁, X₂, X₃, and X₄ are all CH; and R₁, R₂, and R₃ are all H; L is not —NH—CH₂—CH═CH—; (e) when X₁, X₂, X₃ and X₄ are all CH; L is methylene; R₁₀ is absent; Y₂ is NH; and p is 1; R₆ is not 5-F or 5-MeO; and (f) when X₁, X₂, X₃ and X₄ are all CH; L is methylene; R₁₀ is absent; Y₂ is NH; and p is 2; R₆ is not 5-Cl, 6-F or 5-F, 6-Cl.

7. The compound according to claim 1 comprising: 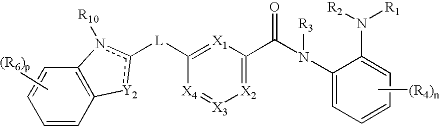
wherein:
p is 0, 1, 2, 3, or 4;
X₁, X₂, X₃, and X₄ are each independently selected from the group consisting of CR₅ and N;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
with the provisos that (a) when Y₂ is NH; X₁, X₂, X₃, and X₄ are all CH; and R₁, R₂, and R₃ are all H; L is not —S—CH₂—; (b) when Y₂ is O; X₁, X₂, X₃, and X₄ are all CH; and R₁, R₂, and R₃ are all H; L is not —O—CH₂—; (c) when Y₂ is S; X₁, X₂, X₃, and X₄ are all CH; and R₁, R₂, and R₃ are all H; L is not —NH—CH₂—; and/or (d) when Y₂ is S; X₁, X₂, X₃, and X₄ are all CH; and R₁, R₂, and R₃ are all H; L is not —NH—CH₂—CH═CH—.

8. The compound according to claim 1 comprising: 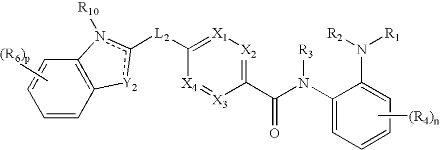
wherein:
p is 0, 1, 2, 3, or 4;
L₂ is selected from the group consisting of CR₁₅R₁₆, NR₁₇, S, S(O) and S(O)₂;
X₁, X₂, X₃, and X₄ are each independently selected from the group consisting of CR₅ and N;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₃)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted,
with the proviso that
when X₁, X₂, X₃ and X₄ are all CH; L₂ is methylene; R₁₀ is absent; Y₂ is NH; and p is 1; R₆ is not 5-F or 5-MeO, and
when X₁, X₂, X₃ and X₄ are all CH; L₂ is methylene; R₁₀ is absent; Y₂ is NH; and p is 2; R₆ is not 5-Cl, 6-F or 5-F, 6-Cl.

9. The compound according to claim 1 comprising: 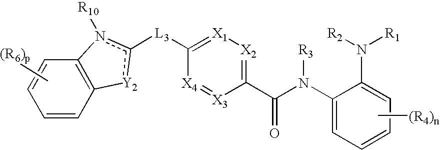
wherein:
p is 0, 1, 2, 3, or 4;
L₃ is selected from the group consisting of *CR₁₅R₁₆, *OCR₁₅R₁₆, *NR₁₇CR₁₅R₁₆, *SCR₁₅R₁₆, *S(O)CR₁₅R₁₆, and *S(O)₂CR₁₅R₁₆, where * indicates the point of attachment of L₃ to the ring containing Y₂;
X₁, X₂, X₃, and X₄ are each independently selected from the group consisting of CR₅ and N;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted;
with the proviso that
when X₁, X₂, X₃ and X₄ are all CH; L₃ is methylene; R₁₀ is absent; Y₂ is NH; and p is 1; R₆ is not 5-F or 5-MeO, and
when X₁, X₂, X₃ and X₄ are all CH; L₃ is methylene; R₁₀ is absent; Y₂ is NH; and p is 2; R₆ is not 5-Cl, 6-F or 5-F, 6-Cl.

10. The compound according to claim 1 comprising: 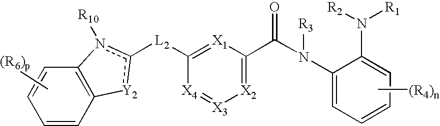
wherein:
p is 0, 1, 2, 3, or 4;
L₂ is selected from the group consisting of CR₁₅R₁₆, NR₁₇, S, S(O) and S(O)₂;
X₁, X₂, X₃, and X₄ are each independently selected from the group consisting of CR₅ and N;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted.

11. The compound according to claim 1 comprising: 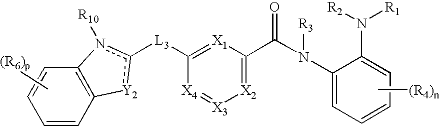
wherein:
p is 0, 1, 2, 3, or 4;
L₃ is selected from the group consisting of *CR₁₅R₁₆, *OCR₁₅R₁₆, *NR₁₇CR₁₅R₁₆, *SCR₁₅R₁₆, *S(O)CR₁₅R₁₆, and *S(O)₂CR₁₅R₁₆, where * indicates the point of attachment of L₃ to the ring containing Y₂;
X₁, X₂, X₃, and X₄ are each independently selected from the group consisting of CR₅ and N;
each R₅ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₁₅ and R₁₆ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted; and
R₁₇ is selected from the group consisting of hydrogen, nitro, thio, hydroxy, alkoxy, aryloxy, heteroaryloxy, carbonyl, amino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, (C₉₋₁₂)bicycloaryl, and hetero(C₄₋₁₂)bicycloaryl, each substituted or unsubstituted.

12. The compound according to claim 1, wherein L is selected from the group consisting of (C₁₋₆)alkylene, (C₂₋₆)alkenylene, and (C₂₋₆)alkynylene, each substituted or unsubstituted.

13. The compound according to claim 1, wherein L is a (C₁₋₄)alkylene, unsubstituted or substituted.

14. The compound according to claim 1, wherein L is a (C₁₋₃)alkylene, unsubstituted or substituted.

15. The compound according to claim 1, wherein L is a methylene, unsubstituted or substituted.

16. The compound according to claim 1, wherein at least one of R₁ and R₂ is H.

17. The compound according to claim 1, wherein R₁ and R₂ are both H.

18. The compound according to claim 1, wherein R₃ is H.

19. The compound according to claim 1, wherein R₄ is hydrogen, halo, perhalo(C₁₋₃)alkyl, amino, cyano, nitro, thio, (C₁₋₅)alkyl, (C₃₋₅)cycloalkyl, hetero(C₃₋₅)cycloalkyl, aryl(C₁₋₃)alkyl, heteroaryl (C₁₋₃)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, alkenyl, alkynyl, carbonyl, and imino, each substituted or unsubstituted.

20. The compound according to claim 5, wherein at least one of R₁₅ and R₁₆ is H.

21. The compound according to claim 5, wherein R₁₅ and R₁₆ are both H.

22. The compound according to claim 9, wherein at least one of R₁₅ and R₁₆ is H.

23. The compound according to claim 9, wherein R₁₅ and R₁₆ are both H.

24. The compound according to claim 1, wherein A₁ is phenylene.

25. The compound according to claim 1, wherein A₁ is 1,4-phenylene.

26. The compound according to claim 1, wherein Y₂ is CR₇R₈, or NR₉ where R₉ is not H.

27. The compound according to claim 1, wherein Y₂ is CR₇R₈, or NR₉ where R₉ is not H.

28. A compound selected from the group consisting of:
N-(2-aminophenyl)-4-((benzo[d]thiazol-2-ylamino)methyl)benzamide;
4-((1H-benzo[d]imidazol-2-ylamino)methyl)-N-(2-aminophenyl)benzamide;
4-((1H-benzo[d]imidazol-2-ylthio)methyl)-N-(2-aminophenyl)benzamide;
4-((3H-imidazo[4,5-b]pyridin-2-yloxy)methyl)-N-(2-aminophenyl)benzamide;
N-(2-aminophenyl)-4-((1-methyl-1H-benzo[d]imidazol-2-ylamino)methyl)benzamide;
N-(2-aminophenyl)-4-((1-methyl-1H-benzo[d]imidazol-2-yloxy)methyl)benzamide;
N-(2-aminophenyl)-4-((1-methyl-1H-benzo[d]imidazol-2-ylthio)methyl)benzamide;
N-(2-aminophenyl)-4-((1-methyl-1H-benzo[d]imidazol-2-ylsulfinyl)methyl)benzamide;
N-(2-aminophenyl)-4-((1-methyl-1H-benzo[d]imidazol-2-ylsulfonyl)methyl)benzamide;
N-(2-aminophenyl)-3-fluoro-4-((1-methyl-1H-benzo[d]imidazol-2-ylamino)methyl)benzamide;
N-(2-aminophenyl)-3-fluoro-4-((1-methyl-1H-benzo[d]imidazol-2-yloxy)methyl)benzamide;
N-(2-aminophenyl)-3-fluoro-4-((1-methyl-1H-benzo[d]imidazol-2-ylthio)methyl)benzamide;
N-(2-aminophenyl)-3-fluoro-4-((1-methyl-1H-benzo[d]imidazol-2-ylsulfinyl)methyl)benzamide;
N-(2-aminophenyl)-3-fluoro-4-((1-methyl-1H-benzo[d]imidazol-2-ylsulfonyl)methyl)benzamide;
N-(2-aminophenyl)-4-((6-methoxy-1-methyl-1H-benzo[d]imidazol-2-ylamino)methyl)benzamide;
N-(2-aminophenyl)-4-((6-methoxy-1-methyl-1H-benzo[d]imidazol-2-yloxy)methyl)benzamide;
N-(2-aminophenyl)-4-((6-methoxy-1-methyl-1H-benzo[d]imidazol-2-ylthio)methyl)benzamide;
N-(2-aminophenyl)-4-((6-methoxy-1-methyl-1H-benzo[d]imidazol-2-ylsulfinyl)methyl)benzamide;
N-(2-aminophenyl)-4-((6-methoxy-1-methyl-1H-benzo[d]imidazol-2-ylsulfonyl)methyl)benzamide;
N-(2-amino-4-fluorophenyl)-4-((1-methyl-1H-benzo[d]imidazol-2-ylamino)methyl)benzamide;
N-(2-amino-4-fluorophenyl)-4-((1-methyl-1H-benzo[d]imidazol-2-yloxy)methyl)benzamide;
N-(2-amino-4-fluorophenyl)-4-((1-methyl-1H-benzo[d]imidazol-2-ylthio)methyl)benzamide;
N-(2-amino-4-fluorophenyl)-4-((1-methyl-1H-benzo[d]imidazol-2-ylsulfinyl)methyl)benzamide;
N-(2-amino-4-fluorophenyl)-4-((1-methyl-1H-benzo[d]imidazol-2-ylsulfonyl)methyl)benzamide;
3-acetamido-N-(2-aminophenyl)-4-((1-methyl-1H-benzo[d]imidazol-2-ylamino)methyl)benzamide;
3-acetamido-N-(2-aminophenyl)-4-((1-methyl-1H-benzo[d]imidazol-2-yloxy)methyl)benzamide;
3-acetamido-N-(2-aminophenyl)-4-((1-methyl-1H-benzo[d]imidazol-2-ylthio)methyl)benzamide;
3-acetamido-N-(2-aminophenyl)-4-((1-methyl-1H-benzo[d]imidazol-2-ylsulfinyl)methyl)benzamide;
3-acetamido-N-(2-aminophenyl)-4-((1-methyl-1H-benzo[d]imidazol-2-ylsulfonyl)methyl)benzamide;
N-(2-aminophenyl)-4-((1-(piperidin-3-yl)-1H-benzo[d]imidazol-2-ylamino)methyl)benzamide;
N-(2-aminophenyl)-4-((1-(piperidin-3-yl)-1H-benzo[d]imidazol-2-yloxy)methyl)benzamide;
N-(2-aminophenyl)-4-((1-(piperidin-3-yl)-1H-benzo[d]imidazol-2-ylthio)methyl)benzamide;
N-(2-aminophenyl)-4-((1-(piperidin-3-yl)-1H-benzo[d]imidazol-2-ylsulfinyl)methyl)benzamide;
N-(2-aminophenyl)-4-((1-(piperidin-3-yl)-1H-benzo[d]imidazol-2-ylsulfonyl)methyl)benzamide;
4-((1H-benzo[d]imidazol-2-ylsulfinyl)methyl)-N-(2-aminophenyl)benzamide;
4-((1H-benzo[d]imidazol-2-ylsulfonyl)methyl)-N-(2-aminophenyl)benzamide;
N-(2-aminophenyl)-4-((1-isopropyl-1H-benzo[d]imidazol-2-ylthio)methyl)benzamide;
N-(2-aminophenyl)-4-((1-cyclohexyl-1H-benzo[d]imidazol-2-ylthio)methyl)benzamide;
4-((3H-imidazo[4,5-b]pyridin-2-ylthio)methyl)-N-(2-aminophenyl)benzamide;
4-((3H-imidazo[4,5-c]pyridin-2-ylthio)methyl)-N-(2-aminophenyl)benzamide;
4-((3H-imidazo[4,5-c]pyridin-2-yloxy)methyl)-N-(2-aminophenyl)benzamide; and
5-((3H-imidazo[4,5-b]pyridin-2-yloxy)methyl)-N-(2-aminophenyl)thiophene-2-carboxamide.

29. The compound according to claim 1, wherein the compound is in the form of a pharmaceutically acceptable salt.

30. The compound according to claim 1, wherein the compound is present in a mixture of stereoisomers.

31. The compound according to claim 1, wherein the compound comprises a single stereoisomer.

32. A pharmaceutical composition comprising as an active ingredient a compound according to claim 1.

33. The pharmaceutical composition according to claim 1, wherein the composition is a solid formulation adapted for oral administration.

34. The pharmaceutical composition according to claim 1, wherein the composition is a liquid formulation adapted for oral administration.

35. The pharmaceutical composition according to claim 1, wherein the composition is a tablet.

36. The pharmaceutical composition according to claim 1, wherein the composition is a liquid formulation adapted for parenteral administration.

37. The pharmaceutical composition comprising a compound according to claim 1, wherein the composition is adapted for administration by a route selected from the group consisting of orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, and intrathecally.

38. A kit comprising:
a compound according to claim 1; and
instructions which comprise one or more forms of information selected from the group consisting of indicating a disease state for which the compound is to be administered, storage information for the compound, dosing information and instructions regarding how to administer the compound.

39. The kit according to claim 38, wherein the kit comprises the compound in a multiple dose form.

40. An article of manufacture comprising:
a compound according to claim 33; and
packaging materials.

41. The article of manufacture according to claim 40, wherein the packaging material comprises a container for housing the compound.

42. The article of manufacture according to claim 41, wherein the container comprises a label indicating one or more members of the group consisting of a disease state for which the compound is to be administered, storage information, dosing information and/or instructions regarding how to administer the composition.

43. The article of manufacture according to claim 40, wherein the article of manufacture comprises the compound in a multiple dose form.

44. A therapeutic method comprising:
administering a compound according to claim 1 to a subject.

45. A method of inhibiting histone deacetylase comprising:
contacting histone deacetylase with a compound according to claim 1.

46. A method of inhibiting histone deacetylase comprising:
causing a compound according to claim 1 to be present in a subject in order to inhibit histone deacetylase in vivo.

47. A method of inhibiting histone deacetylase comprising:
administering a first compound to a subject that is converted in vivo to a second compound wherein the second compound inhibits histone deacetylase in vivo, the second compound being a compound according to claim 1.

48. A method of treating a disease state for which histone deacetylase possesses activity that contributes to the pathology and/or symptomology of the disease state, the method comprising:
causing a compound according to claim 1 to be present in a subject in a therapeutically effective amount for the disease state.

49. A method of treating a disease state for which histone deacetylase possesses activity that contributes to the pathology and/or symptomology of the disease state, the method comprising:
administering a first compound to a subject that is converted in vivo to a second compound according to claim 1, wherein the second compound is present in a subject in a therapeutically effective amount for the disease state.

50. A method of treating a disease state for which histone deacetylase possesses activity that contributes to the pathology and/or symptomology of the disease state, the method comprising:
administering a compound according to claim 1, wherein the compound is present in the subject in a therapeutically effective amount for the disease state.

51. A method for treating cancer comprising administering a composition according to claim 1 to a mammalian species in need thereof.

52. The method according to claim 50, wherein the cancer is selected from the group consisting of squamous cell carcinoma, astrocytoma, Kaposi's sarcoma, glioblastoma, non small-cell lung cancer, bladder cancer, head and neck cancer, melanoma, ovarian cancer, prostate cancer, breast cancer, small-cell lung cancer, glioma, colorectal cancer, genitourinary cancer and gastrointestinal cancer.

53. The method for treating inflammation, inflammatory bowel disease, psoriasis, or transplant rejection, comprising administering a compound according to claim 1 to a mammalian species in need thereof.

54. A method for treating arthritis comprising administering a compound according to claim 1 to a mammalian species in need thereof.

55. A method for treating degenerative diseases of the eye comprising administering a compound according to claim 1 to a mammalian species in need thereof.

56. A method for treating multiple sclerosis, amyotrophic lateral sclerosis, thyroid neoplasm or Alzheimer's disease comprising administering a compound according to claim 1 to a mammalian species in need thereof.

57. A method comprising:
coupling a compound having the formula 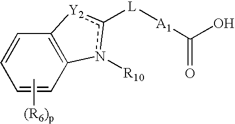
to a phenylenediamine compound having the formula 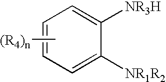
under conditions that form a compound having the formula 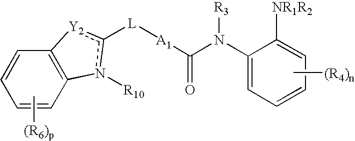
wherein
n is 0, 1, 2, 3, or 4;
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L is a linker comprising a backbone chain of 1 to 10 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, ary(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted;
R₇ and R₈ are each independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted, or R₈ is absent when the carbon to which it is bound forms part of a double bond;
R₉ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₉ is absent when the nitrogen to which it is bound forms part of a double bond; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond;
with the provisos that (a) when Y₂ is NH; and R₁, R₂, and R₃ are all H; L is not —S—CH₂—; (b) when Y₂ is O; and R₁, R₂, and R₃ are all H; L is not —O—CH₂—; (c) when Y₂ is S; and R₁, R₂, and R₃ are all H; L is not —NH—CH₂—; (d) when Y₂ is S; and R₁, R₂, and R₃ are all H; L is not —NH—CH₂—CH═CH—; (e) when A₁ is 1,4-phenylene; L is methylene; R₁₀ is absent; Y₂ is NH; and p is 1; R₆ is not 5-F or 5-MeO; and (f) when A₁ is 1,4-phenylene; L is methylene; R₁₀ is absent; Y₂ is NH; and p is 2; R₆ is not 5-Cl, 6-F or 5-F, 6-Cl.

58. The method according to claim 57 further comprising: converting a carboxylic ester having the formula 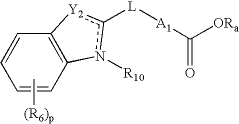
under conditions to yield a carboxylic acid having the formula 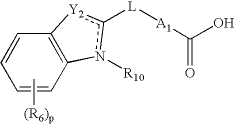
wherein
Rₐ is selected from the group consisting of (C₁₋₆)alkyl, (C₃₋₁₂)cycloalkyl, aryl and aryl(C₁₋₁₀)alkyl.

59. The method according to claim 58, wherein L is *NH—CH₂-L^{a}
where
* indicates the point of attachment of L to the five membered ring containing Y₂, and
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₁₆)alkyl, (C₁₋₁₆)heteroalkyl, aryl, (C₁₋₁₆)alkylaryl, aryl(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

60. The method according to claim 57 further comprising:
reacting a compound having the formula 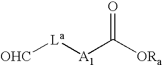
with a compound having the formula 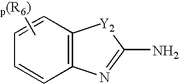
under conditions to form a compound having the formula

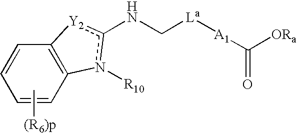

61. The method according to claim 60, wherein L^{a} is a bond.

62. The method according to claim 58, wherein L is *XCH₂L^{a},
where
* indicates the point of attachment of L to the five membered ring containing Y₂,
X is O or S, and
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, ary(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl.

63. The method according to claim 62 further comprising:
reacting a compound having the formula 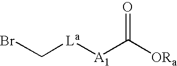
with a compound having the formula 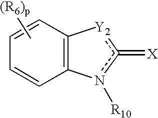
under conditions to form a compound having the formula

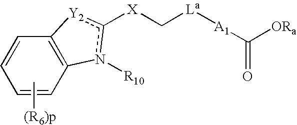

64. The method according to claim 63, wherein X is O.

65. The method according to claim 63, wherein X is S.

66. The method according to claim 65 further comprising
oxidizing the sulfide of the compound having the formula 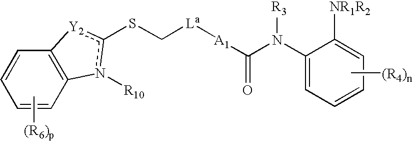
under conditions to yield the corresponding sulfoxide compound having the formula

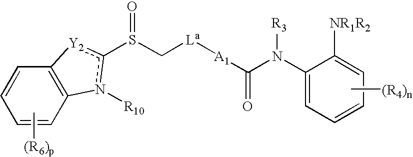

67. The method according to claim 66, wherein L^{a} is a bond.

68. The method according to claim 65 further comprising
oxidizing the sulfide of the compound having the formula 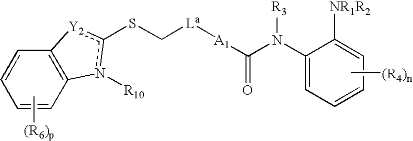
under conditions to yield the corresponding sulfone compound having the formula

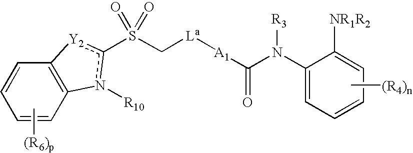

69. The method according to claim 68, wherein L^{a} is a bond.

70. The method according to claim 58, wherein Rₐ is methyl.

71. The method according to claim 60, wherein Rₐ is methyl.

72. The method according to claim 57, wherein at least one of R₁ and R₂ is H.

73. The method according to claim 57, wherein R₁ and R₂ are both H.

74. The method according to claim 57, wherein R₃ is H.

75. The method of according to claim 57, wherein R₄ is hydrogen, halo, perhalo(C₁₋₃)alkyl, amino, cyano, nitro, thio, (C₁₋₅)alkyl, (C₃₋₅)cycloalkyl, hetero(C₃₋₅)cycloalkyl, aryl(C₁₋₃)alkyl, heteroaryl (C₁₋₃)alkyl, carbonyl(C₁₋₃)alkyl, thiocarbonyl(C₁₋₃)alkyl, sulfonyl(C₁₋₃)alkyl, sulfinyl(C₁₋₃)alkyl, imino(C₁₋₃)alkyl, aryl, heteroaryl, hydroxy, alkoxy, alkenyl, alkynyl, carbonyl, and imino, each substituted or unsubstituted.

76. The method according to claim 57, wherein R₄ is hydrogen.

77. The method according to claim 57, wherein A₁ is phenylene.

78. The method according to claim 57, wherein A₁ is 1,4-phenylene.

79. The method according to claim 57, wherein Y₂ is CR₇R₈, or NR₉ where R₉ is not H.

80. The method according to claim 57, wherein Y₂ is CR₇R₈, or NR₉ where R₉ is not H.

81. A compound having the formula 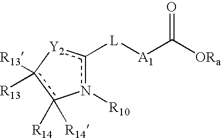
wherein
A₁ is selected from the group consisting of (C₃₋₁₂)cycloalkylene, hetero(C₃₋₁₂)cycloalkylene, arylene, and heteroarylene, each unsubstituted or substituted;
L is a linker comprising a backbone chain of 1 to 10 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, ary(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
R^{a} is selected from the group consisting of (C₁₋₆)alkyl, (C₃₋₁₂)cycloalkyl, aryl and aryl(C₁₋₁₀)alkyl;
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond; and
R₁₃, R₁₃′, R₁₄, and R₁₄′ are each independently selected from the group consisting of hydrogen, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl(C₁₋₁₀)alkyl, heteroaryl(C₁₋₅)alkyl, perhalo(C₁₋₁₀)alkyl, (C₃₋₁₂)cycloalkyl(C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino (C₁₋₁₀)alkyl, imino(C₁₋₃)alkyl, aryl, heteroaryl, (C₉₋₁₂)bicycloaryl, and hetero(C₄₋₁₂)bicycloaryl, each substituted or unsubstituted, or R₁₃ and R₁₄ are taken together to form a ring, or R₁₃′ and R₁₄′ are absent when the atoms to which they are bound form part of a double bond.

82. A compound having the formula 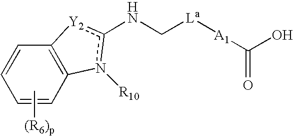
wherein
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, ary(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

83. A compound having the formula 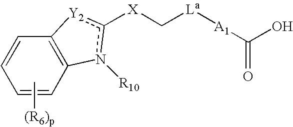
wherein
p is 0, 1, 2, 3, or 4;
A₁ is selected from the group consisting of (C₃₋₁₂)cycloalkylene, hetero(C₃₋₁₂)cycloalkylene, arylene, and heteroarylene, each unsubstituted or substituted;
L^{a} is a bond or is a linker comprising a backbone chain of 1 to 8 atoms comprising C, N, O, or S and may be optionally substituted with a substituent selected from the group consisting of halo, halo(C₁₋₆)alkyl, amino, nitro, cyano, thio, (C₁₋₆)alkyl, (C₃₋₇)cycloalkyl, (C₁₋₆)alkyl(C₃₋₇)cycloalkyl, (C₃₋₇)cycloalkyl(C₁₋₆)alkyl, hetero(C₃₋₇)cycloalkyl, (C₁₋₁₆)alkylhetero(C₃₋₇)cycloalkyl, hetero(C₃₋₇)cycloalkyl(C₁₋₆)alkyl, (C₁₋₆)heteroalkyl, aryl, (C₁₋₆)alkylaryl, ary(C₁₋₆)alkyl, heteroaryl, (C₁₋₆)alkylheteroaryl, heteroaryl(C₁₋₆)alkyl, carbonyl(C₁₋₆)alkyl, thiocarbonyl(C₁₋₅)alkyl, sulfonyl(C₁₋₆)alkyl, sulfinyl(C₁₋₆)alkyl, and imino(C₁₋₆)alkyl;
Y₂ is selected from the group consisting of CR₇R₈, NR₉, O, and S;
each R₆ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted; and
R₁₀ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted, or R₁₀ is absent when the nitrogen to which it is bound forms part of a double bond.

84. The compound according to claim 83, wherein X is O.

85. The compound according to claim 83, wherein X is S.

86. A compound having the formula 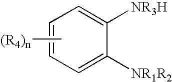
wherein
n is 0, 1, 2, 3, or 4;
R₁ and R₂ are each individually selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted;
R₃ is selected from the group consisting of hydrogen, (C₁₋₁₀)alkyl, (C₃₋₁₂)cycloalkyl, (C₃₋₁₂)cycloalkyl(C₁₋₅)alkyl, hetero(C₃₋₁₂)cycloalkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl, (C₉₋₁₂)bicycloaryl(C₁₋₅)alkyl, hetero(C₄₋₁₂)bicycloaryl, hetero(C₈₋₁₂)bicycloaryl(C₁₋₅)alkyl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, amino, aryl, heteroaryl, hydroxy, alkoxy, aryloxy, heteroaryloxy, carbonyl, imino, sulfonyl, and sulfinyl, each substituted or unsubstituted; and
each R₄ is independently selected from the group consisting of hydrogen, halo, perhalo(C₁₋₁₀)alkyl, amino, cyano, nitro, thio, (C₁₋₁₀)alkyl, (C₃₋₁₂)cycloalkyl, hetero(C₃₋₁₂)cycloalkyl, aryl(C₁₋₁₀)alkyl, heteroaryl (C₁₋₅)alkyl, (C₉₋₁₂)bicycloaryl, hetero(C₈₋₁₂)bicycloaryl, carbonyl (C₁₋₃)alkyl, thiocarbonyl (C₁₋₃)alkyl, sulfonyl (C₁₋₃)alkyl, sulfinyl (C₁₋₃)alkyl, imino (C₁₋₃)alkyl, aryl, heteroaryl, hydroxy, alkoxy, aryloxy, heteroaryloxy, alkenyl, alkynyl, carbonyl, imino, sulfonyl, and sulfinyl, each substituted or unsubstituted.

87. The compound of claim 86, wherein R₁, R₂, R₃, and R₄ are all hydrogen.
